(19) United States
(12) Patent Application Publication
(10) Pub. No.: US 2026/0250232 A1
(43) Pub. Date: Aug. 27, 2026

(54) FLUORINATED ESTERS, METHODS OF SYNTHESIZING THE SAME, AND USES OF THE SAME

(71) Applicant: Solstice Advanced Materials US, Inc., Morris Plains, NJ (US)

(72) Inventors: Xanath Ispizua Rodriguez, East Amherst, NY (US); David Charles Bock, Orchard Park, NY (US); Robert Joseph Stewart, East Aurora, NY (US); Sergey Nicholas Maximoff, Glenview, NY (US); Pramod Warrier, Buffalo, NY (US)

(21) Appl. No.: 19/549,288

(22) Filed: Feb. 25, 2026

Related U.S. Application Data
(60) Provisional application No. 63/762,925, filed on Feb. 25, 2025.
(60) Provisional application No. 63/843,733, filed on Jul. 14, 2025.
(60) Provisional application No. 63/899,624, filed on Oct. 15, 2025.
(60) Provisional application No. 63/988,883, filed on Feb. 23, 2026.

(51) Int. Cl. C07C 67/307; C07C 69/63; H01M 10/0525; H01M 10/054; H01M 10/0569

(52) U.S. Cl. CPC C07C 67/307; C07C 69/63; H01M 10/0525; H01M 10/054; H01M 10/0569; H01M 2300/0034; H01M 2300/0037

(57) ABSTRACT

A fluorinated ester selected from difluoromethyl 3,3,3-trifluoropropanoate or 2,2-difluoroethyl 3,3,3-trifluoropropanoate, methods of synthesizing the same, and uses of the same.

### CROSS REFERENCE TO RELATED APPLICATIONS

The present application claims the benefit of priority to U.S. Application Nos. 63/762,925, filed Feb. 25, 2025, 63/843,733, filed Jul. 14, 2025, 63/899,624, filed Oct. 15, 2025, and 63/988,883, filed Feb. 23, 2026, the contents of which in their entirety are herein incorporated by reference.

### FIELD

The present disclosure is directed to fluorinated esters, methods of synthesizing the same, and uses of the same, for example, incorporation into electrolytes for use in electrochemical energy storage devices.

### BACKGROUND

Electrochemical energy storage devices (also referred to as electrochemical cells or batteries) providing high charging voltage, wide operating temperature range, fast charge, and resistance to mechanical and thermal abuse, while exhibiting long cycle and calendar life are desired. Also desired are electrolytes for use in batteries that provide improved performance.

Further desired are compounds for inclusion in electrolytes for use in batteries that provide desirable characteristics to the batteries. Provided are compounds that provide desirable characteristics to electrolytes and batteries including such electrolytes.

### SUMMARY

The present disclosure provides a fluorinated ester selected from difluoromethyl 3,3,3-trifluoropropanoate or 2,2-difluoroethyl 3,3,3-trifluoropropanoate. 3,3,3-trifluoropropanoate is also known as 3,3,3-trifluoropropionate and 2,2-difluoroethyl 3,3,3-trifluoropropanoate is also known as 2,2-difluoroethyl 3,3,3-trifluoropropionate.

The present disclosure is based on the discovery that fluorinated esters difluoromethyl 3,3,3-trifluoropropanoate and 2,2-difluoroethyl 3,3,3-trifluoropropanoate provide beneficial properties to electrolytes and batteries including such electrolytes.

The present disclosure provides a method of synthesizing a fluorinated ester including (a) providing a carboxylic acid or carboxylic acid derivative; and (b) reacting the carboxylic acid or carboxylic acid derivative with: (i) difluorocarbene; or (ii) a fluorinated alcohol; to synthesize the fluorinated ester.

The present disclosure provides a method of synthesizing a fluorinated ester including (a) providing a fluorinated carboxylic acid or fluorinated carboxylic acid derivative; and (b) reacting the fluorinated carboxylic acid or fluorinated carboxylic acid derivative with: (i) difluorocarbene; or (ii) a fluorinated alcohol; to synthesize the fluorinated ester.

The present disclosure provides difluoromethyl 3,3,3-trifluoropropanoate and electrolytes including same.

The present disclosure provides 2,2-difluoroethyl 3,3,3-trifluoropropanoate and electrolytes including same.

The present disclosure provides a method of synthesizing difluoromethyl 3,3,3-trifluoropropanoate including (a) providing a fluorinated carboxylic acid; and (b) reacting the fluorinated carboxylic acid with difluorocarbene to synthesize the fluorinated ester.

The present disclosure provides a method of synthesizing difluoromethyl 3,3,3-trifluoropropanoate including (a) providing a fluorinated carboxylic acid; and (b) reacting the fluorinated carboxylic acid with difluorocarbene to synthesize the fluorinated ester.

The present disclosure provides a method of synthesizing 2,2-difluoroethyl 3,3,3-trifluoropropanoate including (a) providing a fluorinated acid fluoride; and (b) reacting the fluorinated acid fluoride with a fluorinated alcohol to synthesize the fluorinated ester.

The present disclosure provides a method of synthesizing 2,2-difluoroethyl 3,3,3-trifluoropropanoate including (a) providing a fluorinated acid halide; and (b) reacting the fluorinated acid halide with a fluorinated alcohol to synthesize the fluorinated ester. The fluorinated acid halide can be a fluorinated acid chloride. The fluorinated acid halide can be a fluorinated acid bromide.

The present disclosure provides an electrolyte including difluoromethyl 3,3,3-trifluoropropanoate, 2,2-difluoroethyl 3,3,3-trifluoropropanoate, or a combination thereof; and lithium bis(trifluoromethanesulfonyl)imide, lithium bis(fluorosulfonyl)imide, lithium bis(oxalato)borate, lithium hexafluorophosphate, lithium tetrafluoroborate, lithium perchlorate, lithium hexafluoroarsenate, lithium triflate, lithium nitrate, lithium difluorophosphate, lithium difluoro (oxalato) borate, lithium trifluoroacetate, lithium 4,5-dicyano-2-(trifluoromethyl)imidazole, lithium difluoro(dioxalato)phosphate, lithium tetrafluoro(oxalato)phosphate, lithium bis(pentafluoroethanesulfonyl)imide, or a combination thereof.

The present disclosure provides an electrolyte including difluoromethyl 3,3,3-trifluoropropanoate, 2,2-difluoroethyl 3,3,3-trifluoropropanoate, or a combination thereof; and sodium bis(trifluoromethanesulfonyl)imide, sodium bis(fluorosulfonyl)imide, sodium bis(oxalato)borate, sodium hexafluorophosphate, sodium tetrafluoroborate, sodium perchlorate, sodium hexafluoroarsenate, sodium triflate, sodium nitrate, sodium difluorophosphate, sodium difluoro(oxalato)borate, sodium trifluoroacetate, sodium 4,5-dicyano-2-(trifluoromethyl)imidazole, or a combination thereof.

The present disclosure provides an electrolyte including difluoromethyl 3,3,3-trifluoropropanoate, 2,2-difluoroethyl 3,3,3-trifluoropropanoate, or a combination thereof; fluoroethylene carbonate, propylene carbonate, dimethyl carbonate, diethyl carbonate, ethyl methyl carbonate, vinyl carbonate, vinyl ethylene carbonate, ethylene carbonate, difluoroethylene carbonate, 3,3,3-trifluoropropylene carbonate, monofluoroethyl methyl carbonate, difluoroethyl methyl carbonate, trifluoroethyl methyl carbonate, bis(2,2,2-trifluoroethyl) carbonate, 2,2-difluoroethyl ethyl carbonate, acetonitrile, fluoroacetonitrile, ethyl acetate, methyl acetate, methyl propanoate, methyl 3,3,3-trifluoropropanoate, ethyl 3,3,3-trifluoropropanoate, ethyl 2,2-difluoropropanoate, isopropyl trifluoroacetate, methyl 4,4,4-trifluorobutanoate, methyl difluoroacetate, 2,2,2-trifluoroethyl butanoate, methyl 2,2-difluoropropanoate, ethyl trifluoroacetate, 2,2-difluoroethyl trifluoroacetate, 2,2,2-trifluoroethyl acetate, ethyl 2,2-difluoroacetate, 2,2-difluoroethyl acetate, 2,2-difluoroethyl 3,3-difluoropropanoate, 2,2-difluoroethyl 2,2-difluoroacetate, succinonitrile, propionitrile, butyronitrile, adiponitrile, 1,3,6-hexanetricarbonitrile, trimethyl borate, triphenyl borate, triethyl borate, tris(pentafluorophenyl)borane, tris(trimethylsilyl)phosphate, tris(2,2,2-trifluoroethyl) borate, trimethyl phosphate, triethyl phosphate, tris(trimethylsilyl)phosphate, tris(trimethylsilyl)phosphite, tris(2,2,2-trifluoroethyl) phosphate, tris(2,2,2-trifluoroethyl) phosphite, (pentafluorophenyl)diphenyl phosphine, tris(pentafluorophenyl) phosphine, 1,3,2-dioxathiolane-2,2-dioxide, 1,3-propanesultone, prop-1-ene-1,3-sultone, propanediol cyclic sulfate, 1,3,2-dioxathiolane 2-oxide, bis(2,2,2-trifluoroethyl) ether, 1,1,2,2-tetrafluoroethyl-2,2,3,3-tetrafluoropropyl ether, 1H,1H,5H-octafluoropentyl-1,1,2,2-tetrafluoroethylether, 2,2,2-trifluoroethyl 1,1,2,2-tetrafluoroethyl ether, tetrahydrofuran, 1,3-dioxolane, 1,2-dimethoxyethane, diethylene glycol dimethyl ether, tetraethylene glycol dimethyl ether, succinic anhydride, butyric anhydride, 1,4-dioxane, tetravinyl silane, 1,4-butane sultone, dimethyl sulfoxide, methylene methanedisulfonate, N,N-dimethylformamide, gamma-butyrolactone, or a combination thereof; and a metal salt.

### BRIEF DESCRIPTION OF THE DRAWINGS

The above mentioned and other features of the disclosure, and the manner of attaining them, will become more apparent and the disclosure itself will be better understood by reference to the following description of embodiments of the disclosure taken in conjunction with the accompanying drawings.

FIG. 1 is a semi-schematic drawing of one example of a battery having an electrolyte of the present disclosure;

FIG. 2 is a graph of voltage (volts (V)) versus capacity (milliampere-hours (mAh)) of a first formation cycle of NMC811/graphite cell of Example 9C;

FIG. 3 is a graph of voltage (V) versus capacity (mAh) of a first formation cycle of a NMC811/graphite cell of Example 10C;

FIG. 4 is a graph of discharge capacity (mAh) versus cycle number for a NMC811/graphite cell of Example 10C and a comparative NMC811/graphite cell using a carbonate based electrolyte; and

FIG. 5 is a graph of capacity retention (percent (%)) versus cycle number for a NMC811/graphite cell of Example 10C and a comparative NMC811/graphite cell using a carbonate based electrolyte.

The exemplification set out herein illustrates an embodiment of the disclosure, and such exemplification is not to be construed as limiting the scope of the disclosure in any manner.

### DETAILED DESCRIPTION

### I. Definitions

TABLE 1
Names of compounds used herein and corresponding
structures and chemical names

| Name | Structure and Chemical Name |
|---|---|
| Fluorinated Ester 1 | 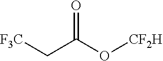 [SMILES: O=C(CC(F)(F)F)OC(F)F] |
| | difluoromethyl 3,3,3-trifluoropropanoate |
| Fluorinated Ester 2 | 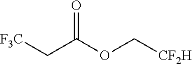 [SMILES: O=C(CC(F)(F)F)OCC(F)F] |
| | 2,2-difluoroethyl 3,3,3-trifluoropropanoate |

It is understood that the invention comprises, consists essentially of, or consists of the compounds identified in Table 1, alone or in combination.

As used herein, the singular forms “a”, “an”, and “the” include plural references unless the context clearly dictates otherwise. Thus, reference to “an” element in a claim followed by reference to “the” element is inclusive of one element and a plurality of the elements.

As used herein, the recitation of numerical ranges by endpoints includes all numbers subsumed within that range (e.g., 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the specification and attached listing of embodiments may vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

As used herein, the phrase “within any range encompassing any two of these values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

### II. Synthesis of Fluorinated Esters 1 and 2

The present disclosure includes methods of synthesizing fluorinated esters. A general method of synthesizing a fluorinated ester includes providing a carboxylic acid or carboxylic acid derivative; and reacting the carboxylic acid or carboxylic acid derivative with: (i) difluorocarbene; or (ii) a fluorinated alcohol; to synthesize the fluorinated ester. A general method of synthesizing a fluorinated ester includes providing a fluorinated carboxylic acid or fluorinated carboxylic acid derivative; and reacting the fluorinated carboxylic acid or fluorinated carboxylic acid derivative with: (i) difluorocarbene; or (ii) a fluorinated alcohol; to synthesize the fluorinated ester.

Fluorinated carboxylic acids and carboxylic acid derivatives are used. Suitable carboxylic acids include 3,3,3-trifluoropropanoic acid. Suitable fluorinated carboxylic acid derivatives include derivatives of 3,3,3-trifluoropropanoic acid. Fluorinated esters for synthesis by the methods disclosed herein include difluoromethyl 3,3,3-trifluoropropanoate (Fluorinated Ester 1) and 2,2-difluoroethyl 3,3,3-trifluoropropanoate (Fluorinated Ester 2).

A first synthetic route (“synthetic route 1”) for synthesizing a fluorinated ester includes providing a fluorinated carboxylic acid and reacting the fluorinated carboxylic acid with difluorocarbene to form a fluorinated ester. Synthetic example 1 disclosed herein describes synthesis of Fluorinated Ester 1 by synthetic route 1.

A second synthetic route (“synthetic route 2”) for synthesizing a fluorinated ester includes providing a fluorinated acid fluoride and reacting the fluorinated acid fluoride with a fluorinated alcohol to form a fluorinated ester. Synthetic example 2 disclosed herein describes synthesis of Fluorinated Ester 2 by synthetic route 2. An exemplary fluorinated acid fluoride is trifluoropropanoyl fluoride.

A third synthetic route (“synthetic route 3”) for synthesizing a fluorinated ester includes providing a fluorinated acid halide and reacting the fluorinated acid halide with a fluorinated alcohol to form a fluorinated ester. Synthetic example 7 disclosed herein describes synthesis of Fluorinated Ester 2 by synthetic route 3. The fluorinated acid halide can be a fluorinated acid chloride. An exemplary fluorinated acid chloride is trifluoropropanoyl chloride. The fluorinated acid halide can be a fluorinated acid bromide. An exemplary fluorinated acid bromide is trifluoropropanoyl bromide.

### III. Properties of Fluorinated Esters 1 and 2

Properties of Fluorinated Esters 1 and 2 including, dielectric constant, oxidation potential, dielectric constant, boiling point, and viscosity were calculated or simulated using computational methods. The properties suggest that Fluorinated Esters 1 and 2 will be beneficial as components of electrolytes for batteries, with different properties being more important than other properties, depending on a battery being formed and uses thereof. The properties of Fluorinated Esters 1 and 2 are provided in Table 2.

TABLE 2
Properties of Fluorinated Esters 1 and 2

| | Oxidation | | | Viscosity |
|---|---|---|---|---|
| | Potential | Dielectric | Boiling | at 25° C. |
| | (volts (V) | Constant | Point | (centipoise |
| Fluorinated Ester | vs. Li/Li⁺) | (Dk) | (° C.) | (cP)) |
| difluoromethyl 3,3,3- | 8.4 | 5.6 | 91 | 1.0 |
| trifluoropropanoate | | | | |
| (Fluorinated Ester 1) | | | | |
| 2,2-difluoroethyl 3,3,3- | 7.4 | 8.6 | 136 | 2.9 |
| trifluoropropanoate | | | | |
| (Fluorinated Ester 2) | | | | |

Oxidation potential is a measure of tendency to lose electrons and become oxidized at an electrode. Electrolytes and electrolyte components with high oxidation potentials can withstand high battery voltages without degrading via oxidation reactions. Oxidation potentials were computed using quantum chemistry for molecular dimers in implicit acetone solvent at the M05-2X/6-31+g(d,p) level of theory (Delp, Samuel A., Oleg Borodin, Marco Olguin, Claire G. Eisner, Joshua L. Allen, and T. Richard Jow, “Importance of reduction and oxidation stability of high voltage electrolytes and additives,” Electrochimica Acta 209 (2016): 498-510). Fluorinated Esters 1 and 2 have calculated oxidation potentials greater than 7 V vs. Li/Li⁺, suggesting usefulness in batteries with high operating voltages. Oxidation potentials can be measured using standard electrochemical techniques, including cyclic voltammetry or potentiostatic holds.

Dielectric constant (Dk) is a measure of ability to store electrical energy in an electric field. A higher dielectric constant of a solvent correlates with a higher ability of the solvent to dissolve salts, e.g., a higher solubility, for example, to form an electrolyte. Dielectric constants were computed using molecular dynamics simulations with the OpenFF 2.1.0 Sage force field (Wang, Lily, Pavan Kumar Behara, Matthew W. Thompson, Trevor Gokey, Yuanqing Wang, Jeffrey R. Wagner, Daniel J. Cole, Michael K. Gilson, Michael R. Shirts, and David L. Mobley, “The open force field initiative: Open software and open science for molecular modeling,” The Journal of Physical Chemistry B 128, no. 29 (2024): 7043-7067). Fluorinated Esters 1 and 2 have computed dielectric constants greater than 5, suggesting that Fluorinated Esters 1 and 2 will enable the dissolution of lithium or sodium salts to form an electrolyte. Dielectric constant can be measured using the open-ended coaxial probe technique, while the solubility of salts in a solvent can be measured using solvent addition or temperature variation methods.

Solvents for use in electrolytes have boiling points higher than the operating temperature of the battery to prevent gas generation within the battery. Boiling points were computed using COSMO-RS method as implemented in BIOVIA COSMOTherm (https://www.3ds.com/products/biovia/cosmo-rs/cosmotherm). Computed boiling points of Fluorinated Esters 1 and 2 are greater than or equal to 90° C., suggesting suitability as electrolyte components in battery applications operated at ambient temperature to greater than or equal to 80° C. Boiling points can be measured using an ebulliometer device, while gassing from batteries can be quantified using techniques based on Archimedes' principle.

Viscosity is a measure of resistance of a fluid to movement in neighboring portions relative to one another. Electrolyte viscosity impacts ion transport properties, with lower viscosity correlated to higher ionic conductivity and greater battery (dis)charge rate capability. As further disclosed herein, low viscosity allows ions within the electrolyte to move more freely, enhancing conductivity of the electrolyte, and improving operation of the electrolyte, and a battery including the same, at low temperatures. Viscosities were computed using QSPR models as implemented in BIOVIA COSMOTherm (https://www.3ds.com/products/biovia/cosmo-rs/cosmotherm). The computed viscosities of Fluorinated Esters 1 and 2 are less than or equal to 3 cP at 25° C., suggesting that electrolytes including Fluorinated Esters 1 and 2 will have suitable viscosity for lithium-ion and sodium-ion batteries. Viscosity can be measured using various types of viscometers, including capillary and rotational viscometers.

The number of fluorine atoms in a fluorinated ester affects flammability, with more fluorine atoms corresponding with desirable reduced flammability. The flammability of electrolytes can be characterized using standard techniques including open and closed cup flash point measurements.

### IV. Electrolytes

The present disclosure provides electrolytes including at least one of Fluorinated Esters 1 and 2 and a metal salt. The metal salt can be a lithium salt, a sodium salt, or a combination thereof.

### A. Metal Salt

### 1. Lithium Salt

The lithium salt can include lithium bis(trifluoromethanesulfonyl)imide (LiTFSI) (also referred to herein as Lithium Salt 1), lithium bis(fluorosulfonyl)imide (LIFSI) (also referred to herein as Lithium Salt 2), lithium bis(oxalato)borate (LiBOB) (also referred to herein as Lithium Salt 3), lithium hexafluorophosphate (LiPF₆) (also referred to herein as Lithium Salt 4), lithium tetrafluoroborate (LiBF₄) (also referred to herein as Lithium Salt 5), lithium perchlorate (LiCIO₄) (also referred to herein as Lithium Salt 6), lithium hexafluoroarsenate (LiAsF₆) (also referred to herein as Lithium Salt 7), lithium triflate (LiTf) (also referred to herein as Lithium Salt 8), lithium nitrate (LiNO₃) (also referred to herein as Lithium Salt 9), lithium difluorophosphate (LiDFP) (also referred to herein as Lithium Salt 10), lithium difluoro(oxalato)borate (LiDFOB) (also referred to herein as Lithium Salt 11), lithium trifluoroacetate (LiTFA) (also referred to herein as Lithium Salt 12), lithium 4,5-dicyano-2-(trifluoromethyl)imidazole (LiTDI) (also referred to herein as Lithium Salt 13), lithium difluoro(dioxalato)phosphate (LiDFDOP) (also referred to herein as Lithium Salt 14), lithium tetrafluoro(oxalato)phosphate (LiTFOP) (also referred to herein as Lithium Salt 15), lithium bis(pentafluoroethanesulfonyl)imide (LiBETI) (also referred to herein as Lithium Salt 16), or a combination thereof. The concentration of the lithium salt can be from about 0.01 moles per liter (molar, M) to about 5 M, from about 0.01 M to about 0.1 M, from about 0.3 M to about 2.5 M, from about 0.7 M to about 1.5 M, or within any range encompassed by any two of the foregoing values as endpoints. The concentration of lithium salt can be selected depending on the battery being formed and uses thereof.

Tables 3 and 4 define electrolytes including Fluorinated Ester 1 or Fluorinated Ester 2, respectively, and at least one lithium salt. The first column of each table identifies and defines the fluorinated ester (FE) by number as Fluorinated Ester 1 or Fluorinated Ester 2, and the Lithium Salt (LS) by number as Lithium Salt 1, Lithium Salt 2, etc. In the second column, the lithium salt is identified. The third column indicates the concentration of the lithium salt present in the electrolyte.

TABLE 3

| Fluorinated Ester | Lithium Salt | Lithium Salt Concentration |
|---|---|---|
| Fluorinated Ester 1 | LiTFSI | 0.01M-5M |
| Fluorinated Ester 1 | LiTFSI | 0.01M-0.1M |
| Fluorinated Ester 1 | LiTFSI | 0.3M-2.5M |
| Fluorinated Ester 1 | LiTFSI | 0.7M-1.5M |
| Fluorinated Ester 1 | LiFSI | 0.01M-5M |
| Fluorinated Ester 1 | LiFSI | 0.01M-0.1M |
| Fluorinated Ester 1 | LiFSI | 0.3M-2.5M |
| Fluorinated Ester 1 | LiFSI | 0.7M-1.5M |
| Fluorinated Ester 1 | LiBOB | 0.01M-5M |
| Fluorinated Ester 1 | LiBOB | 0.01M-0.1M |
| Fluorinated Ester 1 | LiBOB | 0.3M-2.5M |
| Fluorinated Ester 1 | LiBOB | 0.7M-1.5M |
| Fluorinated Ester 1 | LiPF₆ | 0.01M-5M |
| Fluorinated Ester 1 | LiPF₆ | 0.01M-0.1M |
| Fluorinated Ester 1 | LiPF₆ | 0.3M-2.5M |
| Fluorinated Ester 1 | LiPF₆ | 0.7M-1.5M |
| Fluorinated Ester 1 | LiBF₄ | 0.01M-5M |
| Fluorinated Ester 1 | LiBF₄ | 0.01M-0.1M |
| Fluorinated Ester 1 | LiBF₄ | 0.3M-2.5M |
| Fluorinated Ester 1 | LiBF₄ | 0.7M-1.5M |
| Fluorinated Ester 1 | LiClO₄ | 0.01M-5M |
| Fluorinated Ester 1 | LiClO₄ | 0.01M-0.1M |
| Fluorinated Ester 1 | LiClO₄ | 0.3M-2.5M |
| Fluorinated Ester 1 | LiClO₄ | 0.7M-1.5M |
| Fluorinated Ester 1 | LiASF₆ | 0.01M-5M |
| Fluorinated Ester 1 | LiASF₆ | 0.01M-0.1M |
| Fluorinated Ester 1 | LiASF₆ | 0.3M-2.5M |
| Fluorinated Ester 1 | LiASF₆ | 0.7M-1.5M |
| Fluorinated Ester 1 | LiTf | 0.01M-5M |
| Fluorinated Ester 1 | LiTf | 0.01M-0.1M |
| Fluorinated Ester 1 | LiTf | 0.3M-2.5M |
| Fluorinated Ester 1 | LiTf | 0.7M-1.5M |
| Fluorinated Ester 1 | LiNO₃ | 0.01M-5M |
| Fluorinated Ester 1 | LiNO₃ | 0.01M-0.1M |
| Fluorinated Ester 1 | LiNO₃ | 0.3M-2.5M |
| Fluorinated Ester 1 | LiNO₃ | 0.7M-1.5M |
| Fluorinated Ester 1 | LiDFP | 0.01M-5M |
| Fluorinated Ester 1 | LiDFP | 0.01M-0.1M |
| Fluorinated Ester 1 | LiDFP | 0.3M-2.5M |
| Fluorinated Ester 1 | LiDFP | 0.7M-1.5M |
| Fluorinated Ester 1 | LiDFOB | 0.01M-5M |
| Fluorinated Ester 1 | LiDFOB | 0.01M-0.1M |
| Fluorinated Ester 1 | LiDFOB | 0.3M-2.5M |
| Fluorinated Ester 1 | LiDFOB | 0.7M-1.5M |
| Fluorinated Ester 1 | LiTFA | 0.01M-5M |
| Fluorinated Ester 1 | LiTFA | 0.01M-0.1M |
| Fluorinated Ester 1 | LiTFA | 0.3M-2.5M |
| Fluorinated Ester 1 | LiTFA | 0.7M-1.5M |
| Fluorinated Ester 1 | LiTDI | 0.01M-5M |
| Fluorinated Ester 1 | LiTDI | 0.01M-0.1M |
| Fluorinated Ester 1 | LiTDI | 0.3M-2.5M |
| Fluorinated Ester 1 | LiTDI | 0.7M-1.5M |
| Fluorinated Ester 1 | LiDFDOP | 0.01M-5M |
| Fluorinated Ester 1 | LiDFDOP | 0.01M-0.1M |
| Fluorinated Ester 1 | LiDFDOP | 0.3M-2.5M |
| Fluorinated Ester 1 | LiDFDOP | 0.7M-1.5M |
| Fluorinated Ester 1 | LiTFOP | 0.01M-5M |
| Fluorinated Ester 1 | LiTFOP | 0.01M-0.1M |
| Fluorinated Ester 1 | LiTFOP | 0.3M-2.5M |
| Fluorinated Ester 1 | LiTFOP | 0.7M-1.5M |
| Fluorinated Ester 1 | LiBETI | 0.01M-5M |
| Fluorinated Ester 1 | LiBETI | 0.01M-0.1M |
| Fluorinated Ester 1 | LiBETI | 0.3M-2.5M |
| Fluorinated Ester 1 | LiBETI | 0.7M-1.5M |

TABLE 4

| Fluorinated Ester | Lithium Salt | Lithium Salt Concentration |
|---|---|---|
| Fluorinated Ester 2 | LiTFSI | 0.01M-5M |
| Fluorinated Ester 2 | LiTFSI | 0.01M-0.1M |
| Fluorinated Ester 2 | LiTFSI | 0.3M-2.5M |
| Fluorinated Ester 2 | LiTFSI | 0.7M-1.5M |
| Fluorinated Ester 2 | LiFSI | 0.01M-5M |
| Fluorinated Ester 2 | LiFSI | 0.01M-0.1M |
| Fluorinated Ester 2 | LiFSI | 0.3M-2.5M |
| Fluorinated Ester 2 | LiFSI | 0.7M-1.5M |
| Fluorinated Ester 2 | LiBOB | 0.01M-5M |
| Fluorinated Ester 2 | LiBOB | 0.01M-0.1M |
| Fluorinated Ester 2 | LiBOB | 0.3M-2.5M |
| Fluorinated Ester 2 | LiBOB | 0.7M-1.5M |
| Fluorinated Ester 2 | LiPF₆ | 0.01M-5M |
| Fluorinated Ester 2 | LiPF₆ | 0.01M-0.1M |
| Fluorinated Ester 2 | LiPF₆ | 0.3M-2.5M |
| Fluorinated Ester 2 | LiPF₆ | 0.7M-1.5M |
| Fluorinated Ester 2 | LiBF₄ | 0.01M-5M |
| Fluorinated Ester 2 | LiBF₄ | 0.01M-0.1M |
| Fluorinated Ester 2 | LiBF₄ | 0.3M-2.5M |
| Fluorinated Ester 2 | LiBF₄ | 0.7M-1.5M |
| Fluorinated Ester 2 | LiClO₄ | 0.01M-5M |
| Fluorinated Ester 2 | LiClO₄ | 0.01M-0.1M |
| Fluorinated Ester 2 | LiClO₄ | 0.3M-2.5M |
| Fluorinated Ester 2 | LiClO₄ | 0.7M-1.5M |
| Fluorinated Ester 2 | LiASF₆ | 0.01M-5M |
| Fluorinated Ester 2 | LiASF₆ | 0.01M-0.1M |
| Fluorinated Ester 2 | LiASF₆ | 0.3M-2.5M |
| Fluorinated Ester 2 | LiASF₆ | 0.7M-1.5M |
| Fluorinated Ester 2 | LiTf | 0.01M-5M |
| Fluorinated Ester 2 | LiTf | 0.01M-0.1M |
| Fluorinated Ester 2 | LiTf | 0.3M-2.5M |
| Fluorinated Ester 2 | LiTf | 0.7M-1.5M |
| Fluorinated Ester 2 | LiNO₃ | 0.01M-5M |
| Fluorinated Ester 2 | LiNO₃ | 0.01M-0.1M |
| Fluorinated Ester 2 | LiNO₃ | 0.3M-2.5M |
| Fluorinated Ester 2 | LiNO₃ | 0.7M-1.5M |
| Fluorinated Ester 2 | LiDFP | 0.01M-5M |
| Fluorinated Ester 2 | LiDFP | 0.01M-0.1M |
| Fluorinated Ester 2 | LiDFP | 0.3M-2.5M |
| Fluorinated Ester 2 | LiDFP | 0.7M-1.5M |
| Fluorinated Ester 2 | LiDFOB | 0.01M-5M |
| Fluorinated Ester 2 | LiDFOB | 0.01M-0.1M |
| Fluorinated Ester 2 | LiDFOB | 0.3M-2.5M |
| Fluorinated Ester 2 | LiDFOB | 0.7M-1.5M |
| Fluorinated Ester 2 | LiTFA | 0.01M-5M |
| Fluorinated Ester 2 | LiTFA | 0.01M-0.1M |
| Fluorinated Ester 2 | LiTFA | 0.3M-2.5M |
| Fluorinated Ester 2 | LiTFA | 0.7M-1.5M |
| Fluorinated Ester 2 | LiTDI | 0.01M-5M |
| Fluorinated Ester 2 | LiTDI | 0.01M-0.1M |
| Fluorinated Ester 2 | LiTDI | 0.3M-2.5M |
| Fluorinated Ester 2 | LiTDI | 0.7M-1.5M |
| Fluorinated Ester 2 | LiDFDOP | 0.01M-5M |
| Fluorinated Ester 2 | LiDFDOP | 0.01M-0.1M |
| Fluorinated Ester 2 | LiDFDOP | 0.3M-2.5M |
| Fluorinated Ester 2 | LiDFDOP | 0.7M-1.5M |
| Fluorinated Ester 2 | LiTFOP | 0.01M-5M |
| Fluorinated Ester 2 | LiTFOP | 0.01M-0.1M |
| Fluorinated Ester 2 | LiTFOP | 0.3M-2.5M |
| Fluorinated Ester 2 | LiTFOP | 0.7M-1.5M |
| Fluorinated Ester 2 | LiBETI | 0.01M-5M |
| Fluorinated Ester 2 | LiBETI | 0.01M-0.1M |
| Fluorinated Ester 2 | LiBETI | 0.3M-2.5M |
| Fluorinated Ester 2 | LiBETI | 0.7M-1.5M |

It is understood that the invention comprises, consists essentially of, or consists of the compositions identified in Table 3. It is understood that the invention comprises, consists essentially of, or consists of the compositions identified in Table 4.

The electrolyte can include more than one lithium salt, for example, two lithium salts, three lithium salts, or four or more lithium salts. Exemplary combinations of more than one lithium salt include, for example, LiFSI and LiPF₆; or LiDFOB and one or more of LiPF₆, LiTFSI, LiFSI, or LiBF₄. With reference to Tables 3 and 4, when a listed electrolyte further includes one or more additional lithium salt(s) in addition to the listed lithium salt, the listed lithium salt concentration can refer to the total lithium salt concentration or the lithium salt concentration of each included lithium salt. A combination of lithium salts, concentrations thereof, or a combination thereof can be selected depending on a battery being formed and uses thereof.

### 2. Sodium Salts

The sodium salt can include sodium bis(trifluoromethanesulfonyl)imide (NaTFSI) (also referred to herein as Sodium Salt 1), sodium bis(fluorosulfonyl)imide (NaFSI) (also referred to herein as Sodium Salt 2), sodium bis(oxalato)borate (NaBOB) (also referred to herein as Sodium Salt 3), sodium hexafluorophosphate (NaPF₆) (also referred to herein as Sodium Salt 4), sodium tetrafluoroborate (NaBF4) (also referred to herein as Sodium Salt 5), sodium perchlorate (NaClO₄) (also referred to herein as Sodium Salt 6), sodium hexafluoroarsenate (NaAsF₆) (also referred to herein as Sodium Salt 7), sodium triflate (NaTf) (also referred to herein as Sodium Salt 8), sodium nitrate (NaNO₃) (also referred to herein as Sodium Salt 9), sodium difluorophosphate (NaDFP) (also referred to herein as Sodium Salt 10), sodium difluoro (oxalato) borate (NaDFOB) (also referred to herein as Sodium Salt 11), sodium trifluoroacetate (NaTFA) (also referred to herein as Sodium Salt 12), sodium 4,5-dicyano-2-(trifluoromethyl)imidazole (NaTDI) (also referred to herein as Sodium Salt 13), or a combination thereof. The concentration of the sodium salt can be from about 0.01 M to about 5 M, from about 0.01 M to about 0.1 M, from about 0.3 M to about 2.5 M, from about 0.7 M to about 1.5 M, or within any range encompassed by any two of the foregoing values as endpoints.

Tables 5 and 6 define electrolytes including Fluorinated Ester 1 or Fluorinated Ester 2, respectively, and at least one sodium salt. The first column of each table identifies and defines the fluorinated ester (FE) by number as Fluorinated Ester 1 or Fluorinated Ester 2, and the Sodium Salt (SS) by number as Sodium Salt 1, Sodium Salt 2, etc. In the second column, the sodium salt is identified. The third column indicates the concentration of the sodium salt present in the electrolyte.

TABLE 5

| Fluorinated Ester | Sodium Salt | Sodium Salt Concentration |
|---|---|---|
| Fluorinated Ester 1 | NaTFSI | 0.01M-5M |
| Fluorinated Ester 1 | NaTFSI | 0.01M-0.1M |
| Fluorinated Ester 1 | NaTFSI | 0.3M-2.5M |
| Fluorinated Ester 1 | NaTFSI | 0.7M-1.5M |
| Fluorinated Ester 1 | NaFSI | 0.01M-5M |
| Fluorinated Ester 1 | NaFSI | 0.01M-0.1M |
| Fluorinated Ester 1 | NaFSI | 0.3M-2.5M |
| Fluorinated Ester 1 | NaFSI | 0.7M-1.5M |
| Fluorinated Ester 1 | NaBOB | 0.01M-5M |
| Fluorinated Ester 1 | NaBOB | 0.01M-0.1M |
| Fluorinated Ester 1 | NaBOB | 0.3M-2.5M |
| Fluorinated Ester 1 | NaBOB | 0.7M-1.5M |
| Fluorinated Ester 1 | NaPF₆ | 0.01M-5M |
| Fluorinated Ester 1 | NaPF₆ | 0.01M-0.1M |
| Fluorinated Ester 1 | NaPF₆ | 0.3M-2.5M |
| Fluorinated Ester 1 | NaPF₆ | 0.7M-1.5M |
| Fluorinated Ester 1 | NaBF₄ | 0.01M-5M |
| Fluorinated Ester 1 | NaBF₄ | 0.01M-0.1M |
| Fluorinated Ester 1 | NaBF₄ | 0.3M-2.5M |
| Fluorinated Ester 1 | NaBF₄ | 0.7M-1.5M |
| Fluorinated Ester 1 | NaClO₄ | 0.01M-5M |
| Fluorinated Ester 1 | NaClO₄ | 0.01M-0.1M |
| Fluorinated Ester 1 | NaClO₄ | 0.3M-2.5M |
| Fluorinated Ester 1 | NaClO₄ | 0.7M-1.5M |
| Fluorinated Ester 1 | NaAsF₆ | 0.01M-5M |
| Fluorinated Ester 1 | NaAsF₆ | 0.01M-0.1M |
| Fluorinated Ester 1 | NaAsF₆ | 0.3M-2.5M |
| Fluorinated Ester 1 | NaAsF₆ | 0.7M-1.5M |
| Fluorinated Ester 1 | NaTf | 0.01M-5M |
| Fluorinated Ester 1 | NaTf | 0.01M-0.1M |
| Fluorinated Ester 1 | NaTf | 0.3M-2.5M |
| Fluorinated Ester 1 | NaTf | 0.7M-1.5M |
| Fluorinated Ester 1 | NaNO₃ | 0.01M-5M |
| Fluorinated Ester 1 | NaNO₃ | 0.01M-0.1M |
| Fluorinated Ester 1 | NaNO₃ | 0.3M-2.5M |
| Fluorinated Ester 1 | NaNO₃ | 0.7M-1.5M |
| Fluorinated Ester 1 | NaDFP | 0.01M-5M |
| Fluorinated Ester 1 | NaDFP | 0.01M-0.1M |
| Fluorinated Ester 1 | NaDFP | 0.3M-2.5M |
| Fluorinated Ester 1 | NaDFP | 0.7M-1.5M |
| Fluorinated Ester 1 | NaDFOB | 0.01M-5M |
| Fluorinated Ester 1 | NaDFOB | 0.01M-0.1M |
| Fluorinated Ester 1 | NaDFOB | 0.3M-2.5M |
| Fluorinated Ester 1 | NaDFOB | 0.7M-1.5M |
| Fluorinated Ester 1 | NaTFA | 0.01M-5M |
| Fluorinated Ester 1 | NaTFA | 0.01M-0.1M |
| Fluorinated Ester 1 | NaTFA | 0.3M-2.5M |
| Fluorinated Ester 1 | NaTFA | 0.7M-1.5M |
| Fluorinated Ester 1 | NaTDI | 0.01M-5M |
| Fluorinated Ester 1 | NaTDI | 0.01M-0.1M |
| Fluorinated Ester 1 | NaTDI | 0.3M-2.5M |
| Fluorinated Ester 1 | NaTDI | 0.7M-1.5M |

TABLE 6

| Fluorinated Ester | Sodium Salt | Sodium Salt Concentration |
|---|---|---|
| Fluorinated Ester 2 | NaTFSI | 0.01M-5M |
| Fluorinated Ester 2 | NaTFSI | 0.01M-0.1M |
| Fluorinated Ester 2 | NaTFSI | 0.3M-2.5M |
| Fluorinated Ester 2 | NaTFSI | 0.7M-1.5M |
| Fluorinated Ester 2 | NaFSI | 0.01M-5M |
| Fluorinated Ester 2 | NaFSI | 0.01M-0.1M |
| Fluorinated Ester 2 | NaFSI | 0.3M-2.5M |
| Fluorinated Ester 2 | NaFSI | 0.7M-1.5M |
| Fluorinated Ester 2 | NaBOB | 0.01M-5M |
| Fluorinated Ester 2 | NaBOB | 0.01M-0.1M |
| Fluorinated Ester 2 | NaBOB | 0.3M-2.5M |
| Fluorinated Ester 2 | NaBOB | 0.7M-1.5M |
| Fluorinated Ester 2 | NaPF₆ | 0.01M-5M |
| Fluorinated Ester 2 | NaPF₆ | 0.01M-0.1M |
| Fluorinated Ester 2 | NaPF₆ | 0.3M-2.5M |
| Fluorinated Ester 2 | NaPF₆ | 0.7M-1.5M |
| Fluorinated Ester 2 | NaBF₄ | 0.01M-5M |
| Fluorinated Ester 2 | NaBF₄ | 0.01M-0.1M |
| Fluorinated Ester 2 | NaBF₄ | 0.3M-2.5M |
| Fluorinated Ester 2 | NaBF₄ | 0.7M-1.5M |
| Fluorinated Ester 2 | NaClO₄ | 0.01M-5M |
| Fluorinated Ester 2 | NaClO₄ | 0.01M-0.1M |
| Fluorinated Ester 2 | NaClO₄ | 0.3M-2.5M |
| Fluorinated Ester 2 | NaClO₄ | 0.7M-1.5M |
| Fluorinated Ester 2 | NaAsF₆ | 0.01M-5M |
| Fluorinated Ester 2 | NaAsF₆ | 0.01M-0.1M |
| Fluorinated Ester 2 | NaAsF₆ | 0.3M-2.5M |
| Fluorinated Ester 2 | NaAsF₆ | 0.7M-1.5M |
| Fluorinated Ester 2 | NaTf | 0.01M-5M |
| Fluorinated Ester 2 | NaTf | 0.01M-0.1M |
| Fluorinated Ester 2 | NaTf | 0.3M-2.5M |
| Fluorinated Ester 2 | NaTf | 0.7M-1.5M |
| Fluorinated Ester 2 | NaNO₃ | 0.01M-5M |
| Fluorinated Ester 2 | NaNO₃ | 0.01M-0.1M |
| Fluorinated Ester 2 | NaNO₃ | 0.3M-2.5M |
| Fluorinated Ester 2 | NaNO₃ | 0.7M-1.5M |
| Fluorinated Ester 2 | NaDFP | 0.01M-5M |
| Fluorinated Ester 2 | NaDFP | 0.01M-0.1M |
| Fluorinated Ester 2 | NaDFP | 0.3M-2.5M |
| Fluorinated Ester 2 | NaDFP | 0.7M-1.5M |
| Fluorinated Ester 2 | NaDFOB | 0.01M-5M |
| Fluorinated Ester 2 | NaDFOB | 0.01M-0.1M |
| Fluorinated Ester 2 | NaDFOB | 0.3M-2.5M |
| Fluorinated Ester 2 | NaDFOB | 0.7M-1.5M |
| Fluorinated Ester 2 | NaTFA | 0.01M-5M |
| Fluorinated Ester 2 | NaTFA | 0.01M-0.1M |
| Fluorinated Ester 2 | NaTFA | 0.3M-2.5M |
| Fluorinated Ester 2 | NaTFA | 0.7M-1.5M |
| Fluorinated Ester 2 | NaTDI | 0.01M-5M |
| Fluorinated Ester 2 | NaTDI | 0.01M-0.1M |
| Fluorinated Ester 2 | NaTDI | 0.3M-2.5M |
| Fluorinated Ester 2 | NaTDI | 0.7M-1.5M |

It is understood that the invention comprises, consists essentially of, or consists of the compositions identified in Table 5. It is understood that the invention comprises, consists essentially of, or consists of the compositions identified in Table 6.

The electrolyte can include more than one sodium salt, for example, two sodium salts, three sodium salts, or four or more sodium salts. Exemplary combinations of more than one sodium salt include, for example, NaFSI and NaPF₆; or NaDFOB and one or more of NaPF₆, NaTFSI, NaFSI, or NaBF₄. With reference to Tables 5 and 6, when a listed electrolyte further includes one or more additional sodium salt(s) in addition to the listed sodium salt, the listed sodium salt concentration can refer to the total sodium salt concentration or the sodium salt concentration of each included sodium salt. A combination of sodium salts, concentrations thereof, or a combination thereof can be selected depending on a battery being formed and uses thereof.

### B. Supplemental Solvent

Electrolytes including at least one of Fluorinated Esters 1 and 2 can include a supplemental solvent, e.g., at least one, solvent that is not one of Fluorinated Esters 1 and 2. Exemplary reasons for including Fluorinated Esters 1 and 2, at least one supplemental solvent, or a combination thereof in an electrolyte include to adjust electrolyte viscosity, to improve ability to solvate, to form an interphase at a cathode or anode of a battery including the electrolyte, to function as a hydrofluoric acid scavenger, and to function as a safety additive.

For example, an electrolyte including at least one of Fluorinated Esters 1 and 2, at least one supplemental solvent, or a combination thereof can allow for a lower viscosity of the electrolyte, for example, at low temperatures, than would be exhibited by an electrolyte lacking at least one of Fluorinated Esters 1 and 2, at least one supplemental solvent, or a combination thereof. A lower viscosity allows ions within the electrolyte to move more freely, enhancing conductivity of the electrolyte.

Fluorinated Esters 1 and 2, at least one supplemental solvent, or a combination thereof can serve as a film-forming additive that can create a protective film on a surface of an electrode of a battery during operation of the battery. The film can improve performance, e.g., cycling performance, of the battery by stabilizing an interface between the electrolyte and the electrode. During the first charging cycle, the film-forming additive may decompose and deposit an interphase layer, called a solid electrolyte interphase (SEI) on the anode, and called a cathode electrolyte interphase (CEI) on the cathode. The SEI, CEI, or a combination thereof can prevent the electrolyte from directly contacting the charged electrodes.

Tables 7A and 7B includes such solvents that are not one of Fluorinated Esters 1 and 2 that can be included in the electrolyte. The amount of supplemental solvent in the electrolyte can be from about 0.5 wt. % to about 99.0 wt. %, based on a total weight of the electrolyte. Fluorinated Esters 1 and 2 can be present in an amount of from 0.1 wt. % to 5 wt. % (e.g., as an additive), from greater than 5 wt. % to 25 wt. % (e.g., as a secondary solvent), or from greater than 25 wt. % to 90 wt. % (e.g., as a primary solvent), or within any range encompassed by any two of the foregoing values as endpoints, based on a total weight of the electrolyte. The amount of Fluorinated Esters 1 and 2 can be selected depending on a battery being formed and uses thereof. It is understood that the invention comprises, consists essentially of, or consists of the compositions identified in Table 7A. It is understood that the invention comprises, consists essentially of, or consists of the compositions identified in Table 7B.

TABLE 7A

| | | Concentration of |
|---|---|---|
| Fluorinated Ester | Solvent | Fluorinated Ester 1 |
| Fluorinated Ester 1 | fluoroethylene carbonate (FEC) | 0.1 wt. %-5 wt. % |
| Fluorinated Ester 1 | fluoroethylene carbonate (FEC) | >5 wt. %-25 wt. % |
| Fluorinated Ester 1 | fluoroethylene carbonate (FEC) | >25 wt. %-95 wt. % |
| Fluorinated Ester 1 | propylene carbonate (PC) | 0.1 wt. %-5 wt. % |
| Fluorinated Ester 1 | propylene carbonate (PC) | >5 wt. %-25 wt. % |
| Fluorinated Ester 1 | propylene carbonate (PC) | >25 wt. %-95 wt. % |
| Fluorinated Ester 1 | dimethyl carbonate (DMC) | 0.1 wt. %-5 wt. % |
| Fluorinated Ester 1 | dimethyl carbonate (DMC) | >5 wt. %-25 wt. % |
| Fluorinated Ester 1 | dimethyl carbonate (DMC) | >25 wt. %-95 wt. % |
| Fluorinated Ester 1 | diethyl carbonate (DEC) | 0.1 wt. %-5 wt. % |
| Fluorinated Ester 1 | diethyl carbonate (DEC) | >5 wt. %-25 wt. % |
| Fluorinated Ester 1 | diethyl carbonate (DEC) | >25 wt. %-95 wt. % |
| Fluorinated Ester 1 | ethyl methyl carbonate (EMC) | 0.1 wt. %-5 wt. % |
| Fluorinated Ester 1 | ethyl methyl carbonate (EMC) | >5 wt. %-25 wt. % |
| Fluorinated Ester 1 | ethyl methyl carbonate (EMC) | >25 wt. %-95 wt. % |
| Fluorinated Ester 1 | vinyl carbonate (VC) | 0.1 wt. %-5 wt. % |
| Fluorinated Ester 1 | vinyl carbonate (VC) | >5 wt. %-25 wt. % |
| Fluorinated Ester 1 | vinyl carbonate (VC) | >25 wt. %-95 wt. % |
| Fluorinated Ester 1 | vinyl ethylene carbonate (VEC) | 0.1 wt. %-5 wt. % |
| Fluorinated Ester 1 | vinyl ethylene carbonate (VEC) | >5 wt. %-25 wt. % |
| Fluorinated Ester 1 | vinyl ethylene carbonate (VEC) | >25 wt. %-95 wt. % |
| Fluorinated Ester 1 | ethylene carbonate (EC) | 0.1 wt. %-5 wt. % |
| Fluorinated Ester 1 | ethylene carbonate (EC) | >5 wt. %-25 wt. % |
| Fluorinated Ester 1 | ethylene carbonate (EC) | >25 wt. %-95 wt. % |
| Fluorinated Ester 1 | difluoroethylene carbonate (DFEC) | 0.1 wt. %-5 wt. % |
| Fluorinated Ester 1 | difluoroethylene carbonate (DFEC) | >5 wt. %-25 wt. % |
| Fluorinated Ester 1 | difluoroethylene carbonate (DFEC) | >25 wt. %-95 wt. % |
| Fluorinated Ester 1 | 3,3,3-trifluoropropylene carbonate | 0.1 wt. %-5 wt. % |
| | (TFPC) | |
| Fluorinated Ester 1 | 3,3,3-trifluoropropylene carbonate | >5 wt. %-25 wt. % |
| | (TFPC) | |
| Fluorinated Ester 1 | 3,3,3-trifluoropropylene carbonate | >25 wt. %-95 wt. % |
| | (TFPC) | |
| Fluorinated Ester 1 | monofluoroethyl methyl carbonate | 0.1 wt. %-5 wt. % |
| | (F1EMC) | |
| Fluorinated Ester 1 | monofluoroethyl methyl carbonate | >5 wt. %-25 wt. % |
| | (F1EMC) | |
| Fluorinated Ester 1 | monofluoroethyl methyl carbonate | >25 wt. %-95 wt. % |
| | (F1EMC) | |
| Fluorinated Ester 1 | difluoroethyl methyl carbonate (F2EMC) | 0.1 wt. %-5 wt. % |
| Fluorinated Ester 1 | difluoroethyl methyl carbonate (F2EMC) | >5 wt. %-25 wt. % |
| Fluorinated Ester 1 | difluoroethyl methyl carbonate (F2EMC) | >25 wt. %-95 wt. % |
| Fluorinated Ester 1 | trifluoroethyl methyl carbonate (F3EMC) | 0.1 wt. %-5 wt. % |
| Fluorinated Ester 1 | trifluoroethyl methyl carbonate (F3EMC) | >5 wt. %-25 wt. % |
| Fluorinated Ester 1 | trifluoroethyl methyl carbonate (F3EMC) | >25 wt. %-95 wt. % |
| Fluorinated Ester 1 | bis(2,2,2-trifluoroethyl) carbonate | 0.1 wt. %-5 wt. % |
| | (TFEC) | |
| Fluorinated Ester 1 | bis(2,2,2-trifluoroethyl) carbonate | >5 wt. %-25 wt. % |
| | (TFEC) | |
| Fluorinated Ester 1 | bis(2,2,2-trifluoroethyl) carbonate | >25 wt. %-95 wt. % |
| | (TFEC) | |
| Fluorinated Ester 1 | 2,2-difluoroethyl ethyl carbonate | 0.1 wt. %-5 wt. % |
| | (DFDEC) | |
| Fluorinated Ester 1 | 2,2-difluoroethyl ethyl carbonate | >5 wt. %-25 wt. % |
| | (DFDEC) | |
| Fluorinated Ester 1 | 2,2-difluoroethyl ethyl carbonate | >25 wt. %-95 wt. % |
| | (DFDEC) | |
| Fluorinated Ester 1 | acetonitrile (AN) | 0.1 wt. %-5 wt. % |
| Fluorinated Ester 1 | acetonitrile (AN) | >5 wt. %-25 wt. % |
| Fluorinated Ester 1 | acetonitrile (AN) | >25 wt. %-95 wt. % |
| Fluorinated Ester 1 | fluoroacetonitrile | 0.1 wt. %-5 wt. % |
| Fluorinated Ester 1 | fluoroacetonitrile | >5 wt. %-25 wt. % |
| Fluorinated Ester 1 | fluoroacetonitrile | >25 wt. %-95 wt. % |
| Fluorinated Ester 1 | ethyl acetate (EA) | 0.1 wt. %-5 wt. % |
| Fluorinated Ester 1 | ethyl acetate (EA) | >5 wt. %-25 wt. % |
| Fluorinated Ester 1 | ethyl acetate (EA) | >25 wt. %-95 wt. % |
| Fluorinated Ester 1 | methyl acetate (MA) | 0.1 wt. %-5 wt. % |
| Fluorinated Ester 1 | methyl acetate (MA) | >5 wt. %-25 wt. % |
| Fluorinated Ester 1 | methyl acetate (MA) | >25 wt. %-95 wt. % |
| Fluorinated Ester 1 | methyl propanoate (MP) | 0.1 wt. %-5 wt. % |
| Fluorinated Ester 1 | methyl propanoate (MP) | >5 wt. %-25 wt. % |
| Fluorinated Ester 1 | methyl propanoate (MP) | >25 wt. %-95 wt. % |
| Fluorinated Ester 1 | methyl 3,3,3-trifluoropropanoate | 0.1 wt. %-5 wt. % |
| Fluorinated Ester 1 | methyl 3,3,3-trifluoropropanoate | >5 wt. %-25 wt. % |
| Fluorinated Ester 1 | methyl 3,3,3-trifluoropropanoate | >25 wt. %-95 wt. % |
| Fluorinated Ester 1 | ethyl 3,3,3-trifluoropropanoate | 0.1 wt. %-5 wt. % |
| Fluorinated Ester 1 | ethyl 3,3,3-trifluoropropanoate | >5 wt. %-25 wt. % |
| Fluorinated Ester 1 | ethyl 3,3,3-trifluoropropanoate | >25 wt. %-95 wt. % |
| Fluorinated Ester 1 | ethyl 2,2-difluoropropanoate | 0.1 wt. %-5 wt. % |
| Fluorinated Ester 1 | ethyl 2,2-difluoropropanoate | >5 wt. %-25 wt. % |
| Fluorinated Ester 1 | ethyl 2,2-difluoropropanoate | >25 wt. %-95 wt. % |
| Fluorinated Ester 1 | isopropyl trifluoroacetate | 0.1 wt. %-5 wt. % |
| Fluorinated Ester 1 | isopropyl trifluoroacetate | >5 wt. %-25 wt. % |
| Fluorinated Ester 1 | isopropyl trifluoroacetate | >25 wt. %-95 wt. % |
| Fluorinated Ester 1 | methyl 4,4,4-trifluorobutanoate | 0.1 wt. %-5 wt. % |
| Fluorinated Ester 1 | methyl 4,4,4-trifluorobutanoate | >5 wt. %-25 wt. % |
| Fluorinated Ester 1 | methyl 4,4,4-trifluorobutanoate | >25 wt. %-95 wt. % |
| Fluorinated Ester 1 | methyl difluoroacetate | 0.1 wt. %-5 wt. % |
| Fluorinated Ester 1 | methyl difluoroacetate | >5 wt. %-25 wt. % |
| Fluorinated Ester 1 | methyl difluoroacetate | >25 wt. %-95 wt. % |
| Fluorinated Ester 1 | 2,2,2-trifluoroethyl butanoate | 0.1 wt. %-5 wt. % |
| Fluorinated Ester 1 | 2,2,2-trifluoroethyl butanoate | >5 wt. %-25 wt. % |
| Fluorinated Ester 1 | 2,2,2-trifluoroethyl butanoate | >25 wt. %-95 wt. % |
| Fluorinated Ester 1 | methyl 2,2-difluoropropanoate | 0.1 wt. %-5 wt. % |
| Fluorinated Ester 1 | methyl 2,2-difluoropropanoate | >5 wt. %-25 wt. % |
| Fluorinated Ester 1 | methyl 2,2-difluoropropanoate | >25 wt. %-95 wt. % |
| Fluorinated Ester 1 | ethyl trifluoroacetate | 0.1 wt. %-5 wt. % |
| Fluorinated Ester 1 | ethyl trifluoroacetate | >5 wt. %-25 wt. % |
| Fluorinated Ester 1 | ethyl trifluoroacetate | >25 wt. %-95 wt. % |
| Fluorinated Ester 1 | 2,2-difluoroethyl trifluoroacetate | 0.1 wt. %-5 wt. % |
| Fluorinated Ester 1 | 2,2-difluoroethyl trifluoroacetate | >5 wt. %-25 wt. % |
| Fluorinated Ester 1 | 2,2-difluoroethyl trifluoroacetate | >25 wt. %-95 wt. % |
| Fluorinated Ester 1 | 2,2,2-trifluoroethyl acetate | 0.1 wt. %-5 wt. % |
| Fluorinated Ester 1 | 2,2,2-trifluoroethyl acetate | >5 wt. %-25 wt. % |
| Fluorinated Ester 1 | 2,2,2-trifluoroethyl acetate | >25 wt. %-95 wt. % |
| Fluorinated Ester 1 | ethyl 2,2-difluoroacetate | 0.1 wt. %-5 wt. % |
| Fluorinated Ester 1 | ethyl 2,2-difluoroacetate | >5 wt. %-25 wt. % |
| Fluorinated Ester 1 | ethyl 2,2-difluoroacetate | >25 wt. %-95 wt. % |
| Fluorinated Ester 1 | 2,2-difluoroethyl acetate | 0.1 wt. %-5 wt. % |
| Fluorinated Ester 1 | 2,2-difluoroethyl acetate | >5 wt. %-25 wt. % |
| Fluorinated Ester 1 | 2,2-difluoroethyl acetate | >25 wt. %-95 wt. % |
| Fluorinated Ester 1 | 2,2-difluoroethyl 3,3-difluoropropanoate | 0.1 wt. %-5 wt. % |
| Fluorinated Ester 1 | 2,2-difluoroethyl 3,3-difluoropropanoate | >5 wt. %-25 wt. % |
| Fluorinated Ester 1 | 2,2-difluoroethyl 3,3-difluoropropanoate | >25 wt. %-95 wt. % |
| Fluorinated Ester 1 | 2,2-difluoroethyl 2,2-difluoroacetate | 0.1 wt. %-5 wt. % |
| Fluorinated Ester 1 | 2,2-difluoroethyl 2,2-difluoroacetate | >5 wt. %-25 wt. % |
| Fluorinated Ester 1 | 2,2-difluoroethyl 2,2-difluoroacetate | >25 wt. %-95 wt. % |
| Fluorinated Ester 1 | succinonitrile (SN) | 0.1 wt. %-5 wt. % |
| Fluorinated Ester 1 | succinonitrile (SN) | >5 wt. %-25 wt. % |
| Fluorinated Ester 1 | succinonitrile (SN) | >25 wt. %-95 wt. % |
| Fluorinated Ester 1 | propionitrile (PN) | 0.1 wt. %-5 wt. % |
| Fluorinated Ester 1 | propionitrile (PN) | >5 wt. %-25 wt. % |
| Fluorinated Ester 1 | propionitrile (PN) | >25 wt. %-95 wt. % |
| Fluorinated Ester 1 | butyronitrile (BN) | 0.1 wt. %-5 wt. % |
| Fluorinated Ester 1 | butyronitrile (BN) | >5 wt. %-25 wt. % |
| Fluorinated Ester 1 | butyronitrile (BN) | >25 wt. %-95 wt. % |
| Fluorinated Ester 1 | adiponitrile (ADN) | 0.1 wt. %-5 wt. % |
| Fluorinated Ester 1 | adiponitrile (ADN) | >5 wt. %-25 wt. % |
| Fluorinated Ester 1 | adiponitrile (ADN) | >25 wt. %-95 wt. % |
| Fluorinated Ester 1 | 1,3,6-hexanetricarbonitrile (HTCN) | 0.1 wt. %-5 wt. % |
| Fluorinated Ester 1 | 1,3,6-hexanetricarbonitrile (HTCN) | >5 wt. %-25 wt. % |
| Fluorinated Ester 1 | 1,3,6-hexanetricarbonitrile (HTCN) | >25 wt. %-95 wt. % |
| Fluorinated Ester 1 | trimethyl borate (TMB) | 0.1 wt. %-5 wt. % |
| Fluorinated Ester 1 | trimethyl borate (TMB) | >5 wt. %-25 wt. % |
| Fluorinated Ester 1 | trimethyl borate (TMB) | >25 wt. %-95 wt. % |
| Fluorinated Ester 1 | triphenyl borate (TPB) | 0.1 wt. %-5 wt. % |
| Fluorinated Ester 1 | triphenyl borate (TPB) | >5 wt. %-25 wt. % |
| Fluorinated Ester 1 | triphenyl borate (TPB) | >25 wt. %-95 wt. % |
| Fluorinated Ester 1 | triethyl borate (TEB) | 0.1 wt. %-5 wt. % |
| Fluorinated Ester 1 | triethyl borate (TEB) | >5 wt. %-25 wt. % |
| Fluorinated Ester 1 | triethyl borate (TEB) | >25 wt. %-95 wt. % |
| Fluorinated Ester 1 | tris(pentafluorophenyl)borane (TPFPB) | 0.1 wt. %-5 wt. % |
| Fluorinated Ester 1 | tris(pentafluorophenyl)borane (TPFPB) | >5 wt. %-25 wt. % |
| Fluorinated Ester 1 | tris(pentafluorophenyl)borane (TPFPB) | >25 wt. %-95 wt. % |
| Fluorinated Ester 1 | tris(trimethylsilyl)phosphate (TTSB) | 0.1 wt. %-5 wt. % |
| Fluorinated Ester 1 | tris(trimethylsilyl)phosphate (TTSB) | >5 wt. %-25 wt. % |
| Fluorinated Ester 1 | tris(trimethylsilyl)phosphate (TTSB) | >25 wt. %-95 wt. % |
| Fluorinated Ester 1 | tris(2,2,2-trifluoroethyl) borate (TTFEB) | 0.1 wt. %-5 wt. % |
| Fluorinated Ester 1 | tris(2,2,2-trifluoroethyl) borate (TTFEB) | >5 wt. %-25 wt. % |
| Fluorinated Ester 1 | tris(2,2,2-trifluoroethyl) borate (TTFEB) | >25 wt. %-95 wt. % |
| Fluorinated Ester 1 | trimethyl phosphate (TMP) | 0.1 wt. %-5 wt. % |
| Fluorinated Ester 1 | trimethyl phosphate (TMP) | >5 wt. %-25 wt. % |
| Fluorinated Ester 1 | trimethyl phosphate (TMP) | >25 wt. %-95 wt. % |
| Fluorinated Ester 1 | triethyl phosphate (TEP) | 0.1 wt. %-5 wt. % |
| Fluorinated Ester 1 | triethyl phosphate (TEP) | >5 wt. %-25 wt. % |
| Fluorinated Ester 1 | triethyl phosphate (TEP) | >25 wt. %-95 wt. % |
| Fluorinated Ester 1 | tris(trimethylsilyl)phosphate (TTSP) | 0.1 wt. %-5 wt. % |
| Fluorinated Ester 1 | tris(trimethylsilyl)phosphate (TTSP) | >5 wt. %-25 wt. % |
| Fluorinated Ester 1 | tris(trimethylsilyl)phosphate (TTSP) | >25 wt. %-95 wt. % |
| Fluorinated Ester 1 | tris(trimethylsilyl)phosphite (TTSPi) | 0.1 wt. %-5 wt. % |
| Fluorinated Ester 1 | tris(trimethylsilyl)phosphite (TTSPi) | >5 wt. %-25 wt. % |
| Fluorinated Ester 1 | tris(trimethylsilyl)phosphite (TTSPi) | >25 wt. %-95 wt. % |
| Fluorinated Ester 1 | tris(2,2,2-trifluoroethyl) phosphate | 0.1 wt. %-5 wt. % |
| | (TFEPa) | |
| Fluorinated Ester 1 | tris(2,2,2-trifluoroethyl) phosphate | >5 wt. %-25 wt. % |
| | (TFEPa) | |
| Fluorinated Ester 1 | tris(2,2,2-trifluoroethyl) phosphate | >25 wt. %-95 wt. % |
| | (TFEPa) | |
| Fluorinated Ester 1 | tris(2,2,2-trifluoroethyl) phosphite | 0.1 wt. %-5 wt. % |
| | (TFEPi) | |
| Fluorinated Ester 1 | tris(2,2,2-trifluoroethyl) phosphite | >5 wt. %-25 wt. % |
| | (TFEPi) | |
| Fluorinated Ester 1 | tris(2,2,2-trifluoroethyl) phosphite | >25 wt. %-95 wt. % |
| | (TFEPi) | |
| Fluorinated Ester 1 | (pentafluorophenyl)diphenyl phosphine | 0.1 wt. %-5 wt. % |
| | (PFPDPP) | |
| Fluorinated Ester 1 | (pentafluorophenyl)diphenyl phosphine | >5 wt. %-25 wt. % |
| | (PFPDPP) | |
| Fluorinated Ester 1 | (pentafluorophenyl)diphenyl phosphine | >25 wt. %-95 wt. % |
| | (PFPDPP) | |
| Fluorinated Ester 1 | tris(pentafluorophenyl) phosphine | 0.1 wt. %-5 wt. % |
| | (TPFPP) | |
| Fluorinated Ester 1 | tris(pentafluorophenyl) phosphine | >5 wt. %-25 wt. % |
| | (TPFPP) | |
| Fluorinated Ester 1 | tris(pentafluorophenyl) phosphine | >25 wt. %-95 wt. % |
| | (TPFPP) | |
| Fluorinated Ester 1 | 1,3,2-dioxathiolane-2,2-dioxide (DTD) | 0.1 wt. %-5 wt. % |
| Fluorinated Ester 1 | 1,3,2-dioxathiolane-2,2-dioxide (DTD) | >5 wt. %-25 wt. % |
| Fluorinated Ester 1 | 1,3,2-dioxathiolane-2,2-dioxide (DTD) | >25 wt. %-95 wt. % |
| Fluorinated Ester 1 | 1,3-propanesultone (PS) | 0.1 wt. %-5 wt. % |
| Fluorinated Ester 1 | 1,3-propanesultone (PS) | >5 wt. %-25 wt. % |
| Fluorinated Ester 1 | 1,3-propanesultone (PS) | >25 wt. %-95 wt. % |
| Fluorinated Ester 1 | prop-1-ene-1,3-sultone (PES) | 0.1 wt. %-5 wt. % |
| Fluorinated Ester 1 | prop-1-ene-1,3-sultone (PES) | >5 wt. %-25 wt. % |
| Fluorinated Ester 1 | prop-1-ene-1,3-sultone (PES) | >25 wt. %-95 wt. % |
| Fluorinated Ester 1 | propanediol cyclic sulfate (PCS) | 0.1 wt. %-5 wt. % |
| Fluorinated Ester 1 | propanediol cyclic sulfate (PCS) | >5 wt. %-25 wt. % |
| Fluorinated Ester 1 | propanediol cyclic sulfate (PCS) | >25 wt. %-95 wt. % |
| Fluorinated Ester 1 | 1,3,2-dioxathiolane 2-oxide | 0.1 wt. %-5 wt. % |
| Fluorinated Ester 1 | 1,3,2-dioxathiolane 2-oxide | >5 wt. %-25 wt. % |
| Fluorinated Ester 1 | 1,3,2-dioxathiolane 2-oxide | >25 wt. %-95 wt. % |
| Fluorinated Ester 1 | bis(2,2,2-trifluoroethyl) ether (BTFE) | 0.1 wt. %-5 wt. % |
| Fluorinated Ester 1 | bis(2,2,2-trifluoroethyl) ether (BTFE) | >5 wt. %-25 wt. % |
| Fluorinated Ester 1 | bis(2,2,2-trifluoroethyl) ether (BTFE) | >25 wt. %-95 wt. % |
| Fluorinated Ester 1 | 1,1,2,2-tetrafluoroethyl-2,2,3,3- | 0.1 wt. %-5 wt. % |
| | tetrafluoropropyl ether (TTE) | |
| Fluorinated Ester 1 | 1,1,2,2-tetrafluoroethyl-2,2,3,3- | >5 wt. %-25 wt. % |
| | tetrafluoropropyl ether (TTE) | |
| Fluorinated Ester 1 | 1,1,2,2-tetrafluoroethyl-2,2,3,3- | >25 wt. %-95 wt. % |
| | tetrafluoropropyl ether (TTE) | |
| Fluorinated Ester 1 | 1H,1H,5H-octafluoropentyl-1,1,2,2- | 0.1 wt. %-5 wt. % |
| | tetrafluoroethylether (OTE) | |
| Fluorinated Ester 1 | 1H,1H,5H-octafluoropentyl-1,1,2,2- | >5 wt. %-25 wt. % |
| | tetrafluoroethylether (OTE) | |
| Fluorinated Ester 1 | 1H,1H,5H-octafluoropentyl-1,1,2,2- | >25 wt. %-95 wt. % |
| | tetrafluoroethylether (OTE) | |
| Fluorinated Ester 1 | 2,2,2-trifluoroethyl 1,1,2,2- | 0.1 wt. %-5 wt. % |
| | tetrafluoroethyl ether (HFE) | |
| Fluorinated Ester 1 | 2,2,2-trifluoroethyl 1,1,2,2- | >5 wt. %-25 wt. % |
| | tetrafluoroethyl ether (HFE) | |
| Fluorinated Ester 1 | 2,2,2-trifluoroethyl 1,1,2,2- | >25 wt. %-95 wt. % |
| | tetrafluoroethyl ether (HFE) | |
| Fluorinated Ester 1 | tetrahydrofuran (THF) | 0.1 wt. %-5 wt. % |
| Fluorinated Ester 1 | tetrahydrofuran (THF) | >5 wt. %-25 wt. % |
| Fluorinated Ester 1 | tetrahydrofuran (THF) | >25 wt. %-95 wt. % |
| Fluorinated Ester 1 | 1,3-dioxolane (DOL) | 0.1 wt. %-5 wt. % |
| Fluorinated Ester 1 | 1,3-dioxolane (DOL) | >5 wt. %-25 wt. % |
| Fluorinated Ester 1 | 1,3-dioxolane (DOL) | >25 wt. %-95 wt. % |
| Fluorinated Ester 1 | 1,2-dimethoxyethane (DME) | 0.1 wt. %-5 wt. % |
| Fluorinated Ester 1 | 1,2-dimethoxyethane (DME) | >5 wt. %-25 wt. % |
| Fluorinated Ester 1 | 1,2-dimethoxyethane (DME) | >25 wt. %-95 wt. % |
| Fluorinated Ester 1 | diethylene glycol dimethyl ether | 0.1 wt. %-5 wt. % |
| | (DEGDME) | |
| Fluorinated Ester 1 | diethylene glycol dimethyl ether | >5 wt. %-25 wt. % |
| | (DEGDME) | |
| Fluorinated Ester 1 | diethylene glycol dimethyl ether | >25 wt. %-95 wt. % |
| | (DEGDME) | |
| Fluorinated Ester 1 | tetraethylene glycol dimethyl ether | 0.1 wt. %-5 wt. % |
| | (TEGDME) | |
| Fluorinated Ester 1 | tetraethylene glycol dimethyl ether | >5 wt. %-25 wt. % |
| | (TEGDME) | |
| Fluorinated Ester 1 | tetraethylene glycol dimethyl ether | >25 wt. %-95 wt. % |
| | (TEGDME) | |
| Fluorinated Ester 1 | succinic anhydride (SA) | 0.1 wt. %-5 wt. % |
| Fluorinated Ester 1 | succinic anhydride (SA) | >5 wt. %-25 wt. % |
| Fluorinated Ester 1 | succinic anhydride (SA) | >25 wt. %-95 wt. % |
| Fluorinated Ester 1 | butyric anhydride (BA) | 0.1 wt. %-5 wt. % |
| Fluorinated Ester 1 | butyric anhydride (BA) | >5 wt. %-25 wt. % |
| Fluorinated Ester 1 | butyric anhydride (BA) | >25 wt. %-95 wt. % |
| Fluorinated Ester 1 | 1,4-dioxane (DOX) | 0.1 wt. %-5 wt. % |
| Fluorinated Ester 1 | 1,4-dioxane (DOX) | >5 wt. %-25 wt. % |
| Fluorinated Ester 1 | 1,4-dioxane (DOX) | >25 wt. %-95 wt. % |
| Fluorinated Ester 1 | tetravinyl silane (TVSI) | 0.1 wt. %-5 wt. % |
| Fluorinated Ester 1 | tetravinyl silane (TVSI) | >5 wt. %-25 wt. % |
| Fluorinated Ester 1 | tetravinyl silane (TVSI) | >25 wt. %-95 wt. % |
| Fluorinated Ester 1 | 1,4-butane sultone (BS) | 0.1 wt. %-5 wt. % |
| Fluorinated Ester 1 | 1,4-butane sultone (BS) | >5 wt. %-25 wt. % |
| Fluorinated Ester 1 | 1,4-butane sultone (BS) | >25 wt. %-95 wt. % |
| Fluorinated Ester 1 | dimethyl sulfoxide (DMSO) | 0.1 wt. %-5 wt. % |
| Fluorinated Ester 1 | dimethyl sulfoxide (DMSO) | >5 wt. %-25 wt. % |
| Fluorinated Ester 1 | dimethyl sulfoxide (DMSO) | >25 wt. %-95 wt. % |
| Fluorinated Ester 1 | methylene methanedisulfonate (MMDS) | 0.1 wt. %-5 wt. % |
| Fluorinated Ester 1 | methylene methanedisulfonate (MMDS) | >5 wt. %-25 wt. % |
| Fluorinated Ester 1 | methylene methanedisulfonate (MMDS) | >25 wt. %-95 wt. % |
| Fluorinated Ester 1 | N,N-dimethylformamide (DMF) | 0.1 wt. %-5 wt. % |
| Fluorinated Ester 1 | N,N-dimethylformamide (DMF) | >5 wt. %-25 wt. % |
| Fluorinated Ester 1 | N,N-dimethylformamide (DMF) | >25 wt. %-95 wt. % |
| Fluorinated Ester 1 | gamma-butyrolactone (BL) | 0.1 wt. %-5 wt. % |
| Fluorinated Ester 1 | gamma-butyrolactone (BL) | >5 wt. %-25 wt. % |
| Fluorinated Ester 1 | gamma-butyrolactone (BL) | >25 wt. %-95 wt. % |

TABLE 7B

| | | Concentration of |
|---|---|---|
| Fluorinated Ester | Solvent | Fluorinated Ester 2 |
| Fluorinated Ester 2 | fluoroethylene carbonate (FEC) | 0.1 wt. %-5 wt. % |
| Fluorinated Ester 2 | fluoroethylene carbonate (FEC) | >5 wt. %-25 wt. % |
| Fluorinated Ester 2 | fluoroethylene carbonate (FEC) | >25 wt. %-95 wt. % |
| Fluorinated Ester 2 | propylene carbonate (PC) | 0.1 wt. %-5 wt. % |
| Fluorinated Ester 2 | propylene carbonate (PC) | >5 wt. %-25 wt. % |
| Fluorinated Ester 2 | propylene carbonate (PC) | >25 wt. %-95 wt. % |
| Fluorinated Ester 2 | dimethyl carbonate (DMC) | 0.1 wt. %-5 wt. % |
| Fluorinated Ester 2 | dimethyl carbonate (DMC) | >5 wt. %-25 wt. % |
| Fluorinated Ester 2 | dimethyl carbonate (DMC) | >25 wt. %-95 wt. % |
| Fluorinated Ester 2 | diethyl carbonate (DEC) | 0.1 wt. %-5 wt. % |
| Fluorinated Ester 2 | diethyl carbonate (DEC) | >5 wt. %-25 wt. % |
| Fluorinated Ester 2 | diethyl carbonate (DEC) | >25 wt. %-95 wt. % |
| Fluorinated Ester 2 | ethyl methyl carbonate (EMC) | 0.1 wt. %-5 wt. % |
| Fluorinated Ester 2 | ethyl methyl carbonate (EMC) | >5 wt. %-25 wt. % |
| Fluorinated Ester 2 | ethyl methyl carbonate (EMC) | >25 wt. %-95 wt. % |
| Fluorinated Ester 2 | vinyl carbonate (VC) | 0.1 wt. %-5 wt. % |
| Fluorinated Ester 2 | vinyl carbonate (VC) | >5 wt. %-25 wt. % |
| Fluorinated Ester 2 | vinyl carbonate (VC) | >25 wt. %-95 wt. % |
| Fluorinated Ester 2 | vinyl ethylene carbonate (VEC) | 0.1 wt. %-5 wt. % |
| Fluorinated Ester 2 | vinyl ethylene carbonate (VEC) | >5 wt. %-25 wt. % |
| Fluorinated Ester 2 | vinyl ethylene carbonate (VEC) | >25 wt. %-95 wt. % |
| Fluorinated Ester 2 | ethylene carbonate (EC) | 0.1 wt. %-5 wt. % |
| Fluorinated Ester 2 | ethylene carbonate (EC) | >5 wt. %-25 wt. % |
| Fluorinated Ester 2 | ethylene carbonate (EC) | >25 wt. %-95 wt. % |
| Fluorinated Ester 2 | difluoroethylene carbonate (DFEC) | 0.1 wt. %-5 wt. % |
| Fluorinated Ester 2 | difluoroethylene carbonate (DFEC) | >5 wt. %-25 wt. % |
| Fluorinated Ester 2 | difluoroethylene carbonate (DFEC) | >25 wt. %-95 wt. % |
| Fluorinated Ester 2 | 3,3,3-trifluoropropylene carbonate | 0.1 wt. %-5 wt. % |
| | (TFPC) | |
| Fluorinated Ester 2 | 3,3,3-trifluoropropylene carbonate | >5 wt. %-25 wt. % |
| | (TFPC) | |
| Fluorinated Ester 2 | 3,3,3-trifluoropropylene carbonate | >25 wt. %-95 wt. % |
| | (TFPC) | |
| Fluorinated Ester 2 | monofluoroethyl methyl carbonate | 0.1 wt. %-5 wt. % |
| | (F1EMC) | |
| Fluorinated Ester 2 | monofluoroethyl methyl carbonate | >5 wt. %-25 wt. % |
| | (F1EMC) | |
| Fluorinated Ester 2 | monofluoroethyl methyl carbonate | >25 wt. %-95 wt. % |
| | (F1EMC) | |
| Fluorinated Ester 2 | difluoroethyl methyl carbonate (F2EMC) | 0.1 wt. %-5 wt. % |
| Fluorinated Ester 2 | difluoroethyl methyl carbonate (F2EMC) | >5 wt. %-25 wt. % |
| Fluorinated Ester 2 | difluoroethyl methyl carbonate (F2EMC) | >25 wt. %-95 wt. % |
| Fluorinated Ester 2 | trifluoroethyl methyl carbonate (F3EMC) | 0.1 wt. %-5 wt. % |
| Fluorinated Ester 2 | trifluoroethyl methyl carbonate (F3EMC) | >5 wt. %-25 wt. % |
| Fluorinated Ester 2 | trifluoroethyl methyl carbonate (F3EMC) | >25 wt. %-95 wt. % |
| Fluorinated Ester 2 | bis(2,2,2-trifluoroethyl) carbonate | 0.1 wt. %-5 wt. % |
| | (TFEC) | |
| Fluorinated Ester 2 | bis(2,2,2-trifluoroethyl) carbonate | >5 wt. %-25 wt. % |
| | (TFEC) | |
| Fluorinated Ester 2 | bis(2,2,2-trifluoroethyl) carbonate | >25 wt. %-95 wt. % |
| | (TFEC) | |
| Fluorinated Ester 2 | 2,2-difluoroethyl ethyl carbonate | 0.1 wt. %-5 wt. % |
| | (DFDEC) | |
| Fluorinated Ester 2 | 2,2-difluoroethyl ethyl carbonate | >5 wt. %-25 wt. % |
| | (DFDEC) | |
| Fluorinated Ester 2 | 2,2-difluoroethyl ethyl carbonate | >25 wt. %-95 wt. % |
| | (DFDEC) | |
| Fluorinated Ester 2 | acetonitrile (AN) | 0.1 wt. %-5 wt. % |
| Fluorinated Ester 2 | acetonitrile (AN) | >5 wt. %-25 wt. % |
| Fluorinated Ester 2 | acetonitrile (AN) | >25 wt. %-95 wt. % |
| Fluorinated Ester 2 | fluoroacetonitrile | 0.1 wt. %-5 wt. % |
| Fluorinated Ester 2 | fluoroacetonitrile | >5 wt. %-25 wt. % |
| Fluorinated Ester 2 | fluoroacetonitrile | >25 wt. %-95 wt. % |
| Fluorinated Ester 2 | ethyl acetate (EA) | 0.1 wt. %-5 wt. % |
| Fluorinated Ester 2 | ethyl acetate (EA) | >5 wt. %-25 wt. % |
| Fluorinated Ester 2 | ethyl acetate (EA) | >25 wt. %-95 wt. % |
| Fluorinated Ester 2 | methyl acetate (MA) | 0.1 wt. %-5 wt. % |
| Fluorinated Ester 2 | methyl acetate (MA) | >5 wt. %-25 wt. % |
| Fluorinated Ester 2 | methyl acetate (MA) | >25 wt. %-95 wt. % |
| Fluorinated Ester 2 | methyl propanoate (MP) | 0.1 wt. %-5 wt. % |
| Fluorinated Ester 2 | methyl propanoate (MP) | >5 wt. %-25 wt. % |
| Fluorinated Ester 2 | methyl propanoate (MP) | >25 wt. %-95 wt. % |
| Fluorinated Ester 2 | methyl 3,3,3-trifluoropropanoate | 0.1 wt. %-5 wt. % |
| Fluorinated Ester 2 | methyl 3,3,3-trifluoropropanoate | >5 wt. %-25 wt. % |
| Fluorinated Ester 2 | methyl 3,3,3-trifluoropropanoate | >25 wt. %-95 wt. % |
| Fluorinated Ester 2 | ethyl 3,3,3-trifluoropropanoate | 0.1 wt. %-5 wt. % |
| Fluorinated Ester 2 | ethyl 3,3,3-trifluoropropanoate | >5 wt. %-25 wt. % |
| Fluorinated Ester 2 | ethyl 3,3,3-trifluoropropanoate | >25 wt. %-95 wt. % |
| Fluorinated Ester 2 | ethyl 2,2-difluoropropanoate | 0.1 wt. %-5 wt. % |
| Fluorinated Ester 2 | ethyl 2,2-difluoropropanoate | >5 wt. %-25 wt. % |
| Fluorinated Ester 2 | ethyl 2,2-difluoropropanoate | >25 wt. %-95 wt. % |
| Fluorinated Ester 2 | isopropyl trifluoroacetate | 0.1 wt. %-5 wt. % |
| Fluorinated Ester 2 | isopropyl trifluoroacetate | >5 wt. %-25 wt. % |
| Fluorinated Ester 2 | isopropyl trifluoroacetate | >25 wt. %-95 wt. % |
| Fluorinated Ester 2 | methyl 4,4,4-trifluorobutanoate | 0.1 wt. %-5 wt. % |
| Fluorinated Ester 2 | methyl 4,4,4-trifluorobutanoate | >5 wt. %-25 wt. % |
| Fluorinated Ester 2 | methyl 4,4,4-trifluorobutanoate | >25 wt. %-95 wt. % |
| Fluorinated Ester 2 | methyl difluoroacetate | 0.1 wt. %-5 wt. % |
| Fluorinated Ester 2 | methyl difluoroacetate | >5 wt. %-25 wt. % |
| Fluorinated Ester 2 | methyl difluoroacetate | >25 wt. %-95 wt. % |
| Fluorinated Ester 2 | 2,2,2-trifluoroethyl butanoate | 0.1 wt. %-5 wt. % |
| Fluorinated Ester 2 | 2,2,2-trifluoroethyl butanoate | >5 wt. %-25 wt. % |
| Fluorinated Ester 2 | 2,2,2-trifluoroethyl butanoate | >25 wt. %-95 wt. % |
| Fluorinated Ester 2 | methyl 2,2-difluoropropanoate | 0.1 wt. %-5 wt. % |
| Fluorinated Ester 2 | methyl 2,2-difluoropropanoate | >5 wt. %-25 wt. % |
| Fluorinated Ester 2 | methyl 2,2-difluoropropanoate | >25 wt. %-95 wt. % |
| Fluorinated Ester 2 | ethyl trifluoroacetate | 0.1 wt. %-5 wt. % |
| Fluorinated Ester 2 | ethyl trifluoroacetate | >5 wt. %-25 wt. % |
| Fluorinated Ester 2 | ethyl trifluoroacetate | >25 wt. %-95 wt. % |
| Fluorinated Ester 2 | 2,2-difluoroethyl trifluoroacetate | 0.1 wt. %-5 wt. % |
| Fluorinated Ester 2 | 2,2-difluoroethyl trifluoroacetate | >5 wt. %-25 wt. % |
| Fluorinated Ester 2 | 2,2-difluoroethyl trifluoroacetate | >25 wt. %-95 wt. % |
| Fluorinated Ester 2 | 2,2,2-trifluoroethyl acetate | 0.1 wt. %-5 wt. % |
| Fluorinated Ester 2 | 2,2,2-trifluoroethyl acetate | >5 wt. %-25 wt. % |
| Fluorinated Ester 2 | 2,2,2-trifluoroethyl acetate | >25 wt. %-95 wt. % |
| Fluorinated Ester 2 | ethyl 2,2-difluoroacetate | 0.1 wt. %-5 wt. % |
| Fluorinated Ester 2 | ethyl 2,2-difluoroacetate | >5 wt. %-25 wt. % |
| Fluorinated Ester 2 | ethyl 2,2-difluoroacetate | >25 wt. %-95 wt. % |
| Fluorinated Ester 2 | 2,2-difluoroethyl acetate | 0.1 wt. %-5 wt. % |
| Fluorinated Ester 2 | 2,2-difluoroethyl acetate | >5 wt. %-25 wt. % |
| Fluorinated Ester 2 | 2,2-difluoroethyl acetate | >25 wt. %-95 wt. % |
| Fluorinated Ester 2 | 2,2-difluoroethyl 3,3-difluoropropanoate | 0.1 wt. %-5 wt. % |
| Fluorinated Ester 2 | 2,2-difluoroethyl 3,3-difluoropropanoate | >5 wt. %-25 wt. % |
| Fluorinated Ester 2 | 2,2-difluoroethyl 3,3-difluoropropanoate | >25 wt. %-95 wt. % |
| Fluorinated Ester 2 | 2,2-difluoroethyl 2,2-difluoroacetate | 0.1 wt. %-5 wt. % |
| Fluorinated Ester 2 | 2,2-difluoroethyl 2,2-difluoroacetate | >5 wt. %-25 wt. % |
| Fluorinated Ester 2 | 2,2-difluoroethyl 2,2-difluoroacetate | >25 wt. %-95 wt. % |
| Fluorinated Ester 2 | succinonitrile (SN) | 0.1 wt. %-5 wt. % |
| Fluorinated Ester 2 | succinonitrile (SN) | >5 wt. %-25 wt. % |
| Fluorinated Ester 2 | succinonitrile (SN) | >25 wt. %-95 wt. % |
| Fluorinated Ester 2 | propionitrile (PN) | 0.1 wt. %-5 wt. % |
| Fluorinated Ester 2 | propionitrile (PN) | >5 wt. %-25 wt. % |
| Fluorinated Ester 2 | propionitrile (PN) | >25 wt. %-95 wt. % |
| Fluorinated Ester 2 | butyronitrile (BN) | 0.1 wt. %-5 wt. % |
| Fluorinated Ester 2 | butyronitrile (BN) | >5 wt. %-25 wt. % |
| Fluorinated Ester 2 | butyronitrile (BN) | >25 wt. %-95 wt. % |
| Fluorinated Ester 2 | adiponitrile (ADN) | 0.1 wt. %-5 wt. % |
| Fluorinated Ester 2 | adiponitrile (ADN) | >5 wt. %-25 wt. % |
| Fluorinated Ester 2 | adiponitrile (ADN) | >25 wt. %-95 wt. % |
| Fluorinated Ester 2 | 1,3,6-hexanetricarbonitrile (HTCN) | 0.1 wt. %-5 wt. % |
| Fluorinated Ester 2 | 1,3,6-hexanetricarbonitrile (HTCN) | >5 wt. %-25 wt. % |
| Fluorinated Ester 2 | 1,3,6-hexanetricarbonitrile (HTCN) | >25 wt. %-95 wt. % |
| Fluorinated Ester 2 | trimethyl borate (TMB) | 0.1 wt. %-5 wt. % |
| Fluorinated Ester 2 | trimethyl borate (TMB) | >5 wt. %-25 wt. % |
| Fluorinated Ester 2 | trimethyl borate (TMB) | >25 wt. %-95 wt. % |
| Fluorinated Ester 2 | triphenyl borate (TPB) | 0.1 wt. %-5 wt. % |
| Fluorinated Ester 2 | triphenyl borate (TPB) | >5 wt. %-25 wt. % |
| Fluorinated Ester 2 | triphenyl borate (TPB) | >25 wt. %-95 wt. % |
| Fluorinated Ester 2 | triethyl borate (TEB) | 0.1 wt. %-5 wt. % |
| Fluorinated Ester 2 | triethyl borate (TEB) | >5 wt. %-25 wt. % |
| Fluorinated Ester 2 | triethyl borate (TEB) | >25 wt. %-95 wt. % |
| Fluorinated Ester 2 | tris(pentafluorophenyl)borane (TPFPB) | 0.1 wt. %-5 wt. % |
| Fluorinated Ester 2 | tris(pentafluorophenyl)borane (TPFPB) | >5 wt. %-25 wt. % |
| Fluorinated Ester 2 | tris(pentafluorophenyl)borane (TPFPB) | >25 wt. %-95 wt. % |
| Fluorinated Ester 2 | tris(trimethylsilyl)phosphate (TTSB) | 0.1 wt. %-5 wt. % |
| Fluorinated Ester 2 | tris(trimethylsilyl)phosphate (TTSB) | >5 wt. %-25 wt. % |
| Fluorinated Ester 2 | tris(trimethylsilyl)phosphate (TTSB) | >25 wt. %-95 wt. % |
| Fluorinated Ester 2 | tris(2,2,2-trifluoroethyl) borate (TTFEB) | 0.1 wt. %-5 wt. % |
| Fluorinated Ester 2 | tris(2,2,2-trifluoroethyl) borate (TTFEB) | >5 wt. %-25 wt. % |
| Fluorinated Ester 2 | tris(2,2,2-trifluoroethyl) borate (TTFEB) | >25 wt. %-95 wt. % |
| Fluorinated Ester 2 | trimethyl phosphate (TMP) | 0.1 wt. %-5 wt. % |
| Fluorinated Ester 2 | trimethyl phosphate (TMP) | >5 wt. %-25 wt. % |
| Fluorinated Ester 2 | trimethyl phosphate (TMP) | >25 wt. %-95 wt. % |
| Fluorinated Ester 2 | triethyl phosphate (TEP) | 0.1 wt. %-5 wt. % |
| Fluorinated Ester 2 | triethyl phosphate (TEP) | >5 wt. %-25 wt. % |
| Fluorinated Ester 2 | triethyl phosphate (TEP) | >25 wt. %-95 wt. % |
| Fluorinated Ester 2 | tris(trimethylsilyl)phosphate (TTSP) | 0.1 wt. %-5 wt. % |
| Fluorinated Ester 2 | tris(trimethylsilyl)phosphate (TTSP) | >5 wt. %-25 wt. % |
| Fluorinated Ester 2 | tris(trimethylsilyl)phosphate (TTSP) | >25 wt. %-95 wt. % |
| Fluorinated Ester 2 | tris(trimethylsilyl)phosphite (TTSPi) | 0.1 wt. %-5 wt. % |
| Fluorinated Ester 2 | tris(trimethylsilyl)phosphite (TTSPi) | >5 wt. %-25 wt. % |
| Fluorinated Ester 2 | tris(trimethylsilyl)phosphite (TTSPi) | >25 wt. %-95 wt. % |
| Fluorinated Ester 2 | tris(2,2,2-trifluoroethyl) phosphate | 0.1 wt. %-5 wt. % |
| | (TFEPa) | |
| Fluorinated Ester 2 | tris(2,2,2-trifluoroethyl) phosphate | >5 wt. %-25 wt. % |
| | (TFEPa) | |
| Fluorinated Ester 2 | tris(2,2,2-trifluoroethyl) phosphate | >25 wt. %-95 wt. % |
| | (TFEPa) | |
| Fluorinated Ester 2 | tris(2,2,2-trifluoroethyl) phosphite | 0.1 wt. %-5 wt. % |
| | (TFEPi) | |
| Fluorinated Ester 2 | tris(2,2,2-trifluoroethyl) phosphite | >5 wt. %-25 wt. % |
| | (TFEPi) | |
| Fluorinated Ester 2 | tris(2,2,2-trifluoroethyl) phosphite | >25 wt. %-95 wt. % |
| | (TFEPi) | |
| Fluorinated Ester 2 | (pentafluorophenyl)diphenyl phosphine | 0.1 wt. %-5 wt. % |
| | (PFPDPP) | |
| Fluorinated Ester 2 | (pentafluorophenyl)diphenyl phosphine | >5 wt. %-25 wt. % |
| | (PFPDPP) | |
| Fluorinated Ester 2 | (pentafluorophenyl)diphenyl phosphine | >25 wt. %-95 wt. % |
| | (PFPDPP) | |
| Fluorinated Ester 2 | tris(pentafluorophenyl) phosphine | 0.1 wt. %-5 wt. % |
| | (TPFPP) | |
| Fluorinated Ester 2 | tris(pentafluorophenyl) phosphine | >5 wt. %-25 wt. % |
| | (TPFPP) | |
| Fluorinated Ester 2 | tris(pentafluorophenyl) phosphine | >25 wt. %-95 wt. % |
| | (TPFPP) | |
| Fluorinated Ester 2 | 1,3,2-dioxathiolane-2,2-dioxide (DTD) | 0.1 wt. %-5 wt. % |
| Fluorinated Ester 2 | 1,3,2-dioxathiolane-2,2-dioxide (DTD) | >5 wt. %-25 wt. % |
| Fluorinated Ester 2 | 1,3,2-dioxathiolane-2,2-dioxide (DTD) | >25 wt. %-95 wt. % |
| Fluorinated Ester 2 | 1,3-propanesultone (PS) | 0.1 wt. %-5 wt. % |
| Fluorinated Ester 2 | 1,3-propanesultone (PS) | >5 wt. %-25 wt. % |
| Fluorinated Ester 2 | 1,3-propanesultone (PS) | >25 wt. %-95 wt. % |
| Fluorinated Ester 2 | prop-1-ene-1,3-sultone (PES) | 0.1 wt. %-5 wt. % |
| Fluorinated Ester 2 | prop-1-ene-1,3-sultone (PES) | >5 wt. %-25 wt. % |
| Fluorinated Ester 2 | prop-1-ene-1,3-sultone (PES) | >25 wt. %-95 wt. % |
| Fluorinated Ester 2 | propanediol cyclic sulfate (PCS) | 0.1 wt. %-5 wt. % |
| Fluorinated Ester 2 | propanediol cyclic sulfate (PCS) | >5 wt. %-25 wt. % |
| Fluorinated Ester 2 | propanediol cyclic sulfate (PCS) | >25 wt. %-95 wt. % |
| Fluorinated Ester 2 | 1,3,2-dioxathiolane 2-oxide | 0.1 wt. %-5 wt. % |
| Fluorinated Ester 2 | 1,3,2-dioxathiolane 2-oxide | >5 wt. %-25 wt. % |
| Fluorinated Ester 2 | 1,3,2-dioxathiolane 2-oxide | >25 wt. %-95 wt. % |
| Fluorinated Ester 2 | bis(2,2,2-trifluoroethyl) ether (BTFE) | 0.1 wt. %-5 wt. % |
| Fluorinated Ester 2 | bis(2,2,2-trifluoroethyl) ether (BTFE) | >5 wt. %-25 wt. % |
| Fluorinated Ester 2 | bis(2,2,2-trifluoroethyl) ether (BTFE) | >25 wt. %-95 wt. % |
| Fluorinated Ester 2 | 1,1,2,2-tetrafluoroethyl-2,2,3,3- | 0.1 wt. %-5 wt. % |
| | tetrafluoropropyl ether (TTE) | |
| Fluorinated Ester 2 | 1,1,2,2-tetrafluoroethyl-2,2,3,3- | >5 wt. %-25 wt. % |
| | tetrafluoropropyl ether (TTE) | |
| Fluorinated Ester 2 | 1,1,2,2-tetrafluoroethyl-2,2,3,3- | >25 wt. %-95 wt. % |
| | tetrafluoropropyl ether (TTE) | |
| Fluorinated Ester 2 | 1H,1H,5H-octafluoropentyl-1,1,2,2- | 0.1 wt. %-5 wt. % |
| | tetrafluoroethylether (OTE) | |
| Fluorinated Ester 2 | 1H,1H,5H-octafluoropentyl-1,1,2,2- | >5 wt. %-25 wt. % |
| | tetrafluoroethylether (OTE) | |
| Fluorinated Ester 2 | 1H,1H,5H-octafluoropentyl-1,1,2,2- | >25 wt. %-95 wt. % |
| | tetrafluoroethylether (OTE) | |
| Fluorinated Ester 2 | 2,2,2-trifluoroethyl 1,1,2,2- | 0.1 wt. %-5 wt. % |
| | tetrafluoroethyl ether (HFE) | |
| Fluorinated Ester 2 | 2,2,2-trifluoroethyl 1,1,2,2- | >5 wt. %-25 wt. % |
| | tetrafluoroethyl ether (HFE) | |
| Fluorinated Ester 2 | 2,2,2-trifluoroethyl 1,1,2,2- | >25 wt. %-95 wt. % |
| | tetrafluoroethyl ether (HFE) | |
| Fluorinated Ester 2 | tetrahydrofuran (THF) | 0.1 wt. %-5 wt. % |
| Fluorinated Ester 2 | tetrahydrofuran (THF) | >5 wt. %-25 wt. % |
| Fluorinated Ester 2 | tetrahydrofuran (THF) | >25 wt. %-95 wt. % |
| Fluorinated Ester 2 | 1,3-dioxolane (DOL) | 0.1 wt. %-5 wt. % |
| Fluorinated Ester 2 | 1,3-dioxolane (DOL) | >5 wt. %-25 wt. % |
| Fluorinated Ester 2 | 1,3-dioxolane (DOL) | >25 wt. %-95 wt. % |
| Fluorinated Ester 2 | 1,2-dimethoxyethane (DME) | 0.1 wt. %-5 wt. % |
| Fluorinated Ester 2 | 1,2-dimethoxyethane (DME) | >5 wt. %-25 wt. % |
| Fluorinated Ester 2 | 1,2-dimethoxyethane (DME) | >25 wt. %-95 wt. % |
| Fluorinated Ester 2 | diethylene glycol dimethyl ether | 0.1 wt. %-5 wt. % |
| | (DEGDME) | |
| Fluorinated Ester 2 | diethylene glycol dimethyl ether | >5 wt. %-25 wt. % |
| | (DEGDME) | |
| Fluorinated Ester 2 | diethylene glycol dimethyl ether | >25 wt. %-95 wt. % |
| | (DEGDME) | |
| Fluorinated Ester 2 | tetraethylene glycol dimethyl ether | 0.1 wt. %-5 wt. % |
| | (TEGDME) | |
| Fluorinated Ester 2 | tetraethylene glycol dimethyl ether | >5 wt. %-25 wt. % |
| | (TEGDME) | |
| Fluorinated Ester 2 | tetraethylene glycol dimethyl ether | >25 wt. %-95 wt. % |
| | (TEGDME) | |
| Fluorinated Ester 2 | succinic anhydride (SA) | 0.1 wt. %-5 wt. % |
| Fluorinated Ester 2 | succinic anhydride (SA) | >5 wt. %-25 wt. % |
| Fluorinated Ester 2 | succinic anhydride (SA) | >25 wt. %-95 wt. % |
| Fluorinated Ester 2 | butyric anhydride (BA) | 0.1 wt. %-5 wt. % |
| Fluorinated Ester 2 | butyric anhydride (BA) | >5 wt. %-25 wt. % |
| Fluorinated Ester 2 | butyric anhydride (BA) | >25 wt. %-95 wt. % |
| Fluorinated Ester 2 | 1,4-dioxane (DOX) | 0.1 wt. %-5 wt. % |
| Fluorinated Ester 2 | 1,4-dioxane (DOX) | >5 wt. %-25 wt. % |
| Fluorinated Ester 2 | 1,4-dioxane (DOX) | >25 wt. %-95 wt. % |
| Fluorinated Ester 2 | tetravinyl silane (TVSI) | 0.1 wt. %-5 wt. % |
| Fluorinated Ester 2 | tetravinyl silane (TVSI) | >5 wt. %-25 wt. % |
| Fluorinated Ester 2 | tetravinyl silane (TVSI) | >25 wt. %-95 wt. % |
| Fluorinated Ester 2 | 1,4-butane sultone (BS) | 0.1 wt. %-5 wt. % |
| Fluorinated Ester 2 | 1,4-butane sultone (BS) | >5 wt. %-25 wt. % |
| Fluorinated Ester 2 | 1,4-butane sultone (BS) | >25 wt. %-95 wt. % |
| Fluorinated Ester 2 | dimethyl sulfoxide (DMSO) | 0.1 wt. %-5 wt. % |
| Fluorinated Ester 2 | dimethyl sulfoxide (DMSO) | >5 wt. %-25 wt. % |
| Fluorinated Ester 2 | dimethyl sulfoxide (DMSO) | >25 wt. %-95 wt. % |
| Fluorinated Ester 2 | methylene methanedisulfonate (MMDS) | 0.1 wt. %-5 wt. % |
| Fluorinated Ester 2 | methylene methanedisulfonate (MMDS) | >5 wt. %-25 wt. % |
| Fluorinated Ester 2 | methylene methanedisulfonate (MMDS) | >25 wt. %-95 wt. % |
| Fluorinated Ester 2 | N,N-dimethylformamide (DMF) | 0.1 wt. %-5 wt. % |
| Fluorinated Ester 2 | N,N-dimethylformamide (DMF) | >5 wt. %-25 wt. % |
| Fluorinated Ester 2 | N,N-dimethylformamide (DMF) | >25 wt. %-95 wt. % |
| Fluorinated Ester 2 | gamma-butyrolactone (BL) | 0.1 wt. %-5 wt. % |
| Fluorinated Ester 2 | gamma-butyrolactone (BL) | >5 wt. %-25 wt. % |
| Fluorinated Ester 2 | gamma-butyrolactone (BL) | >25 wt. %-95 wt. % |

Tables 7C and 7D includes such solvents that are not one of Fluorinated Esters 1 and 2 that can be included in the electrolyte. The amount of supplemental solvent in the electrolyte can be from about 0.5 volume percent (vol. %) to about 99.0 vol. %, based on a total volume of the electrolyte. Fluorinated Esters 1 and 2 can be present in an amount of from 0.1 vol. % to 5 vol. % (e.g., as an additive), from greater than 5 vol. % to 25 vol. % (e.g., as a secondary solvent), or from greater than 25 vol. % to 90 vol. % (e.g., as a primary solvent), or within any range encompassed by any two of the foregoing values as endpoints, based on a total volume of the electrolyte. The amount of Fluorinated Esters 1 and 2 can be selected depending on a battery being formed and uses thereof. It is understood that the invention comprises, consists essentially of, or consists of the compositions identified in Table 7C. It is understood that the invention comprises, consists essentially of, or consists of the compositions identified in Table 7D.

TABLE 7C

| Fluorinated | | Concentration of |
|---|---|---|
| Ester | Solvent | Fluorinated Ester 1 |
| Fluorinated | fluoroethylene carbonate (FEC) | 0.1 vol. %-5 vol. % |
| Ester 1 | | |
| Fluorinated | fluoroethylene carbonate (FEC) | >5 vol. %-25 vol. % |
| Ester 1 | | |
| Fluorinated | fluoroethylene carbonate (FEC) | >25 vol. %-95 vol. % |
| Ester 1 | | |
| Fluorinated | propylene carbonate (PC) | 0.1 vol. %-5 vol. % |
| Ester 1 | | |
| Fluorinated | propylene carbonate (PC) | >5 vol. %-25 vol. % |
| Ester 1 | | |
| Fluorinated | propylene carbonate (PC) | >25 vol. %-95 vol. % |
| Ester 1 | | |
| Fluorinated | dimethyl carbonate (DMC) | 0.1 vol. %-5 vol. % |
| Ester 1 | | |
| Fluorinated | dimethyl carbonate (DMC) | >5 vol. %-25 vol. % |
| Ester 1 | | |
| Fluorinated | dimethyl carbonate (DMC) | >25 vol. %-95 vol. % |
| Ester 1 | | |
| Fluorinated | diethyl carbonate (DEC) | 0.1 vol. %-5 vol. % |
| Ester 1 | | |
| Fluorinated | diethyl carbonate (DEC) | >5 vol. %-25 vol. % |
| Ester 1 | | |
| Fluorinated | diethyl carbonate (DEC) | >25 vol. %-95 vol. % |
| Ester 1 | | |
| Fluorinated | ethyl methyl carbonate (EMC) | 0.1 vol. %-5 vol. % |
| Ester 1 | | |
| Fluorinated | ethyl methyl carbonate (EMC) | >5 vol. %-25 vol. % |
| Ester 1 | | |
| Fluorinated | ethyl methyl carbonate (EMC) | >25 vol. %-95 vol. % |
| Ester 1 | | |
| Fluorinated | vinyl carbonate (VC) | 0.1 vol. %-5 vol. % |
| Ester 1 | | |
| Fluorinated | vinyl carbonate (VC) | >5 vol. %-25 vol. % |
| Ester 1 | | |
| Fluorinated | vinyl carbonate (VC) | >25 vol. %-95 vol. % |
| Ester 1 | | |
| Fluorinated | vinyl ethylene carbonate (VEC) | 0.1 vol. %-5 vol. % |
| Ester 1 | | |
| Fluorinated | vinyl ethylene carbonate (VEC) | >5 vol. %-25 vol. % |
| Ester 1 | | |
| Fluorinated | vinyl ethylene carbonate (VEC) | >25 vol. %-95 vol. % |
| Ester 1 | | |
| Fluorinated | ethylene carbonate (EC) | 0.1 vol. %-5 vol. % |
| Ester 1 | | |
| Fluorinated | ethylene carbonate (EC) | >5 vol. %-25 vol. % |
| Ester 1 | | |
| Fluorinated | ethylene carbonate (EC) | >25 vol. %-95 vol. % |
| Ester 1 | | |
| Fluorinated | difluoroethylene carbonate (DFEC) | 0.1 vol. %-5 vol. % |
| Ester 1 | | |
| Fluorinated | difluoroethylene carbonate (DFEC) | >5 vol. %-25 vol. % |
| Ester 1 | | |
| Fluorinated | difluoroethylene carbonate (DFEC) | >25 vol. %-95 vol. % |
| Ester 1 | | |
| Fluorinated | 3,3,3-trifluoropropylene carbonate | 0.1 vol. %-5 vol. % |
| Ester 1 | (TFPC) | |
| Fluorinated | 3,3,3-trifluoropropylene carbonate | >5 vol. %-25 vol. % |
| Ester 1 | (TFPC) | |
| Fluorinated | 3,3,3-trifluoropropylene carbonate | >25 vol. %-95 vol. % |
| Ester 1 | (TFPC) | |
| Fluorinated | monofluoroethyl methyl carbonate | 0.1 vol. %-5 vol. % |
| Ester 1 | (F1EMC) | |
| Fluorinated | monofluoroethyl methyl carbonate | >5 vol. %-25 vol. % |
| Ester 1 | (F1EMC) | |
| Fluorinated | monofluoroethyl methyl carbonate | >25 vol. %-95 vol. % |
| Ester 1 | (F1EMC) | |
| Fluorinated | difluoroethyl methylcarbonate | 0.1 vol. %-5 vol. % |
| Ester 1 | (F2EMC) | |
| Fluorinated | difluoroethyl methyl carbonate | >5 vol. %-25 vol. % |
| Ester 1 | (F2EMC) | |
| Fluorinated | difluoroethyl methyl carbonate | >25 vol. %-95 vol. % |
| Ester 1 | (F2EMC) | |
| Fluorinated | trifluoroethyl methyl carbonate | 0.1 vol. %-5 vol. % |
| Ester 1 | (F3EMC) | |
| Fluorinated | trifluoroethyl methyl carbonate | >5 vol. %-25 vol. % |
| Ester 1 | (F3EMC) | |
| Fluorinated | trifluoroethyl methyl carbonate | >25 vol. %-95 vol. % |
| Ester 1 | (F3EMC) | |
| Fluorinated | bis(2,2,2-trifluoroethyl) carbonate | 0.1 vol. %-5 vol. % |
| Ester 1 | (TFEC) | |
| Fluorinated | bis(2,2,2-trifluoroethyl) carbonate | >5 vol. %-25 vol. % |
| Ester 1 | (TFEC) | |
| Fluorinated | bis(2,2,2-trifluoroethyl) carbonate | >25 vol. %-95 vol. % |
| Ester 1 | (TFEC) | |
| Fluorinated | 2,2-difluoroethyl ethyl carbonate | 0.1 vol. %-5 vol. % |
| Ester 1 | (DFDEC) | |
| Fluorinated | 2,2-difluoroethyl ethyl carbonate | >5 vol. %-25 vol. % |
| Ester 1 | (DFDEC) | |
| Fluorinated | 2,2-difluoroethyl ethyl carbonate | >25 vol. %-95 vol. % |
| Ester 1 | (DFDEC) | |
| Fluorinated | acetonitrile (AN) | 0.1 vol. %-5 vol. % |
| Ester 1 | | |
| Fluorinated | acetonitrile (AN) | >5 vol. %-25 vol. % |
| Ester 1 | | |
| Fluorinated | acetonitrile (AN) | >25 vol. %-95 vol. % |
| Ester 1 | | |
| Fluorinated | fluoroacetonitrile | 0.1 vol. %-5 vol. % |
| Ester 1 | | |
| Fluorinated | fluoroacetonitrile | >5 vol. %-25 vol. % |
| Ester 1 | | |
| Fluorinated | fluoroacetonitrile | >25 vol. %-95 vol. % |
| Ester 1 | | |
| Fluorinated | ethyl acetate (EA) | 0.1 vol. %-5 vol. % |
| Ester 1 | | |
| Fluorinated | ethyl acetate (EA) | >5 vol. %-25 vol. % |
| Ester 1 | | |
| Fluorinated | ethyl acetate (EA) | >25 vol. %-95 vol. % |
| Ester 1 | | |
| Fluorinated | methyl acetate (MA) | 0.1 vol. %-5 vol. % |
| Ester 1 | | |
| Fluorinated | methyl acetate (MA) | >5 vol. %-25 vol. % |
| Ester 1 | | |
| Fluorinated | methyl acetate (MA) | >25 vol. %-95 vol. % |
| Ester 1 | | |
| Fluorinated | methyl propanoate (MP) | 0.1 vol. %-5 vol. % |
| Ester 1 | | |
| Fluorinated | methyl propanoate (MP) | >5 vol. %-25 vol. % |
| Ester 1 | | |
| Fluorinated | methyl propanoate (MP) | >25 vol. %-95 vol. % |
| Ester 1 | | |
| Fluorinated | methyl 3,3,3-trifluoropropanoate | 0.1 vol. %-5 vol. % |
| Ester 1 | | |
| Fluorinated | methyl 3,3,3-trifluoropropanoate | >5 vol. %-25 vol. % |
| Ester 1 | | |
| Fluorinated | methyl 3,3,3-trifluoropropanoate | >25 vol. %-95 vol. % |
| Ester 1 | | |
| Fluorinated | ethyl 3,3,3-trifluoropropanoate | 0.1 vol. %-5 vol. % |
| Ester 1 | | |
| Fluorinated | ethyl 3,3,3-trifluoropropanoate | >5 vol. %-25 vol. % |
| Ester 1 | | |
| Fluorinated | ethyl 3,3,3-trifluoropropanoate | >25 vol. %-95 vol. % |
| Ester 1 | | |
| Fluorinated | ethyl 2,2-difluoropropanoate | 0.1 vol. %-5 vol. % |
| Ester 1 | | |
| Fluorinated | ethyl 2,2-difluoropropanoate | >5 vol. %-25 vol. % |
| Ester 1 | | |
| Fluorinated | ethyl 2,2-difluoropropanoate | >25 vol. %-95 vol. % |
| Ester 1 | | |
| Fluorinated | isopropyl trifluoroacetate | 0.1 vol. %-5 vol. % |
| Ester 1 | | |
| Fluorinated | isopropyl trifluoroacetate | >5 vol. %-25 vol. % |
| Ester 1 | | |
| Fluorinated | isopropyl trifluoroacetate | >25 vol. %-95 vol. % |
| Ester 1 | | |
| Fluorinated | methyl 4,4,4-trifluorobutanoate | 0.1 vol. %-5 vol. % |
| Ester 1 | | |
| Fluorinated | methyl 4,4,4-trifluorobutanoate | >5 vol. %-25 vol. % |
| Ester 1 | | |
| Fluorinated | methyl 4,4,4-trifluorobutanoate | >25 vol. %-95 vol. % |
| Ester 1 | | |
| Fluorinated | methyl difluoroacetate | 0.1 vol. %-5 vol. % |
| Ester 1 | | |
| Fluorinated | methyl difluoroacetate | >5 vol. %-25 vol. % |
| Ester 1 | | |
| Fluorinated | methyl difluoroacetate | >25 vol. %-95 vol. % |
| Ester 1 | | |
| Fluorinated | 2,2,2-trifluoroethyl butanoate | 0.1 vol. %-5 vol. % |
| Ester 1 | | |
| Fluorinated | 2,2,2-trifluoroethyl butanoate | >5 vol. %-25 vol. % |
| Ester 1 | | |
| Fluorinated | 2,2,2-trifluoroethyl butanoate | >25 vol. %-95 vol. % |
| Ester 1 | | |
| Fluorinated | methyl 2,2-difluoropropanoate | 0.1 vol. %-5 vol. % |
| Ester 1 | | |
| Fluorinated | methyl 2,2-difluoropropanoate | >5 vol. %-25 vol. % |
| Ester 1 | | |
| Fluorinated | methyl 2,2-difluoropropanoate | >25 vol. %-95 vol. % |
| Ester 1 | | |
| Fluorinated | ethyl trifluoroacetate | 0.1 vol. %-5 vol. % |
| Ester 1 | | |
| Fluorinated | ethyl trifluoroacetate | >5 vol. %-25 vol. % |
| Ester 1 | | |
| Fluorinated | ethyl trifluoroacetate | >25 vol. %-95 vol. % |
| Ester 1 | | |
| Fluorinated | 2,2-difluoroethyl trifluoroacetate | 0.1 vol. %-5 vol. % |
| Ester 1 | | |
| Fluorinated | 2,2-difluoroethyl trifluoroacetate | >5 vol. %-25 vol. % |
| Ester 1 | | |
| Fluorinated | 2,2-difluoroethyl trifluoroacetate | >25 vol. %-95 vol. % |
| Ester 1 | | |
| Fluorinated | 2,2,2-trifluoroethyl acetate | 0.1 vol. %-5 vol. % |
| Ester 1 | | |
| Fluorinated | 2,2,2-trifluoroethyl acetate | >5 vol. %-25 vol. % |
| Ester 1 | | |
| Fluorinated | 2,2,2-trifluoroethyl acetate | >25 vol. %-95 vol. % |
| Ester 1 | | |
| Fluorinated | ethyl 2,2-difluoroacetate | 0.1 vol. %-5 vol. % |
| Ester 1 | | |
| Fluorinated | ethyl 2,2-difluoroacetate | >5 vol. %-25 vol. % |
| Ester 1 | | |
| Fluorinated | ethyl 2,2-difluoroacetate | >25 vol. %-95 vol. % |
| Ester 1 | | |
| Fluorinated | 2,2-difluoroethyl acetate | 0.1 vol. %-5 vol. % |
| Ester 1 | | |
| Fluorinated | 2,2-difluoroethyl acetate | >5 vol. %-25 vol. % |
| Ester 1 | | |
| Fluorinated | 2,2-difluoroethyl acetate | >25 vol. %-95 vol. % |
| Ester 1 | | |
| Fluorinated | 2,2-difluoroethyl | 0.1 vol. %-5 vol. % |
| Ester 1 | 3,3-difluoropropanoate | |
| Fluorinated | 2,2-difluoroethyl | >5 vol. %-25 vol. % |
| Ester 1 | 3,3-difluoropropanoate | |
| Fluorinated | 2,2-difluoroethyl | >25 vol. %-95 vol. % |
| Ester 1 | 3,3-difluoropropanoate | |
| Fluorinated | 2,2-difluoroethyl | 0.1 vol. %-5 vol. % |
| Ester 1 | 2,2-difluoroacetate | |
| Fluorinated | 2,2-difluoroethyl | >5 vol. %-25 vol. % |
| Ester 1 | 2,2-difluoroacetate | |
| Fluorinated | 2,2-difluoroethyl | >25 vol. %-95 vol. % |
| Ester 1 | 2,2-difluoroacetate | |
| Fluorinated | succinonitrile (SN) | 0.1 vol. %-5 vol. % |
| Ester 1 | | |
| Fluorinated | succinonitrile (SN) | >5 vol. %-25 vol. % |
| Ester 1 | | |
| Fluorinated | succinonitrile (SN) | >25 vol. %-95 vol. % |
| Ester 1 | | |
| Fluorinated | propionitrile (PN) | 0.1 vol. %-5 vol. % |
| Ester 1 | | |
| Fluorinated | propionitrile (PN) | >5 vol. %-25 vol. % |
| Ester 1 | | |
| Fluorinated | propionitrile (PN) | >25 vol. %-95 vol. % |
| Ester 1 | | |
| Fluorinated | butyronitrile (BN) | 0.1 vol. %-5 vol. % |
| Ester 1 | | |
| Fluorinated | butyronitrile (BN) | >5 vol. %-25 vol. % |
| Ester 1 | | |
| Fluorinated | butyronitrile (BN) | >25 vol. %-95 vol. % |
| Ester 1 | | |
| Fluorinated | adiponitrile (ADN) | 0.1 vol. %-5 vol. % |
| Ester 1 | | |
| Fluorinated | adiponitrile (ADN) | >5 vol. %-25 vol. % |
| Ester 1 | | |
| Fluorinated | adiponitrile (ADN) | >25 vol. %-95 vol. % |
| Ester 1 | | |
| Fluorinated | 1,3,6-hexanetricarbonitrile (HTCN) | 0.1 vol. %-5 vol. % |
| Ester 1 | | |
| Fluorinated | 1,3,6-hexanetricarbonitrile (HTCN) | >5 vol. %-25 vol. % |
| Ester 1 | | |
| Fluorinated | 1,3,6-hexanetricarbonitrile (HTCN) | >25 vol. %-95 vol. % |
| Ester 1 | | |
| Fluorinated | trimethyl borate (TMB) | 0.1 vol. %-5 vol. % |
| Ester 1 | | |
| Fluorinated | trimethyl borate (TMB) | >5 vol. %-25 vol. % |
| Ester 1 | | |
| Fluorinated | trimethyl borate (TMB) | >25 vol. %-95 vol. % |
| Ester 1 | | |
| Fluorinated | triphenyl borate (TPB) | 0.1 vol. %-5 vol. % |
| Ester 1 | | |
| Fluorinated | triphenyl borate (TPB) | >5 vol. %-25 vol. % |
| Ester 1 | | |
| Fluorinated | triphenyl borate (TPB) | >25 vol. %-95 vol. % |
| Ester 1 | | |
| Fluorinated | triethyl borate (TEB) | 0.1 vol. %-5 vol. % |
| Ester 1 | | |
| Fluorinated | triethyl borate (TEB) | >5 vol. %-25 vol. % |
| Ester 1 | | |
| Fluorinated | triethyl borate (TEB) | >25 vol. %-95 vol. % |
| Ester 1 | | |
| Fluorinated | tris(pentafluorophenyl)borane | 0.1 vol. %-5 vol. % |
| Ester 1 | (TPFPB) | |
| Fluorinated | tris(pentafluorophenyl)borane | >5 vol. %-25 vol. % |
| Ester 1 | (TPFPB) | |
| Fluorinated | tris(pentafluorophenyl)borane | >25 vol. %-95 vol. % |
| Ester 1 | (TPFPB) | |
| Fluorinated | tris(trimethylsilyl)phosphate | 0.1 vol. %-5 vol. % |
| Ester 1 | (TTSB) | |
| Fluorinated | tris(trimethylsilyl)phosphate | >5 vol. %-25 vol. % |
| Ester 1 | (TTSB) | |
| Fluorinated | tris(trimethylsilyl)phosphate | >25 vol. %-95 vol. % |
| Ester 1 | (TTSB) | |
| Fluorinated | tris(2,2,2-trifluoroethyl) borate | 0.1 vol. %-5 vol. % |
| Ester 1 | (TTFEB) | |
| Fluorinated | tris(2,2,2-trifluoroethyl) borate | >5 vol. %-25 vol. % |
| Ester 1 | (TTFEB) | |
| Fluorinated | tris(2,2,2-trifluoroethyl) borate | >25 vol. %-95 vol. % |
| Ester 1 | (TTFEB) | |
| Fluorinated | trimethyl phosphate (TMP) | 0.1 vol. %-5 vol. % |
| Ester 1 | | |
| Fluorinated | trimethyl phosphate (TMP) | >5 vol. %-25 vol. % |
| Ester 1 | | |
| Fluorinated | trimethyl phosphate (TMP) | >25 vol. %-95 vol. % |
| Ester 1 | | |
| Fluorinated | triethyl phosphate (TEP) | 0.1 vol. %-5 vol. % |
| Ester 1 | | |
| Fluorinated | triethyl phosphate (TEP) | >5 vol. %-25 vol. % |
| Ester 1 | | |
| Fluorinated | triethyl phosphate (TEP) | >25 vol. %-95 vol. % |
| Ester 1 | | |
| Fluorinated | tris(trimethylsilyl)phosphate | 0.1 vol. %-5 vol. % |
| Ester 1 | (TTSP) | |
| Fluorinated | tris(trimethylsilyl)phosphate | >5 vol. %-25 vol. % |
| Ester 1 | (TTSP) | |
| Fluorinated | tris(trimethylsilyl)phosphate | >25 vol. %-95 vol. % |
| Ester 1 | (TTSP) | |
| Fluorinated | tris(trimethylsilyl)phosphite | 0.1 vol. %-5 vol. % |
| Ester 1 | (TTSPi) | |
| Fluorinated | tris(trimethylsilyl)phosphite | >5 vol. %-25 vol. % |
| Ester 1 | (TTSPi) | |
| Fluorinated | tris(trimethylsilyl)phosphite | >25 vol. %-95 vol. % |
| Ester 1 | (TTSPi) | |
| Fluorinated | tris(2,2,2-trifluoroethyl) | 0.1 vol. %-5 vol. % |
| Ester 1 | phosphate (TFEPa) | |
| Fluorinated | tris(2,2,2-trifluoroethyl) | >5 vol. %-25 vol. % |
| Ester 1 | phosphate (TFEPa) | |
| Fluorinated | tris(2,2,2-trifluoroethyl) | >25 vol. %-95 vol. % |
| Ester 1 | phosphate (TFEPa) | |
| Fluorinated | tris(2,2,2-trifluoroethyl) | 0.1 vol. %-5 vol. % |
| Ester 1 | phosphite (TFEPi) | |
| Fluorinated | tris(2,2,2-trifluoroethyl) | >5 vol. %-25 vol. % |
| Ester 1 | phosphite (TFEPi) | |
| Fluorinated | tris(2,2,2-trifluoroethyl) | >25 vol. %-95 vol. % |
| Ester 1 | phosphite (TFEPi) | |
| Fluorinated | (pentafluorophenyl)diphenyl | 0.1 vol. %-5 vol. % |
| Ester 1 | phosphine (PFPDPP) | |
| Fluorinated | (pentafluorophenyl)diphenyl | >5 vol. %-25 vol. % |
| Ester 1 | phosphine (PFPDPP) | |
| Fluorinated | (pentafluorophenyl)diphenyl | >25 vol. %-95 vol. % |
| Ester 1 | phosphine (PFPDPP) | |
| Fluorinated | tris(pentafluorophenyl) | 0.1 vol. %-5 vol. % |
| Ester 1 | phosphine (TPFPP) | |
| Fluorinated | tris(pentafluorophenyl) | >5 vol. %-25 vol. % |
| Ester 1 | phosphine (TPFPP) | |
| Fluorinated | tris(pentafluorophenyl) | >25 vol. %-95 vol. % |
| Ester 1 | phosphine (TPFPP) | |
| Fluorinated | 1,3,2-dioxathiolane-2,2-dioxide | 0.1 vol. %-5 vol. % |
| Ester 1 | (DTD) | |
| Fluorinated | 1,3,2-dioxathiolane-2,2-dioxide | >5 vol. %-25 vol. % |
| Ester 1 | (DTD) | |
| Fluorinated | 1,3,2-dioxathiolane-2,2-dioxide | >25 vol. %-95 vol. % |
| Ester 1 | (DTD) | |
| Fluorinated | 1,3-propanesultone (PS) | 0.1 vol. %-5 vol. % |
| Ester 1 | | |
| Fluorinated | 1,3-propanesultone (PS) | >5 vol. %-25 vol. % |
| Ester 1 | | |
| Fluorinated | 1,3-propanesultone (PS) | >25 vol. %-95 vol. % |
| Ester 1 | | |
| Fluorinated | prop-1-ene-1,3-sultone (PES) | 0.1 vol. %-5 vol. % |
| Ester 1 | | |
| Fluorinated | prop-1-ene-1,3-sultone (PES) | >5 vol. %-25 vol. % |
| Ester 1 | | |
| Fluorinated | prop-1-ene-1,3-sultone (PES) | >25 vol. %-95 vol. % |
| Ester 1 | | |
| Fluorinated | propanediol cyclic sulfate (PCS) | 0.1 vol. %-5 vol. % |
| Ester 1 | | |
| Fluorinated | propanediol cyclic sulfate (PCS) | >5 vol. %-25 vol. % |
| Ester 1 | | |
| Fluorinated | propanediol cyclic sulfate (PCS) | >25 vol. %-95 vol. % |
| Ester 1 | | |
| Fluorinated | 1,3,2-dioxathiolane 2-oxide | 0.1 vol. %-5 vol. % |
| Ester 1 | | |
| Fluorinated | 1,3,2-dioxathiolane 2-oxide | >5 vol. %-25 vol. % |
| Ester 1 | | |
| Fluorinated | 1,3,2-dioxathiolane 2-oxide | >25 vol. %-95 vol. % |
| Ester 1 | | |
| Fluorinated | bis(2,2,2-trifluoroethyl) ether | 0.1 vol. %-5 vol. % |
| Ester 1 | (BTFE) | |
| Fluorinated | bis(2,2,2-trifluoroethyl) ether | >5 vol. %-25 vol. % |
| Ester 1 | (BTFE) | |
| Fluorinated | bis(2,2,2-trifluoroethyl) ether | >25 vol. %-95 vol. % |
| Ester 1 | (BTFE) | |
| Fluorinated | 1,1,2,2-tetrafluoroethyl- | 0.1 vol. %-5 vol. % |
| Ester 1 | 2,2,3,3-tetrafluoropropyl ether | |
| | (TTE) | |
| Fluorinated | 1,1,2,2-tetrafluoroethyl- | >5 vol. %-25 vol. % |
| Ester 1 | 2,2,3,3-tetrafluoropropyl ether | |
| | (TTE) | |
| Fluorinated | 1,1,2,2-tetrafluoroethyl- | >25 vol. %-95 vol. % |
| Ester 1 | 2,2,3,3-tetrafluoropropyl ether | |
| | (TTE) | |
| Fluorinated | 1H,1H,5H-octafluoropentyl- | 0.1 vol. %-5 vol. % |
| Ester 1 | 1,1,2,2-tetrafluoroethylether | |
| | (OTE) | |
| Fluorinated | 1H,1H,5H-octafluoropentyl- | >5 vol. %-25 vol. % |
| Ester 1 | 1,1,2,2-tetrafluoroethylether | |
| | (OTE) | |
| Fluorinated | 1H,1H,5H-octafluoropentyl- | >25 vol. %-95 vol. % |
| Ester 1 | 1,1,2,2-tetrafluoroethylether | |
| | (OTE) | |
| Fluorinated | 2,2,2-trifluoroethyl | 0.1 vol. %-5 vol. % |
| Ester 1 | 1,1,2,2-tetrafluoroethyl ether | |
| | (HFE) | |
| Fluorinated | 2,2,2-trifluoroethyl | >5 vol. %-25 vol. % |
| Ester 1 | 1,1,2,2-tetrafluoroethyl ether | |
| | (HFE) | |
| Fluorinated | 2,2,2-trifluoroethyl | >25 vol. %-95 vol. % |
| Ester 1 | 1,1,2,2-tetrafluoroethyl ether | |
| | (HFE) | |
| Fluorinated | tetrahydrofuran (THF) | 0.1 vol. %-5 vol. % |
| Ester 1 | | |
| Fluorinated | tetrahydrofuran (THF) | >5 vol. %-25 vol. % |
| Ester 1 | | |
| Fluorinated | tetrahydrofuran (THF) | >25 vol. %-95 vol. % |
| Ester 1 | | |
| Fluorinated | 1,3-dioxolane (DOL) | 0.1 vol. %-5 vol. % |
| Ester 1 | | |
| Fluorinated | 1,3-dioxolane (DOL) | >5 vol. %-25 vol. % |
| Ester 1 | | |
| Fluorinated | 1,3-dioxolane (DOL) | >25 vol. %-95 vol. % |
| Ester 1 | | |
| Fluorinated | 1,2-dimethoxyethane (DME) | 0.1 vol. %-5 vol. % |
| Ester 1 | | |
| Fluorinated | 1,2-dimethoxyethane (DME) | >5 vol. %-25 vol. % |
| Ester 1 | | |
| Fluorinated | 1,2-dimethoxyethane (DME) | >25 vol. %-95 vol. % |
| Ester 1 | | |
| Fluorinated | diethylene glycol dimethyl ether | 0.1 vol. %-5 vol. % |
| Ester 1 | (DEGDME) | |
| Fluorinated | diethylene glycol dimethyl ether | >5 vol. %-25 vol. % |
| Ester 1 | (DEGDME) | |
| Fluorinated | diethylene glycol dimethyl ether | >25 vol. %-95 vol. % |
| Ester 1 | (DEGDME) | |
| Fluorinated | tetraethylene glycol dimethyl ether | 0.1 vol. %-5 vol. % |
| Ester 1 | (TEGDME) | |
| Fluorinated | tetraethylene glycol dimethyl ether | >5 vol. %-25 vol. % |
| Ester 1 | (TEGDME) | |
| Fluorinated | tetraethylene glycol dimethyl ether | >25 vol. %-95 vol. % |
| Ester 1 | (TEGDME) | |
| Fluorinated | succinic anhydride (SA) | 0.1 vol. %-5 vol. % |
| Ester 1 | | |
| Fluorinated | succinic anhydride (SA) | >5 vol. %-25 vol. % |
| Ester 1 | | |
| Fluorinated | succinic anhydride (SA) | >25 vol. %-95 vol. % |
| Ester 1 | | |
| Fluorinated | butyric anhydride (BA) | 0.1 vol. %-5 vol. % |
| Ester 1 | | |
| Fluorinated | butyric anhydride (BA) | >5 vol. %-25 vol. % |
| Ester 1 | | |
| Fluorinated | butyric anhydride (BA) | >25 vol. %-95 vol. % |
| Ester 1 | | |
| Fluorinated | 1,4-dioxane (DOX) | 0.1 vol. %-5 vol. % |
| Ester 1 | | |
| Fluorinated | 1,4-dioxane (DOX) | >5 vol. %-25 vol. % |
| Ester 1 | | |
| Fluorinated | 1,4-dioxane (DOX) | >25 vol. %-95 vol. % |
| Ester 1 | | |
| Fluorinated | tetravinyl silane (TVSI) | 0.1 vol. %-5 vol. % |
| Ester 1 | | |
| Fluorinated | tetravinyl silane (TVSI) | >5 vol. %-25 vol. % |
| Ester 1 | | |
| Fluorinated | tetravinyl silane (TVSI) | >25 vol. %-95 vol. % |
| Ester 1 | | |
| Fluorinated | 1,4-butane sultone (BS) | 0.1 vol. %-5 vol. % |
| Ester 1 | | |
| Fluorinated | 1,4-butane sultone (BS) | >5 vol. %-25 vol. % |
| Ester 1 | | |
| Fluorinated | 1,4-butane sultone (BS) | >25 vol. %-95 vol. % |
| Ester 1 | | |
| Fluorinated | dimethyl sulfoxide (DMSO) | 0.1 vol. %-5 vol. % |
| Ester 1 | | |
| Fluorinated | dimethyl sulfoxide (DMSO) | >5 vol. %-25 vol. % |
| Ester 1 | | |
| Fluorinated | dimethyl sulfoxide (DMSO) | >25 vol. %-95 vol. % |
| Ester 1 | | |
| Fluorinated | methylene methanedisulfonate | 0.1 vol. %-5 vol. % |
| Ester 1 | (MMDS) | |
| Fluorinated | methylene methanedisulfonate | >5 vol. %-25 vol. % |
| Ester 1 | (MMDS) | |
| Fluorinated | methylene methanedisulfonate | >25 vol. %-95 vol. % |
| Ester 1 | (MMDS) | |
| Fluorinated | N,N-dimethylformamide (DMF) | 0.1 vol. %-5 vol. % |
| Ester 1 | | |
| Fluorinated | N,N-dimethylformamide (DMF) | >5 vol. %-25 vol. % |
| Ester 1 | | |
| Fluorinated | N,N-dimethylformamide (DMF) | >25 vol. %-95 vol. % |
| Ester 1 | | |
| Fluorinated | gamma-butyrolactone (BL) | 0.1 vol. %-5 vol. % |
| Ester 1 | | |
| Fluorinated | gamma-butyrolactone (BL) | >5 vol. %-25 vol. % |
| Ester 1 | | |
| Fluorinated | gamma-butyrolactone (BL) | >25 vol. %-95 vol. % |
| Ester 1 | | |

TABLE 7D

| Fluorinated | | Concentration of |
|---|---|---|
| Ester | Solvent | Fluorinated Ester 2 |
| Fluorinated | fluoroethylene carbonate (FEC) | 0.1 vol. %-5 vol. % |
| Ester 2 | | |
| Fluorinated | fluoroethylene carbonate (FEC) | >5 vol. %-25 vol. % |
| Ester 2 | | |
| Fluorinated | fluoroethylene carbonate (FEC) | >25 vol. %-95 vol. % |
| Ester 2 | | |
| Fluorinated | propylene carbonate (PC) | 0.1 vol. %-5 vol. % |
| Ester 2 | | |
| Fluorinated | propylene carbonate (PC) | >5 vol. %-25 vol. % |
| Ester 2 | | |
| Fluorinated | propylene carbonate (PC) | >25 vol. %-95 vol. % |
| Ester 2 | | |
| Fluorinated | dimethyl carbonate (DMC) | 0.1 vol. %-5 vol. % |
| Ester 2 | | |
| Fluorinated | dimethyl carbonate (DMC) | >5 vol. %-25 vol. % |
| Ester 2 | | |
| Fluorinated | dimethyl carbonate (DMC) | >25 vol. %-95 vol. % |
| Ester 2 | | |
| Fluorinated | diethyl carbonate (DEC) | 0.1 vol. %-5 vol. % |
| Ester 2 | | |
| Fluorinated | diethyl carbonate (DEC) | >5 vol. %-25 vol. % |
| Ester 2 | | |
| Fluorinated | diethyl carbonate (DEC) | >25 vol. %-95 vol. % |
| Ester 2 | | |
| Fluorinated | ethyl methyl carbonate (EMC) | 0.1 vol. %-5 vol. % |
| Ester 2 | | |
| Fluorinated | ethyl methyl carbonate (EMC) | >5 vol. %-25 vol. % |
| Ester 2 | | |
| Fluorinated | ethyl methyl carbonate (EMC) | >25 vol. %-95 vol. % |
| Ester 2 | | |
| Fluorinated | vinyl carbonate (VC) | 0.1 vol. %-5 vol. % |
| Ester 2 | | |
| Fluorinated | vinyl carbonate (VC) | >5 vol. %-25 vol. % |
| Ester 2 | | |
| Fluorinated | vinyl carbonate (VC) | >25 vol. %-95 vol. % |
| Ester 2 | | |
| Fluorinated | vinyl ethylene carbonate (VEC) | 0.1 vol. %-5 vol. % |
| Ester 2 | | |
| Fluorinated | vinyl ethylene carbonate (VEC) | >5 vol. %-25 vol. % |
| Ester 2 | | |
| Fluorinated | vinyl ethylene carbonate (VEC) | >25 vol. %-95 vol. % |
| Ester 2 | | |
| Fluorinated | ethylene carbonate (EC) | 0.1 vol. %-5 vol. % |
| Ester 2 | | |
| Fluorinated | ethylene carbonate (EC) | >5 vol. %-25 vol. % |
| Ester 2 | | |
| Fluorinated | ethylene carbonate (EC) | >25 vol. %-95 vol. % |
| Ester 2 | | |
| Fluorinated | difluoroethylene carbonate (DFEC) | 0.1 vol. %-5 vol. % |
| Ester 2 | | |
| Fluorinated | difluoroethylene carbonate (DFEC) | >5 vol. %-25 vol. % |
| Ester 2 | | |
| Fluorinated | difluoroethylene carbonate (DFEC) | >25 vol. %-95 vol. % |
| Ester 2 | | |
| Fluorinated | 3,3,3-trifluoropropylene carbonate | 0.1 vol. %-5 vol. % |
| Ester 2 | (TFPC) | |
| Fluorinated | 3,3,3-trifluoropropylene carbonate | >5 vol. %-25 vol. % |
| Ester 2 | (TFPC) | |
| Fluorinated | 3,3,3-trifluoropropylene carbonate | >25 vol. %-95 vol. % |
| Ester 2 | (TFPC) | |
| Fluorinated | monofluoroethyl methyl carbonate | 0.1 vol. %-5 vol. % |
| Ester 2 | (F1EMC) | |
| Fluorinated | monofluoroethyl methyl carbonate | >5 vol. %-25 vol. % |
| Ester 2 | (F1EMC) | |
| Fluorinated | monofluoroethyl methyl carbonate | >25 vol. %-95 vol. % |
| Ester 2 | (F1EMC) | |
| Fluorinated | difluoroethyl methyl carbonate | 0.1 vol. %-5 vol. % |
| Ester 2 | (F2EMC) | |
| Fluorinated | difluoroethyl methyl carbonate | >5 vol. %-25 vol. % |
| Ester 2 | (F2EMC) | |
| Fluorinated | difluoroethyl methyl carbonate | >25 vol. %-95 vol. % |
| Ester 2 | (F2EMC) | |
| Fluorinated | trifluoroethyl methyl carbonate | 0.1 vol. %-5 vol. % |
| Ester 2 | (F3EMC) | |
| Fluorinated | trifluoroethyl methyl carbonate | >5 vol. %-25 vol. % |
| Ester 2 | (F3EMC) | |
| Fluorinated | trifluoroethyl methyl carbonate | >25 vol. %-95 vol. % |
| Ester 2 | (F3EMC) | |
| Fluorinated | bis(2,2,2-trifluoroethyl) carbonate | 0.1 vol. %-5 vol. % |
| Ester 2 | (TFEC) | |
| Fluorinated | bis(2,2,2-trifluoroethyl) carbonate | >5 vol. %-25 vol. % |
| Ester 2 | (TFEC) | |
| Fluorinated | bis(2,2,2-trifluoroethyl) carbonate | >25 vol. %-95 vol. % |
| Ester 2 | (TFEC) | |
| Fluorinated | 2,2-difluoroethyl ethyl carbonate | 0.1 vol. %-5 vol. % |
| Ester 2 | (DFDEC) | |
| Fluorinated | 2,2-difluoroethyl ethyl carbonate | >5 vol. %-25 vol. % |
| Ester 2 | (DFDEC) | |
| Fluorinated | 2,2-difluoroethyl ethyl carbonate | >25 vol. %-95 vol. % |
| Ester 2 | (DFDEC) | |
| Fluorinated | acetonitrile (AN) | 0.1 vol. %-5 vol. % |
| Ester 2 | | |
| Fluorinated | acetonitrile (AN) | >5 vol. %-25 vol. % |
| Ester 2 | | |
| Fluorinated | acetonitrile (AN) | >25 vol. %-95 vol. % |
| Ester 2 | | |
| Fluorinated | fluoroacetonitrile | 0.1 vol. %-5 vol. % |
| Ester 2 | | |
| Fluorinated | fluoroacetonitrile | >5 vol. %-25 vol. % |
| Ester 2 | | |
| Fluorinated | fluoroacetonitrile | >25 vol. %-95 vol. % |
| Ester 2 | | |
| Fluorinated | ethyl acetate (EA) | 0.1 vol. %-5 vol. % |
| Ester 2 | | |
| Fluorinated | ethyl acetate (EA) | >5 vol. %-25 vol. % |
| Ester 2 | | |
| Fluorinated | ethyl acetate (EA) | >25 vol. %-95 vol. % |
| Ester 2 | | |
| Fluorinated | methyl acetate (MA) | 0.1 vol. %-5 vol. % |
| Ester 2 | | |
| Fluorinated | methyl acetate (MA) | >5 vol. %-25 vol. % |
| Ester 2 | | |
| Fluorinated | methyl acetate (MA) | >25 vol. %-95 vol. % |
| Ester 2 | | |
| Fluorinated | methyl propanoate (MP) | 0.1 vol. %-5 vol. % |
| Ester 2 | | |
| Fluorinated | methyl propanoate (MP) | >5 vol. %-25 vol. % |
| Ester 2 | | |
| Fluorinated | methyl propanoate (MP) | >25 vol. %-95 vol. % |
| Ester 2 | | |
| Fluorinated | methyl 3,3,3-trifluoropropanoate | 0.1 vol. %-5 vol. % |
| Ester 2 | | |
| Fluorinated | methyl 3,3,3-trifluoropropanoate | >5 vol. %-25 vol. % |
| Ester 2 | | |
| Fluorinated | methyl 3,3,3-trifluoropropanoate | >25 vol. %-95 vol. % |
| Ester 2 | | |
| Fluorinated | ethyl 3,3,3-trifluoropropanoate | 0.1 vol. %-5 vol. % |
| Ester 2 | | |
| Fluorinated | ethyl 3,3,3-trifluoropropanoate | >5 vol. %-25 vol. % |
| Ester 2 | | |
| Fluorinated | ethyl 3,3,3-trifluoropropanoate | >25 vol. %-95 vol. % |
| Ester 2 | | |
| Fluorinated | ethyl 2,2-difluoropropanoate | 0.1 vol. %-5 vol. % |
| Ester 2 | | |
| Fluorinated | ethyl 2,2-difluoropropanoate | >5 vol. %-25 vol. % |
| Ester 2 | | |
| Fluorinated | ethyl 2,2-difluoropropanoate | >25 vol. %-95 vol. % |
| Ester 2 | | |
| Fluorinated | isopropyl trifluoroacetate | 0.1 vol. %-5 vol. % |
| Ester 2 | | |
| Fluorinated | isopropyl trifluoroacetate | >5 vol. %-25 vol. % |
| Ester 2 | | |
| Fluorinated | isopropyl trifluoroacetate | >25 vol. %-95 vol. % |
| Ester 2 | | |
| Fluorinated | methyl 4,4,4-trifluorobutanoate | 0.1 vol. %-5 vol. % |
| Ester 2 | | |
| Fluorinated | methyl 4,4,4-trifluorobutanoate | >5 vol. %-25 vol. % |
| Ester 2 | | |
| Fluorinated | methyl 4,4,4-trifluorobutanoate | >25 vol. %-95 vol. % |
| Ester 2 | | |
| Fluorinated | methyl difluoroacetate | 0.1 vol. %-5 vol. % |
| Ester 2 | | |
| Fluorinated | methyl difluoroacetate | >5 vol. %-25 vol. % |
| Ester 2 | | |
| Fluorinated | methyl difluoroacetate | >25 vol. %-95 vol. % |
| Ester 2 | | |
| Fluorinated | 2,2,2-trifluoroethyl butanoate | 0.1 vol. %-5 vol. % |
| Ester 2 | | |
| Fluorinated | 2,2,2-trifluoroethyl butanoate | >5 vol. %-25 vol. % |
| Ester 2 | | |
| Fluorinated | 2,2,2-trifluoroethyl butanoate | >25 vol. %-95 vol. % |
| Ester 2 | | |
| Fluorinated | methyl 2,2-difluoropropanoate | 0.1 vol. %-5 vol. % |
| Ester 2 | | |
| Fluorinated | methyl 2,2-difluoropropanoate | >5 vol. %-25 vol. % |
| Ester 2 | | |
| Fluorinated | methyl 2,2-difluoropropanoate | >25 vol. %-95 vol. % |
| Ester 2 | | |
| Fluorinated | ethyl trifluoroacetate | 0.1 vol. %-5 vol. % |
| Ester 2 | | |
| Fluorinated | ethyl trifluoroacetate | >5 vol. %-25 vol. % |
| Ester 2 | | |
| Fluorinated | ethyl trifluoroacetate | >25 vol. %-95 vol. % |
| Ester 2 | | |
| Fluorinated | 2,2-difluoroethyl trifluoroacetate | 0.1 vol. %-5 vol. % |
| Ester 2 | | |
| Fluorinated | 2,2-difluoroethyl trifluoroacetate | >5 vol. %-25 vol. % |
| Ester 2 | | |
| Fluorinated | 2,2-difluoroethyl trifluoroacetate | >25 vol. %-95 vol. % |
| Ester 2 | | |
| Fluorinated | 2,2,2-trifluoroethyl acetate | 0.1 vol. %-5 vol. % |
| Ester 2 | | |
| Fluorinated | 2,2,2-trifluoroethyl acetate | >5 vol. %-25 vol. % |
| Ester 2 | | |
| Fluorinated | 2,2,2-trifluoroethyl acetate | >25 vol. %-95 vol. % |
| Ester 2 | | |
| Fluorinated | ethyl 2,2-difluoroacetate | 0.1 vol. %-5 vol. % |
| Ester 2 | | |
| Fluorinated | ethyl 2,2-difluoroacetate | >5 vol. %-25 vol. % |
| Ester 2 | | |
| Fluorinated | ethyl 2,2-difluoroacetate | >25 vol. %-95 vol. % |
| Ester 2 | | |
| Fluorinated | 2,2-difluoroethyl acetate | 0.1 vol. %-5 vol. % |
| Ester 2 | | |
| Fluorinated | 2,2-difluoroethyl acetate | >5 vol. %-25 vol. % |
| Ester 2 | | |
| Fluorinated | 2,2-difluoroethyl acetate | >25 vol. %-95 vol. % |
| Ester 2 | | |
| Fluorinated | 2,2-difluoroethyl | 0.1 vol. %-5 vol. % |
| Ester 2 | 3,3-difluoropropanoate | |
| Fluorinated | 2,2-difluoroethyl | >5 vol. %-25 vol. % |
| Ester 2 | 3,3-difluoropropanoate | |
| Fluorinated | 2,2-difluoroethyl | >25 vol. %-95 vol. % |
| Ester 2 | 3,3-difluoropropanoate | |
| Fluorinated | 2,2-difluoroethyl | 0.1 vol. %-5 vol. % |
| Ester 2 | 2,2-difluoroacetate | |
| Fluorinated | 2,2-difluoroethyl | >5 vol. %-25 vol. % |
| Ester 2 | 2,2-difluoroacetate | |
| Fluorinated | 2,2-difluoroethyl | >25 vol. %-95 vol. % |
| Ester 2 | 2,2-difluoroacetate | |
| Fluorinated | succinonitrile (SN) | 0.1 vol. %-5 vol. % |
| Ester 2 | | |
| Fluorinated | succinonitrile (SN) | >5 vol. %-25 vol. % |
| Ester 2 | | |
| Fluorinated | succinonitrile (SN) | >25 vol. %-95 vol. % |
| Ester 2 | | |
| Fluorinated | propionitrile (PN) | 0.1 vol. %-5 vol. % |
| Ester 2 | | |
| Fluorinated | propionitrile (PN) | >5 vol. %-25 vol. % |
| Ester 2 | | |
| Fluorinated | propionitrile (PN) | >25 vol. %-95 vol. % |
| Ester 2 | | |
| Fluorinated | butyronitrile (BN) | 0.1 vol. %-5 vol. % |
| Ester 2 | | |
| Fluorinated | butyronitrile (BN) | >5 vol. %-25 vol. % |
| Ester 2 | | |
| Fluorinated | butyronitrile (BN) | >25 vol. %-95 vol. % |
| Ester 2 | | |
| Fluorinated | adiponitrile (ADN) | 0.1 vol. %-5 vol. % |
| Ester 2 | | |
| Fluorinated | adiponitrile (ADN) | >5 vol. %-25 vol. % |
| Ester 2 | | |
| Fluorinated | adiponitrile (ADN) | >25 vol. %-95 vol. % |
| Ester 2 | | |
| Fluorinated | 1,3,6-hexanetricarbonitrile (HTCN) | 0.1 vol. %-5 vol. % |
| Ester 2 | | |
| Fluorinated | 1,3,6-hexanetricarbonitrile (HTCN) | >5 vol. %-25 vol. % |
| Ester 2 | | |
| Fluorinated | 1,3,6-hexanetricarbonitrile (HTCN) | >25 vol. %-95 vol. % |
| Ester 2 | | |
| Fluorinated | trimethyl borate (TMB) | 0.1 vol. %-5 vol. % |
| Ester 2 | | |
| Fluorinated | trimethyl borate (TMB) | >5 vol. %-25 vol. % |
| Ester 2 | | |
| Fluorinated | trimethyl borate (TMB) | >25 vol. %-95 vol. % |
| Ester 2 | | |
| Fluorinated | triphenyl borate (TPB) | 0.1 vol. %-5 vol. % |
| Ester 2 | | |
| Fluorinated | triphenyl borate (TPB) | >5 vol. %-25 vol. % |
| Ester 2 | | |
| Fluorinated | triphenyl borate (TPB) | >25 vol. %-95 vol. % |
| Ester 2 | | |
| Fluorinated | triethyl borate (TEB) | 0.1 vol. %-5 vol. % |
| Ester 2 | | |
| Fluorinated | triethyl borate (TEB) | >5 vol. %-25 vol. % |
| Ester 2 | | |
| Fluorinated | triethyl borate (TEB) | >25 vol. %-95 vol. % |
| Ester 2 | | |
| Fluorinated | tris(pentafluorophenyl)borane | 0.1 vol. %-5 vol. % |
| Ester 2 | (TPFPB) | |
| Fluorinated | tris(pentafluorophenyl)borane | >5 vol. %-25 vol. % |
| Ester 2 | (TPFPB) | |
| Fluorinated | tris(pentafluorophenyl)borane | >25 vol. %-95 vol. % |
| Ester 2 | (TPFPB) | |
| Fluorinated | tris(trimethylsilyl)phosphate | 0.1 vol. %-5 vol. % |
| Ester 2 | (TTSB) | |
| Fluorinated | tris(trimethylsilyl)phosphate | >5 vol. %-25 vol. % |
| Ester 2 | (TTSB) | |
| Fluorinated | tris(trimethylsilyl)phosphate | >25 vol. %-95 vol. % |
| Ester 2 | (TTSB) | |
| Fluorinated | tris(2,2,2-trifluoroethyl) borate | 0.1 vol. %-5 vol. % |
| Ester 2 | (TTFEB) | |
| Fluorinated | tris(2,2,2-trifluoroethyl) borate | >5 vol. %-25 vol. % |
| Ester 2 | (TTFEB) | |
| Fluorinated | tris(2,2,2-trifluoroethyl) borate | >25 vol. %-95 vol. % |
| Ester 2 | (TTFEB) | |
| Fluorinated | trimethyl phosphate (TMP) | 0.1 vol. %-5 vol. % |
| Ester 2 | | |
| Fluorinated | trimethyl phosphate (TMP) | >5 vol. %-25 vol. % |
| Ester 2 | | |
| Fluorinated | trimethyl phosphate (TMP) | >25 vol. %-95 vol. % |
| Ester 2 | | |
| Fluorinated | triethyl phosphate (TEP) | 0.1 vol. %-5 vol. % |
| Ester 2 | | |
| Fluorinated | triethyl phosphate (TEP) | >5 vol. %-25 vol. % |
| Ester 2 | | |
| Fluorinated | triethyl phosphate (TEP) | >25 vol. %-95 vol. % |
| Ester 2 | | |
| Fluorinated | tris(trimethylsilyl)phosphate | 0.1 vol. %-5 vol. % |
| Ester 2 | (TTSP) | |
| Fluorinated | tris(trimethylsilyl)phosphate | >5 vol. %-25 vol. % |
| Ester 2 | (TTSP) | |
| Fluorinated | tris(trimethylsilyl)phosphate | >25 vol. %-95 vol. % |
| Ester 2 | (TTSP) | |
| Fluorinated | tris(trimethylsilyl)phosphite | 0.1 vol. %-5 vol. % |
| Ester 2 | (TTSPi) | |
| Fluorinated | tris(trimethylsilyl)phosphite | >5 vol. %-25 vol. % |
| Ester 2 | (TTSPi) | |
| Fluorinated | tris(trimethylsilyl)phosphite | >25 vol. %-95 vol. % |
| Ester 2 | (TTSPi) | |
| Fluorinated | tris(2,2,2-trifluoroethyl) | 0.1 vol. %-5 vol. % |
| Ester 2 | phosphate (TFEPa) | |
| Fluorinated | tris(2,2,2-trifluoroethyl) | >5 vol. %-25 vol. % |
| Ester 2 | phosphate (TFEPa) | |
| Fluorinated | tris(2,2,2-trifluoroethyl) | >25 vol. %-95 vol. % |
| Ester 2 | phosphate (TFEPa) | |
| Fluorinated | tris(2,2,2-trifluoroethyl) | 0.1 vol. %-5 vol. % |
| Ester 2 | phosphite (TFEPi) | |
| Fluorinated | tris(2,2,2-trifluoroethyl) | >5 vol. %-25 vol. % |
| Ester 2 | phosphite (TFEPi) | |
| Fluorinated | tris(2,2,2-trifluoroethyl) | >25 vol. %-95 vol. % |
| Ester 2 | phosphite (TFEPi) | |
| Fluorinated | (pentafluorophenyl)diphenyl | 0.1 vol. %-5 vol. % |
| Ester 2 | phosphine (PFPDPP) | |
| Fluorinated | (pentafluorophenyl)diphenyl | >5 vol. %-25 vol. % |
| Ester 2 | phosphine (PFPDPP) | |
| Fluorinated | (pentafluorophenyl)diphenyl | >25 vol. %-95 vol. % |
| Ester 2 | phosphine (PFPDPP) | |
| Fluorinated | tris(pentafluorophenyl) | 0.1 vol. %-5 vol. % |
| Ester 2 | phosphine (TPFPP) | |
| Fluorinated | tris(pentafluorophenyl) | >5 vol. %-25 vol. % |
| Ester 2 | phosphine (TPFPP) | |
| Fluorinated | tris(pentafluorophenyl) | >25 vol. %-95 vol. % |
| Ester 2 | phosphine (TPFPP) | |
| Fluorinated | 1,3,2-dioxathiolane-2,2-dioxide | 0.1 vol. %-5 vol. % |
| Ester 2 | (DTD) | |
| Fluorinated | 1,3,2-dioxathiolane-2,2-dioxide | >5 vol. %-25 vol. % |
| Ester 2 | (DTD) | |
| Fluorinated | 1,3,2-dioxathiolane-2,2-dioxide | >25 vol. %-95 vol. % |
| Ester 2 | (DTD) | |
| Fluorinated | 1,3-propanesultone (PS) | 0.1 vol. %-5 vol. % |
| Ester 2 | | |
| Fluorinated | 1,3-propanesultone (PS) | >5 vol. %-25 vol. % |
| Ester 2 | | |
| Fluorinated | 1,3-propanesultone (PS) | >25 vol. %-95 vol. % |
| Ester 2 | | |
| Fluorinated | prop-1-ene-1,3-sultone (PES) | 0.1 vol. %-5 vol. % |
| Ester 2 | | |
| Fluorinated | prop-1-ene-1,3-sultone (PES) | >5 vol. %-25 vol. % |
| Ester 2 | | |
| Fluorinated | prop-1-ene-1,3-sultone (PES) | >25 vol. %-95 vol. % |
| Ester 2 | | |
| Fluorinated | propanediol cyclic sulfate (PCS) | 0.1 vol. %-5 vol. % |
| Ester 2 | | |
| Fluorinated | propanediol cyclic sulfate (PCS) | >5 vol. %-25 vol. % |
| Ester 2 | | |
| Fluorinated | propanediol cyclic sulfate (PCS) | >25 vol. %-95 vol. % |
| Ester 2 | | |
| Fluorinated | 1,3,2-dioxathiolane 2-oxide | 0.1 vol. %-5 vol. % |
| Ester 2 | | |
| Fluorinated | 1,3,2-dioxathiolane 2-oxide | >5 vol. %-25 vol. % |
| Ester 2 | | |
| Fluorinated | 1,3,2-dioxathiolane 2-oxide | >25 vol. %-95 vol. % |
| Ester 2 | | |
| Fluorinated | bis(2,2,2-trifluoroethyl) ether | 0.1 vol. %-5 vol. % |
| Ester 2 | (BTFE) | |
| Fluorinated | bis(2,2,2-trifluoroethyl) ether | >5 vol. %-25 vol. % |
| Ester 2 | (BTFE) | |
| Fluorinated | bis(2,2,2-trifluoroethyl) ether | >25 vol. %-95 vol. % |
| Ester 2 | (BTFE) | |
| Fluorinated | 1,1,2,2-tetrafluoroethyl- | 0.1 vol. %-5 vol. % |
| Ester 2 | 2,2,3,3-tetrafluoropropyl ether | |
| | (TTE) | |
| Fluorinated | 1,1,2,2-tetrafluoroethyl- | >5 vol. %-25 vol. % |
| Ester 2 | 2,2,3,3-tetrafluoropropyl ether | |
| | (TTE) | |
| Fluorinated | 1,1,2,2-tetrafluoroethyl- | >25 vol. %-95 vol. % |
| Ester 2 | 2,2,3,3-tetrafluoropropyl ether | |
| | (TTE) | |
| Fluorinated | 1H,1H,5H-octafluoropentyl- | 0.1 vol. %-5 vol. % |
| Ester 2 | 1,1,2,2-tetrafluoroethylether (OTE) | |
| Fluorinated | 1H,1H,5H-octafluoropentyl- | >5 vol. %-25 vol. % |
| Ester 2 | 1,1,2,2-tetrafluoroethylether (OTE) | |
| Fluorinated | 1H,1H,5H-octafluoropentyl- | >25 vol. %-95 vol. % |
| Ester 2 | 1,1,2,2-tetrafluoroethylether (OTE) | |
| Fluorinated | 2,2,2-trifluoroethyl 1,1,2,2- | 0.1 vol. %-5 vol. % |
| Ester 2 | tetrafluoroethyl ether (HFE) | |
| Fluorinated | 2,2,2-trifluoroethyl 1,1,2,2- | >5 vol. %-25 vol. % |
| Ester 2 | tetrafluoroethyl ether (HFE) | |
| Fluorinated | 2,2,2-trifluoroethyl 1,1,2,2- | >25 vol. %-95 vol. % |
| Ester 2 | tetrafluoroethyl ether (HFE) | |
| Fluorinated | tetrahydrofuran (THF) | 0.1 vol. %-5 vol. % |
| Ester 2 | | |
| Fluorinated | tetrahydrofuran (THF) | >5 vol. %-25 vol. % |
| Ester 2 | | |
| Fluorinated | tetrahydrofuran (THF) | >25 vol. %-95 vol. % |
| Ester 2 | | |
| Fluorinated | 1,3-dioxolane (DOL) | 0.1 vol. %-5 vol. % |
| Ester 2 | | |
| Fluorinated | 1,3-dioxolane (DOL) | >5 vol. %-25 vol. % |
| Ester 2 | | |
| Fluorinated | 1,3-dioxolane (DOL) | >25 vol. %-95 vol. % |
| Ester 2 | | |
| Fluorinated | 1,2-dimethoxyethane (DME) | 0.1 vol. %-5 vol. % |
| Ester 2 | | |
| Fluorinated | 1,2-dimethoxyethane (DME) | >5 vol. %-25 vol. % |
| Ester 2 | | |
| Fluorinated | 1,2-dimethoxyethane (DME) | >25 vol. %-95 vol. % |
| Ester 2 | | |
| Fluorinated | diethylene glycol dimethyl ether | 0.1 vol. %-5 vol. % |
| Ester 2 | (DEGDME) | |
| Fluorinated | diethylene glycol dimethyl ether | >5 vol. %-25 vol. % |
| Ester 2 | (DEGDME) | |
| Fluorinated | diethylene glycol dimethyl ether | >25 vol. %-95 vol. % |
| Ester 2 | (DEGDME) | |
| Fluorinated | tetraethylene glycol dimethyl | 0.1 vol. %-5 vol. % |
| Ester 2 | ether (TEGDME) | |
| Fluorinated | tetraethylene glycol dimethyl | >5 vol. %-25 vol. % |
| Ester 2 | ether (TEGDME) | |
| Fluorinated | tetraethylene glycol dimethyl | >25 vol. %-95 vol. % |
| Ester 2 | ether (TEGDME) | |
| Fluorinated | succinic anhydride (SA) | 0.1 vol. %-5 vol. % |
| Ester 2 | | |
| Fluorinated | succinic anhydride (SA) | >5 vol. %-25 vol. % |
| Ester 2 | | |
| Fluorinated | succinic anhydride (SA) | >25 vol. %-95 vol. % |
| Ester 2 | | |
| Fluorinated | butyric anhydride (BA) | 0.1 vol. %-5 vol. % |
| Ester 2 | | |
| Fluorinated | butyric anhydride (BA) | >5 vol. %-25 vol. % |
| Ester 2 | | |
| Fluorinated | butyric anhydride (BA) | >25 vol. %-95 vol. % |
| Ester 2 | | |
| Fluorinated | 1,4-dioxane (DOX) | 0.1 vol. %-5 vol. % |
| Ester 2 | | |
| Fluorinated | 1,4-dioxane (DOX) | >5 vol. %-25 vol. % |
| Ester 2 | | |
| Fluorinated | 1,4-dioxane (DOX) | >25 vol. %-95 vol. % |
| Ester 2 | | |
| Fluorinated | tetravinyl silane (TVSI) | 0.1 vol. %-5 vol. % |
| Ester 2 | | |
| Fluorinated | tetravinyl silane (TVSI) | >5 vol. %-25 vol. % |
| Ester 2 | | |
| Fluorinated | tetravinyl silane (TVSI) | >25 vol. %-95 vol. % |
| Ester 2 | | |
| Fluorinated | 1,4-butane sultone (BS) | 0.1 vol. %-5 vol. % |
| Ester 2 | | |
| Fluorinated | 1,4-butane sultone (BS) | >5 vol. %-25 vol. % |
| Ester 2 | | |
| Fluorinated | 1,4-butane sultone (BS) | >25 vol. %-95 vol. % |
| Ester 2 | | |
| Fluorinated | dimethyl sulfoxide (DMSO) | 0.1 vol. %-5 vol. % |
| Ester 2 | | |
| Fluorinated | dimethyl sulfoxide (DMSO) | >5 vol. %-25 vol. % |
| Ester 2 | | |
| Fluorinated | dimethyl sulfoxide (DMSO) | >25 vol. %-95 vol. % |
| Ester 2 | | |
| Fluorinated | methylene methanedisulfonate | 0.1 vol. %-5 vol. % |
| Ester 2 | (MMDS) | |
| Fluorinated | methylene methanedisulfonate | >5 vol. %-25 vol. % |
| Ester 2 | (MMDS) | |
| Fluorinated | methylene methanedisulfonate | >25 vol. %-95 vol. % |
| Ester 2 | (MMDS) | |
| Fluorinated | N,N-dimethylformamide (DMF) | 0.1 vol. %-5 vol. % |
| Ester 2 | | |
| Fluorinated | N,N-dimethylformamide (DMF) | >5 vol. %-25 vol. % |
| Ester 2 | | |
| Fluorinated | N,N-dimethylformamide (DMF) | >25 vol. %-95 vol. % |
| Ester 2 | | |
| Fluorinated | gamma-butyrolactone (BL) | 0.1 vol. %-5 vol. % |
| Ester 2 | | |
| Fluorinated | gamma-butyrolactone (BL) | >5 vol. %-25 vol. % |
| Ester 2 | | |
| Fluorinated | gamma-butyrolactone (BL) | >25 vol. %-95 vol. % |
| Ester 2 | | |

An electrolyte including at least one of Fluorinated Esters 1 and 2 is prepared by dissolving at least one of Lithium Salt 1-16 (for formation of a lithium-ion battery) or at least one of Sodium Salt 1-13 (for formation of a sodium-ion battery) at a concentration of 0.01 M-5 M in a solvent mixture of at least one of Fluorinated Esters 1 and 2 and optionally at least one supplemental solvent from Tables 7A-7D. The amount of the fluorinated ester in the electrolyte is 0.1-95 wt % and the amount of the at least one supplemental solvent in the electrolyte is 0-99 wt. % or 0.1-99 wt. %, respectively, based on a total weight of the electrolyte. The amount of the fluorinated ester in the electrolyte is 0.1-95 vol % and the amount of the at least one supplemental solvent in the electrolyte is 0-99 vol. % or 0.1-99 vol. %, respectively, based on a total volume of the electrolyte. Dissolving can be aided by application of heat, agitation, or a combination thereof.

### C. Additives

The electrolyte can further include one or more additives in addition to at least one of Fluorinated Esters 1 and 2 and a metal salt. The additive can be, for example, a conductive additive, an overcharge protection additive, a flame-retardant additive, a solid electrolyte interphase (SEI) film-forming additive, a cathode electrolyte interphase (CEI) film forming additive, a cathode material protective agent, metal salt stabilizer, or a combination thereof.

### V. Battery Formation

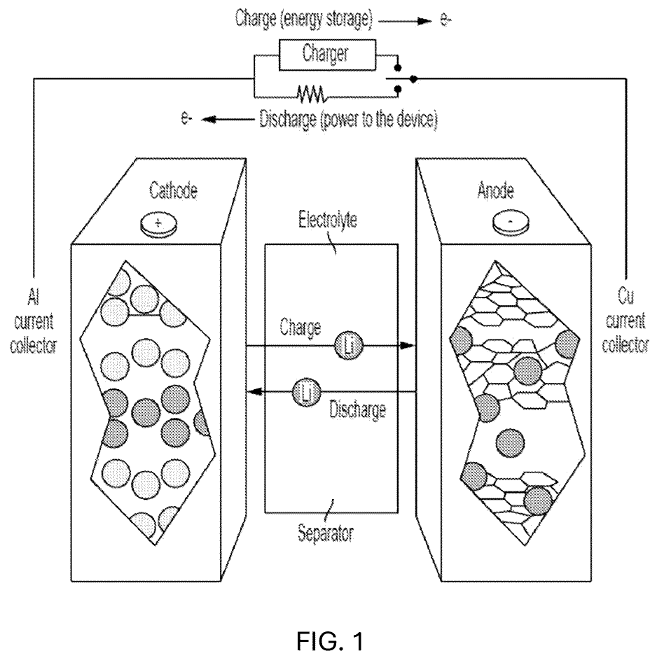

The present disclosure provides batteries and rechargeable batteries including electrolytes, which include at least one of Fluorinated Esters 1 and 2, an anode, a cathode, and a separator. An exemplary rechargeable battery is illustrated in FIG. 1, which shows a cathode, an anode, a porous separator, and electrolyte of the present disclosure which facilitates the flow of ions, for example, lithium or sodium ions, between the cathode and the anode.

### A. Anode

Table 8 provides anode materials that can be used in a battery including an electrolyte including at least one lithium salt and at least one of Fluorinated Esters 1 and 2. Table 9 provides anode materials that can be used in a battery including an electrolyte including at least one sodium salt and at least one of Fluorinated Esters 1 and 2. The anode material can be selected depending on a battery being formed and uses thereof. It is understood that the invention comprises, consists essentially of, or consists of the compositions identified in Table 8. It is understood that the invention comprises, consists essentially of, or consists of the compositions identified in Table 9.

TABLE 8

| Fluorinated Ester | Anode Material |
|---|---|
| Fluorinated Ester 1 | graphite |
| Fluorinated Ester 1 | lithium metal |
| Fluorinated Ester 1 | silicon |
| Fluorinated Ester 1 | silicon oxide (SiOₓ) |
| Fluorinated Ester 1 | graphite/silicon composite |
| Fluorinated Ester 1 | graphite/silicon oxide composite |
| Fluorinated Ester 1 | silicon/carbon composite |
| Fluorinated Ester 1 | silicon carbide |
| Fluorinated Ester 1 | graphite/silicon carbide composite |
| Fluorinated Ester 1 | silicon nitride |
| Fluorinated Ester 1 | graphite/silicon nitride composite |
| Fluorinated Ester 1 | hard carbon |
| Fluorinated Ester 1 | amorphous carbon |
| Fluorinated Ester 1 | lithium titanium oxide (LTO) |
| Fluorinated Ester 2 | graphite |
| Fluorinated Ester 2 | lithium metal |
| Fluorinated Ester 2 | silicon |
| Fluorinated Ester 2 | silicon oxide (SiOₓ) |
| Fluorinated Ester 2 | graphite/silicon composite |
| Fluorinated Ester 2 | graphite/silicon oxide composite |
| Fluorinated Ester 2 | silicon/carbon composite |
| Fluorinated Ester 2 | silicon carbide |
| Fluorinated Ester 2 | graphite/silicon carbide composite |
| Fluorinated Ester 2 | silicon nitride |
| Fluorinated Ester 2 | graphite/silicon nitride composite |
| Fluorinated Ester 2 | hard carbon |
| Fluorinated Ester 2 | amorphous carbon |
| Fluorinated Ester 2 | lithium titanium oxide (LTO) |

TABLE 9

| Fluorinated Ester | Anode Material |
|---|---|
| Fluorinated Ester 1 | sodium metal |
| Fluorinated Ester 1 | graphite |
| Fluorinated Ester 1 | silicon |
| Fluorinated Ester 1 | silicon oxide (SiOₓ) |
| Fluorinated Ester 1 | graphite/silicon composite |
| Fluorinated Ester 1 | graphite/silicon oxide composite |
| Fluorinated Ester 1 | silicon/carbon composite |
| Fluorinated Ester 1 | silicon carbide |
| Fluorinated Ester 1 | graphite/silicon carbide composite |
| Fluorinated Ester 1 | silicon nitride |
| Fluorinated Ester 1 | graphite/silicon nitride composite |
| Fluorinated Ester 1 | hard carbon |
| Fluorinated Ester 1 | amorphous carbon |
| Fluorinated Ester 1 | sodium titanium oxide (NTO) |
| Fluorinated Ester 1 | tin |
| Fluorinated Ester 1 | tin oxide |
| Fluorinated Ester 1 | tin sulfide |
| Fluorinated Ester 2 | sodium metal |
| Fluorinated Ester 2 | graphite |
| Fluorinated Ester 2 | silicon |
| Fluorinated Ester 2 | silicon oxide (SiOₓ) |
| Fluorinated Ester 2 | graphite/silicon composite |
| Fluorinated Ester 2 | graphite/silicon oxide composite |
| Fluorinated Ester 2 | silicon/carbon composite |
| Fluorinated Ester 2 | silicon carbide |
| Fluorinated Ester 2 | graphite/silicon carbide composite |
| Fluorinated Ester 2 | silicon nitride |
| Fluorinated Ester 2 | graphite/silicon nitride composite |
| Fluorinated Ester 2 | hard carbon |
| Fluorinated Ester 2 | amorphous carbon |
| Fluorinated Ester 2 | sodium titanium oxide (NTO) |
| Fluorinated Ester 2 | tin |
| Fluorinated Ester 2 | tin oxide |
| Fluorinated Ester 2 | tin sulfide |

### B. Cathode

Table 10 provides cathode materials that can be used in a battery including an electrolyte including at least one lithium salt and at least one of Fluorinated Esters 1 and 2. Table 11 provides cathode materials that can be used in a battery including an electrolyte including at least one sodium salt and at least one of Fluorinated Esters 1 and 2. The cathode material can be selected depending on a battery being formed and uses thereof. It is understood that the invention comprises, consists essentially of, or consists of the compositions identified in Table 10. It is understood that the invention comprises, consists essentially of, or consists of the compositions identified in Table 11.

TABLE 10

| Fluorinated Ester | Cathode Material |
|---|---|
| Fluorinated Ester 1 | lithium nickel manganese cobalt oxide (NMC) |
| Fluorinated Ester 1 | lithium nickel cobalt aluminum oxide (NCA) |
| Fluorinated Ester 1 | lithium nickel manganese aluminum oxide (NMA) |
| Fluorinated Ester 1 | lithium nickel manganese cobalt aluminum |
| | oxide (NMCA) |
| Fluorinated Ester 1 | lithium nickel oxide (LNO) |
| Fluorinated Ester 1 | lithium nickel manganese oxide (LiNi_{0.5}Mn_{1.5}O₄) |
| Fluorinated Ester 1 | lithium cobalt oxide (LCO) |
| Fluorinated Ester 1 | lithium manganese oxide (LMO) |
| Fluorinated Ester 1 | lithium and manganese-rich cathode (LMR |
| | or LLMO) |
| Fluorinated Ester 1 | lithium iron phosphate (LFP) |
| Fluorinated Ester 1 | lithium cobalt phosphate (LCP) |
| Fluorinated Ester 1 | lithium manganese phosphate (LMP) |
| Fluorinated Ester 1 | lithium manganese iron phosphate (LMFP) |
| Fluorinated Ester 1 | transition metal sulfide |
| Fluorinated Ester 1 | sulfur |
| Fluorinated Ester 1 | sulfur/carbon composite |
| Fluorinated Ester 2 | lithium nickel manganese cobalt oxide (NMC) |
| Fluorinated Ester 2 | lithium nickel cobalt aluminum oxide (NCA) |
| Fluorinated Ester 2 | lithium nickel manganese aluminum oxide (NMA) |
| Fluorinated Ester 2 | lithium nickel manganese cobalt aluminum |
| | oxide (NMCA) |
| Fluorinated Ester 2 | lithium nickel oxide (LNO) |
| Fluorinated Ester 2 | lithium nickel manganese oxide (LiNi_{0.5}Mn_{1.5}O₄) |
| Fluorinated Ester 2 | lithium cobalt oxide (LCO) |
| Fluorinated Ester 2 | lithium manganese oxide (LMO) |
| Fluorinated Ester 2 | lithium and manganese-rich cathode (LMR |
| | or LLMO) |
| Fluorinated Ester 2 | lithium iron phosphate (LFP) |
| Fluorinated Ester 2 | lithium cobalt phosphate (LCP) |
| Fluorinated Ester 2 | lithium manganese phosphate (LMP) |
| Fluorinated Ester 2 | lithium manganese iron phosphate (LMFP) |
| Fluorinated Ester 2 | transition metal sulfide |
| Fluorinated Ester 2 | sulfur |
| Fluorinated Ester 2 | sulfur/carbon composite |

TABLE 11

| Fluorinated Ester | Cathode Material |
|---|---|
| Fluorinated Ester 1 | sodium vanadium phosphate |
| Fluorinated Ester 1 | sodium copper nickel iron manganese oxide |
| Fluorinated Ester 1 | Prussian white |
| Fluorinated Ester 1 | Prussian blue |
| Fluorinated Ester 1 | sodium iron manganese oxide |
| Fluorinated Ester 1 | sodium nickel manganese oxide |
| Fluorinated Ester 1 | sodium ferric phosphate pyrophosphate |
| Fluorinated Ester 1 | sodium vanadium phosphate pyrophosphate |
| Fluorinated Ester 1 | sodium ferric sulfate |
| Fluorinated Ester 1 | sodium manganese oxide |
| Fluorinated Ester 1 | sodium manganese phosphate |
| Fluorinated Ester 1 | sodium nickel manganese iron oxide |
| Fluorinated Ester 1 | sodium iron nickel oxide |
| Fluorinated Ester 1 | sodium iron phosphate |
| Fluorinated Ester 1 | sodium iron fluorophosphate |
| Fluorinated Ester 1 | sodium iron manganese fluorophosphate |
| Fluorinated Ester 1 | sodium vanadium fluorophosphate |
| Fluorinated Ester 1 | sodium iron pyrophosphate |
| Fluorinated Ester 1 | sodium manganese pyrophosphate |
| Fluorinated Ester 1 | sodium vanadium pyrophosphate |
| Fluorinated Ester 1 | transition metal sulfide |
| Fluorinated Ester 1 | sulfur |
| Fluorinated Ester 1 | sulfur/carbon composite |
| Fluorinated Ester 2 | sodium vanadium phosphate |
| Fluorinated Ester 2 | sodium copper nickel iron manganese oxide |
| Fluorinated Ester 2 | Prussian white |
| Fluorinated Ester 2 | Prussian blue |
| Fluorinated Ester 2 | sodium iron manganese oxide |
| Fluorinated Ester 2 | sodium nickel manganese oxide |
| Fluorinated Ester 2 | sodium ferric phosphate pyrophosphate |
| Fluorinated Ester 2 | sodium vanadium phosphate pyrophosphate |
| Fluorinated Ester 2 | sodium ferric sulfate |
| Fluorinated Ester 2 | sodium manganese oxide |
| Fluorinated Ester 2 | sodium manganese phosphate |
| Fluorinated Ester 2 | sodium nickel manganese iron oxide |
| Fluorinated Ester 2 | sodium iron nickel oxide |
| Fluorinated Ester 2 | sodium iron phosphate |
| Fluorinated Ester 2 | sodium iron fluorophosphate |
| Fluorinated Ester 2 | sodium iron manganese fluorophosphate |
| Fluorinated Ester 2 | sodium vanadium fluorophosphate |
| Fluorinated Ester 2 | sodium iron pyrophosphate |
| Fluorinated Ester 2 | sodium manganese pyrophosphate |
| Fluorinated Ester 2 | sodium vanadium pyrophosphate |
| Fluorinated Ester 2 | transition metal sulfide |
| Fluorinated Ester 2 | sulfur |
| Fluorinated Ester 2 | sulfur/carbon composite |

A battery is formed with an electrolyte from Tables 3 and 4 (for formation of a lithium-ion battery) or Table 5 and 6 (for formation of a sodium-ion battery), optionally including a, e.g., at least one, supplemental solvent in Tables 7A-7D, an anode including an anode material from Table 8 (for formation of a lithium-ion battery) or Table 9 (for formation of a sodium-ion battery), and a cathode including a cathode material from Table 10 (for formation of a lithium-ion battery) or Table 11 (for formation of a sodium-ion battery). Depending on properties of the battery, the battery is prepared in ambient conditions, a dry room, or an argon-filled glove box. The battery is prepared as a coin cell, pouch cell, cylindrical cell, or prismatic cell form factor with a nominal capacity of 10 milliampere-hours (mAh)-1,000 ampere-hours (Ah). Electrolyte is added to each cell.

### VI. Battery Performance

Electrochemical cycling testing is carried out using a battery tester with temperature controlled at −60° C.-60° C. The battery is first subjected to formation cycles at C/100-1 C charge rate and C/100-1 C discharge rate with a lower voltage limit of 0 volts (V)-3 V and upper voltage limit of 1.5 V-6.0 V. As used herein, a C-rate means a current which will discharge a battery in one hour, e.g., a C-rate for a battery having a discharge capacity of 1.6 ampere-hours would be 1.6 amperes.

The battery is subjected to extended cycling using a C/100-1 C charge rate and C/100-1 C discharge rate. Electrochemical performance parameters of the battery include the number of cycles to reach 80% of initial capacity and the battery resistance growth from electrochemical impedance spectroscopy (EIS) measurements. Batteries with the electrolyte formulation exhibit a higher initial (dis) charge capacity, higher number of cycles before reaching 80% capacity retention, reduced impedance, or a combination thereof compared to a comparative example.

### EXAMPLES

### Synthetic Methods

Provided herein are methods of preparing fluorinated esters, as illustrated by the following examples.

### Synthetic Example 1: Fluorinated Ester 1 (Difluoromethyl 3,3,3-trifluoropropanoate)

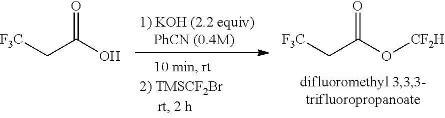 [SMILES: O=C(O)CC(F)(F)F ; O=C(CC(F)(F)F)OC(F)F]

An oven-dried two-neck flask equipped with a magnetic stir bar and a thermocouple is charged with KOH (0.1 moles (mol), 6.17 grams (g), 2.20 equivalents (equiv.)) (ground just prior to addition) and benzonitrile (PhCN) (125 milliliters (mL)). The flask is sealed with a septum, placed in an ice bath, and 3,3,3-trifluoropropanoic acid (0.05 mol, 4.4 mL, 1 equiv.) is added dropwise maintaining the temperature between 20-25° C. The ice bath is removed, and the mixture stirred at room temperature for 10 minutes. After this time, the flask is placed again in an ice bath and (Bromodifluoromethyl) trimethylsilane (TMSCF₂Br) (0.1 mol, 15.6 mL, 2.0 equiv.) is added dropwise, maintaining the temperature at 0° C. Once the addition is finalized, the ice bath is removed, and the reaction mix is stirred at room temperature for 2 hours. The flask is then opened, filtered, and the filtrate subjected to distillation to afford the expected compound.

### Synthetic Example 2: Fluorinated Ester 2 (2.2-difluornethvl 3.3.3-triflunronronannate)

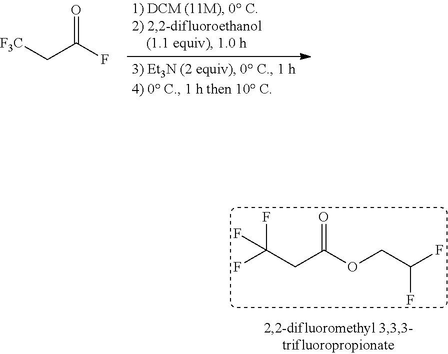 [SMILES: O=C(F)CC(F)(F)F ; O=C(CC(F)(F)F)OCC(F)F]

In a two-neck round bottom flask equipped with a stir bar and a thermocouple, 3,3,3-trifluoropropanoyl fluoride (0.05 mol, 6.5 mL, 1 equiv.) is dissolved in 25 mL of dichloromethane to obtain a dichloromethane solution of acetyl fluoride. This solution is cooled to 0° C. and then 2,2-difluoroethanol (0.055 mol, 3.47 mL, 1.1 equiv.) is added via syringe pump over 1 hour (h), maintaining the 0° C. temperature. After this time, triethylamine (0.115 mol, 2.3 equiv.) is added dropwise in 3 batches with stirring between the additions for 1 h. After the completion of the dropwise addition, the reaction is maintained at 0° C. for an additional hour, and then sodium carbonate (0.18 mol) is added as a solid and the stirring of the mixture is continued at 0° C. for 1 h. The mixture is slowly heated to 10° C. and left to react until the difluoroethanol is completely consumed. Next, 200 ml of water are added, and the organic phase is collected and dried over anhydrous magnesium and distilled to obtain the desired ester.

### Example Set 3A (Lithium-Ion Electrolyte Formulation)

An electrolyte including difluoromethyl 3,3,3-trifluoropropanoate (Fluorinated Ester 1) is prepared by dissolving lithium hexafluorophosphate (LiPF₆) (Lithium Salt 4) at 1 moles per liter (molar, M) concentration in Fluorinated Ester 1. This example is repeated substituting Fluorinated Ester 1 with Fluorinated Ester 2. Each of the previous examples in this example set is repeated using a 1:1 (vol:vol) mixture of Fluorinated Ester 1 and Fluorinated Ester 2. Each of the previous examples in this example set is repeated substituting Lithium Salt 4 with each of Lithium Salts 1-3 and 5-16. Each of the previous examples in this example set is repeated substituting the 1M concentration of the lithium salt with each of 0.05M, 0.5M, and 2M concentrations.

### Example Set 3B (Lithium-Ion Battery Formation)

Each electrolyte of Example Set 3A is tested in a battery with a graphite anode, a lithium nickel manganese cobalt oxide (NMC) cathode, and a polypropylene separator. The battery is prepared in an argon-filled glove box and has a pouch cell form factor with a nominal capacity of 300 mAh. An amount of 1 mL of electrolyte is added to each cell. Each of the previous examples in this example set is repeated substituting the graphite anode with each of the other anodes in Table 8. Each of the previous examples in this example set is repeated substituting the NMC cathode with each of the other cathodes in Table 10.

### Example Set 3C (Lithium-Ion Electrolyte Formulations Including Supplemental Solvents)

An electrolyte including difluoromethyl 3,3,3-trifluoropropanoate (Fluorinated Ester 1) is prepared by dissolving lithium hexafluorophosphate (LiPF₆) (Lithium Salt 4) at 1 moles per liter (molar, M) concentration in a solvent mixture of Fluorinated Ester 1 and fluoroethylene carbonate (FEC). The amount of Fluorinated Ester 1 in the solvent mixture is 10 weight percent (wt. %) and the amount of fluoroethylene carbonate in the solvent mixture is 90 wt. %, based on a total weight of the solvent mixture. This example is repeated substituting Fluorinated Ester 1 with Fluorinated Ester 2. Each of the previous examples in this example set is repeated using a 1:1 (vol: vol) mixture of Fluorinated Ester 1 and Fluorinated Ester 2. Each of the previous examples in this example set is repeated substituting Lithium Salt 4 with each of Lithium Salts 1-3 and 5-16. Each of the previous examples in this example set is repeated substituting the 1M concentration of the lithium salt with each of 0.05M, 0.5M, and 2M concentrations. Each of the previous examples in this example set is repeated substituting the fluorinated ester: supplemental solvent ratio of 1:9 with a 1:19 ratio. Each of the previous examples in this example set is repeated substituting the fluorinated ester: supplemental solvent ratio with a 9:1 ratio. Each of the previous examples in this example set is repeated substituting FEC with each of the supplemental solvents from Tables 7A-7D. Selected exemplary lithium-ion formulations are detailed in Table 12.

TABLE 12
Exemplary lithium-ion electrolyte formulations

| | | | Fluorinated | | |
|---|---|---|---|---|---|
| | | | Ester: | | |
| | | | Supplementary | | Salt |
| Exam- | Fluorinated | Supplementary | Solvent | | Conc. |
| ple | Ester | Solvent | ratio (w:w) | Salt | (M) |
| 3C-A | Fluorinated | Fluoroethylene | 9:1 | LiPF₆ | 1 |
| | Ester 1 | carbonate | | | |
| 3C-B | Fluorinated | Difluoroethylene | 9:1 | LiPF₆ | 1 |
| | Ester 1 | carbonate | | | |
| 3C-C | Fluorinated | Fluoroethylene | 9:1 | LiPF₆ | 1 |
| | Ester 2 | carbonate | | | |
| 3C-D | Fluorinated | Difluoroethylene | 9:1 | LiPF₆ | 1 |
| | Ester 2 | carbonate | | | |
| 3C-E | Fluorinated | Fluoroethylene | 9:1 | LiFSI | 1 |
| | Ester 2 | carbonate | | | |

### Example Set 3D (Lithium-Ion Battery Formation)

Each electrolyte of Example Set 3C is tested in a battery with a graphite anode and lithium nickel manganese cobalt oxide (NMC) cathode. The battery is prepared in an argon-filled glove box and has a pouch cell form factor with a nominal capacity of 300 mAh. An amount of 1 mL of electrolyte is added to each cell. Each of the previous examples in this example set is repeated substituting the graphite anode with each of the other anodes in Table 8. Each of the previous examples in this example set is repeated substituting the NMC cathode with each of the other cathodes in Table 10. Selected exemplary lithium-ion batteries are detailed in Table 13.

TABLE 13
Exemplary lithium-ion batteries

| Battery | | |
|---|---|---|
| Example | Anode Material | Cathode material |
| 3D-A | graphite | lithium nickel manganese cobalt oxide |
| 3D-B | graphite | lithium nickel manganese oxide |
| 3D-C | graphite | lithium manganese iron phosphate |
| 3D-D | graphite | lithium and manganese-rich |
| | | cathode |
| 3D-E | graphite/silicon | lithium nickel manganese cobalt oxide |
| | composite | |

### Example 3E (Lithium-Ion Electrolyte Formulation)

An electrolyte including difluoromethyl 3,3,3-trifluoropropanoate (Fluorinated Ester 1) is prepared by dissolving lithium hexafluorophosphate (LiPF₆) (Lithium Salt 4) at 1 moles per liter (molar, M) concentration in a solvent mixture of difluoromethyl 3,3,3-trifluoropropanoate (Fluorinated Ester 1) and fluoroethylene carbonate (FEC). The amount of difluoromethyl 3,3,3-trifluoropropanoate in the solvent mixture is 90 weight percent (wt. %) and the amount of fluoroethylene carbonate in the solvent mixture is 10 wt. %, based on a total weight of the solvent mixture. Further examples of electrolyte formulation include substitution of Fluorinated Ester 1 with Fluorinated Ester 2, addition to Fluorinated Ester 1 of Fluorinated Ester 2, substitution of Lithium Salt 4 with at least one of Lithium Salts 1-3 or 5-16, addition to Lithium Salt 4 of at least one of Lithium Salts 1-3 or 5-16 (see Tables 3 and 4), omission of FEC, substitution of FEC with a different supplemental solvent in Tables 7A-7D, addition to FEC of a different supplemental solvent in Tables 7A-7D, or a combination thereof.

### Example 3F (Lithium-Ion Battery Formation)

The electrolyte of Example 3E is tested in a battery with a graphite anode and lithium nickel manganese cobalt oxide (NMC) cathode. The battery is prepared in an argon-filled glove box and has a pouch cell form factor with a nominal capacity of 300 mAh. An amount of 1 mL of electrolyte is added to each cell. Further examples of battery formation include substitution of the graphite anode with a different anode material in Table 8, substitution of the NMC cathode with a different cathode material in Table 10, or a combination thereof.

### Example 3G (Lithium-Ion Battery Performance)

Electrochemical cycling tests of the battery of Example 3F is carried out using a Maccor, Inc. battery tester with temperature controlled at 40° C. The battery is first subjected to 3 formation cycles at C/20 charge rate and C/20 discharge rate with a lower voltage limit of 3.0 V and upper voltage limit of 4.5 V. After completion of formation cycles, the battery is degassed and re-sealed in the argon filled glove box.

The battery is subjected to extended cycling using a 0.5 C charge rate and 0.5 C discharge rate. Electrochemical performance parameters of the battery include the number of cycles to reach 80% of initial capacity and the battery resistance growth from electrochemical impedance spectroscopy (EIS) measurements. Batteries with the electrolyte formulation exhibit more cycles before reaching 80% capacity retention compared to batteries utilizing a 1 M LiPF6 3:7 ethylene carbonate: dimethyl carbonate electrolyte.

### Example 4A (Lithium-Ion Electrolyte Formulation)

Lithium hexafluorophosphate (LiPF₆) (Lithium Salt 4) at 1 M concentration is dissolved in a 9:1 volume ratio mixture of at least one of Fluorinated Esters 1 and 2:fluoroethylene carbonate (FEC). Further examples of electrolyte formulation include substitution of Lithium Salt 4 with at least one of Lithium Salts 1-3 or 5-16, addition to Lithium Salt 4 of at least one of Lithium Salts 1-3 or 5-16 (see Tables 3 and 4), substitution of FEC with a different supplemental solvent in Tables 7A-7D, addition to FEC of a different supplemental solvent in Tables 7A-7D, or a combination thereof.

### Example 4B (Lithium Ion Battery Formation)

The electrolyte of Example 4A is tested in a battery with a graphite anode, lithium nickel manganese cobalt oxide (NMC) cathode, and polypropylene separator. The battery is prepared in an argon-filled glove box and has a pouch cell form factor with a nominal capacity of 300 mAh. Further examples of battery formation include substitution of the graphite anode with a different anode material in Table 8, substitution of the NMC cathode with a different cathode material in Table 10, or a combination thereof.

### Example 4C (Lithium-Ion Battery Performance)

The electrochemical cycling tests of the battery of Example 4B are carried out using a Maccor Inc. battery tester with temperature control at 40° C. The battery is first subjected to three formation cycles at C/20 rate (charge and discharge over a period of 20 hours). After completion of formation cycles, the battery is degassed and re sealed in the argon filled glove box. The battery is subjected to extended cycling using a 0.5 C charge rate and 0.5 C discharge rate. Electrochemical performance parameters of the battery include the number of cycles to reach 80% of initial capacity and the battery resistance growth from electrochemical impedance spectroscopy (EIS) measurements. Batteries with the electrolyte formulation exhibit more cycles before reaching 80% capacity retention compared to batteries utilizing a 1 M LiPF6 3:7 ethylene carbonate: dimethyl carbonate electrolyte.

### Example Set 5A (Sodium-Ion Electrolyte Formulation)

An electrolyte including difluoromethyl 3,3,3-trifluoropropanoate (Fluorinated Ester 1) is prepared by dissolving sodium hexafluorophosphate (NaPF₆) (Sodium Salt 4) at 1 moles per liter (molar, M) concentration in Fluorinated Ester 1. This example is repeated substituting Fluorinated Ester 1 with Fluorinated Ester 2. Each of the previous examples in this example set is repeated using a 1:1 (vol:vol) mixture of Fluorinated Ester 1 and Fluorinated Ester 2. Each of the previous examples in this example set is repeated substituting Sodium Salt 4 with each of Sodium Salts 1-3 and 5-13. Each of the previous examples in this example set is repeated substituting the 1M concentration of the sodium salt with each of 0.05M, 0.5M, and 2M concentrations.

### Example Set 5B (Sodium-Ion Battery Formations)

Each electrolyte of Example Set 5A is tested in a battery with a hard carbon anode, a and sodium nickel manganese iron oxide cathode. The battery is prepared in an argon-filled glove box and has a pouch cell form factor with a nominal capacity of 300 mAh. An amount of 1 mL of electrolyte is added to each cell. Each of the previous examples in this example set is repeated substituting the hard carbon anode with each of the other anodes in Table 9. Each of the previous examples in this example set is repeated substituting the sodium nickel manganese iron oxide cathode with each of the other cathodes in Table 11.

### Example Set 5C (Sodium-Ion Electrolyte Formulations Including Supplemental Solvents)

An electrolyte including difluoromethyl 3,3,3-trifluoropropanoate (Fluorinated Ester 1) is prepared by dissolving sodium hexafluorophosphate (NaPF₆) (Sodium Salt 4) at 1 moles per liter (molar, M) concentration in a solvent mixture of Fluorinated Ester 1 and fluoroethylene carbonate (FEC). The amount of difluoromethyl 3,3,3-trifluoropropanoate in the solvent mixture is 10 weight percent (wt. %) and the amount of fluoroethylene carbonate in the solvent mixture is 90 wt. %, based on a total weight of the solvent mixture. This example is repeated substituting Fluorinated Ester 1 with Fluorinated Ester 2. Each of the previous examples in this example set is repeated using a 1:1 (vol: vol) mixture of Fluorinated Ester 1 and Fluorinated Ester 2. Each of the previous examples in this example set is repeated substituting Sodium Salt 4 with each of Sodium Salts 1-3 and 5-13. Each of the previous examples in this example set is repeated substituting the 1M concentration of the sodium salt with each of 0.05M, 0.5M, and 2M concentrations. Each of the previous examples in this example set is repeated substituting the fluorinated ester: supplemental solvent ratio of 1:9 with a 1:19 ratio. Each of the previous examples in this example set is repeated substituting the fluorinated ester: supplemental solvent ratio with a 9:1 ratio. Each of the previous examples in this example set is repeated substituting FEC with each of the supplemental solvents from Tables 7A-7D. Selected exemplary sodium-ion formulations are detailed in Table 14.

TABLE 14
Exemplary sodium-ion electrolyte formulations

| | | | Fluorinated | | |
|---|---|---|---|---|---|
| | | | Ester: | | |
| | | | Supplementary | | Salt |
| Exam- | Fluorinated | Supplementary | Solvent | | Conc. |
| ple | Ester | Solvent | ratio (w:w) | Salt | (M) |
| 5C-A | Fluorinated | Fluoroethylene | 9:1 | NaP | 1 |
| | Ester 1 | carbonate | | F₆ | |
| 5C-B | Fluorinated | Difluoroethylene | 9:1 | NaP | 1 |
| | Ester 1 | carbonate | | F₆ | |
| 5C-C | Fluorinated | Fluoroethylene | 9:1 | NaP | 1 |
| | Ester 2 | carbonate | | F₆ | |
| 5C-D | Fluorinated | Difluoroethylene | 9:1 | NaP | 1 |
| | Ester 2 | carbonate | | F₆ | |
| 5C-E | Fluorinated | Fluoroethylene | 9:1 | NaF | 1 |
| | Ester 2 | carbonate | | SI | |

### Example Set 5D (Sodium-Ion Battery Formation)

Each electrolyte of Example Set 5C is tested in a battery with a hard carbon anode and sodium nickel manganese iron oxide cathode. The battery is prepared in an argon-filled glove box and has a pouch cell form factor with a nominal capacity of 300 mAh. An amount of 1 mL of electrolyte is added to each cell. Each of the previous examples in this example set is repeated substituting the hard carbon anode with each of the other anodes in Table 9. Each of the previous examples in this example set is repeated substituting the sodium nickel manganese iron oxide cathode with each of the other cathodes in Table 11. Selected exemplary sodium-ion batteries are detailed in Table 15.

TABLE 15
Exemplary sodium-ion batteries

| Battery Example | Anode Material | Cathode material |
|---|---|---|
| 5D-A | hard carbon | sodium iron pyrophosphate |
| 5D-B | hard carbon | sodium nickel manganese iron oxide |
| 5D-C | hard carbon | sodium nickel manganese oxide |
| 5D-D | hard carbon | sodium copper nickel iron manganese |
| | | oxide |
| 5D-E | tin | sodium nickel manganese iron oxide |

### Example 5E (Sodium-Ion Electrolyte Formulation)

An electrolyte including difluoromethyl 3,3,3-trifluoropropanoate (Fluorinated Ester 1) is prepared by dissolving sodium hexafluorophosphate (NaPF₆) (Sodium Salt 4) at 1 M concentration in a solvent mixture of difluoromethyl 3,3,3-trifluoropropanoate (Fluorinated Ester 1) and fluoroethylene carbonate (FEC). The amount of difluoromethyl 3,3,3-trifluoropropanoate in the solvent mixture is 90 weight percent (wt. %) and the amount of fluoroethylene carbonate in the solvent mixture is 10 wt. %, based on a total weight of the solvent mixture. Further examples of electrolyte formulation include substitution of Fluorinated Ester 1 with Fluorinated Ester 2, addition to Fluorinated Ester 1 of Fluorinated Ester 2, substitution of Sodium Salt 4 with at least one of Sodium Salts 1-3 or 5-13, addition to Sodium Salt 4 of at least one of Sodium Salts 1-3 or 5-13 (see Tables 5 and 6), omission of FEC, substitution of FEC with a different supplemental solvent in Tables 7A-7D, addition to FEC of a different supplemental solvent in Tables 7A-7D, or a combination thereof.

### Example 5F (Sodium-Ion Battery Formation)

The electrolyte of Example 5E is tested in a battery with a hard carbon anode and sodium nickel manganese iron oxide cathode. The battery is prepared in an argon-filled glove box and has a pouch cell form factor with a nominal capacity of 200 mAh. An amount of 1 mL of electrolyte is added to each cell. Further examples of battery formation include substitution of the hard carbon anode with a different anode material in Table 9, substitution of the sodium nickel manganese iron oxide cathode with a different cathode material in Table 11, or a combination thereof.

### Example 5G (Sodium-Ion Battery Performance)

Electrochemical cycling tests of the battery of Example 5F is carried out using a Maccor, Inc. battery tester with temperature controlled at 40° C. The battery is first subjected to 3 formation cycles at C/20 charge rate and C/20 discharge rate with a lower voltage limit of 3.0 V and upper voltage limit of 4.0 V. After completion of formation cycles, the battery is degassed and re sealed in the argon filled glove box.

The battery is subjected to extended cycling using a 0.5 C charge rate and 0.5 C discharge rate. Electrochemical performance parameters of the battery include the number of cycles to reach 80% of initial capacity and the battery resistance growth from electrochemical impedance spectroscopy (EIS) measurements. Batteries with the electrolyte formulation exhibit more cycles before reaching 80% capacity retention compared to batteries utilizing a 1 M NaPF6 3:7 ethylene carbonate: dimethyl carbonate electrolyte.

### Example 6A (Sodium-Ion Electrolyte Formulation)

Sodium hexafluorophosphate (NaPF₆) (Sodium Salt 4) at 1 M concentration is dissolved in a 9:1 volume ratio mixture of at least one of Fluorinated Esters 1 and 2: fluoroethylene carbonate (FEC). Further examples of electrolyte formulation include substitution of Sodium Salt 4 with at least one of Sodium Salts 1-3 or 5-13, addition to Sodium Salt 4 of at least one of Sodium Salts 1-3 or 5-13 (see Tables 5 and 6), substitution of FEC with a different supplemental solvent in Tables 7A-7D, addition to FEC of a different supplemental solvent in Tables 7A-7D, or a combination thereof.

### Example 6B (Sodium-Ion Battery Formation)

The electrolyte of Example 6A is tested in a battery with a hard carbon anode, sodium nickel manganese iron oxide cathode, and polypropylene separator. The battery is prepared in an argon-filled glove box and has a pouch cell form factor with a nominal capacity of 300 mAh. Further examples of battery formation include substitution of the hard carbon anode with a different anode material in Table 9, substitution of the sodium nickel manganese iron oxide with a different cathode material in Table 11, or a combination thereof.

### Example 6C (Sodium-Ion Battery Performance)

The electrochemical cycling tests of the battery of Example 6B are carried out using a Maccor Inc. battery tester with temperature control at 40° C. The battery is first subjected to three formation cycles at C/20 rate (charge and discharge over a period of 20 hours). After completion of formation cycles, the battery is degassed and re sealed in the argon filled glove box. The battery is then subjected to extended cycling using a 0.5 C charge rate and 0.5 C discharge rate. Electrochemical performance parameters of the battery include the number of cycles to reach 80% of initial capacity and the battery resistance growth from electrochemical impedance spectroscopy (EIS) measurements. Batteries with the electrolyte formulation exhibit more cycles before reaching 80% capacity retention compared to batteries utilizing a 1 M NaPF6 3:7 ethylene carbonate: dimethyl carbonate electrolyte.

### Example 7: Synthesis of Fluorinated Ester 2 (2,2-difluoroethyl 3,3,3-trifluoropropanoate)

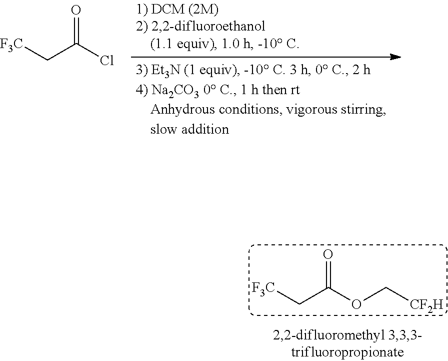 [SMILES: O=C(Cl)CC(F)(F)F ; O=C(CC(F)(F)F)OCC(F)F]

A dried two liter (L) three-neck round bottom flask equipped with an overhead stirrer, a thermocouple, and a soda lime trap was evacuated and refilled with nitrogen three times. Then, 500 mL of anhydrous dichloromethane was added via syringe under nitrogen, followed by trifluoropropanoyl chloride (1 mol, 101.03 mL, 1 equiv.) to obtain a dichloromethane solution of trifluoropropanoyl chloride. This solution was cooled to −10° C. and then 2,2-difluoroethanol (1.1 mol, 69.4 mL, 1.1 equiv.) was added via syringe pump over 1 hour. After this time, triethylamine (1 mol, 101.19 mL, 1 equiv.) was added dropwise with an addition funnel equipped with a metering valve over a period of 3 hours with vigorous stirring. The rate of addition was determined by the control of the exotherm. After the completion of the dropwise addition, the reaction was warmed to 0° C. and stirred for 2 more hours. Then, sodium carbonate (300 g) was added as a solid and the stirring of the mixture was continued at 0° C. for 1 h. The mixture was allowed to warm to room temperature and 500 mL of water was added to the reaction pot. The contents of the flask were then transferred into a separation funnel, and the organic layer was separated and washed 3 times with 10% HCl and extracted with dichloromethane until complete removal of triethylamine (approximately 700 mL total) and subsequently washed with 600 mL of saturated sodium bicarbonate. The organic layer was thoroughly dried with anhydrous magnesium sulfate, filtered and subjected to distillation.73% yield.

### Example 8: Synthesis of Fluorinated Ester 1 (difluoromethyl 3,3,3-trifluoropropanoate)

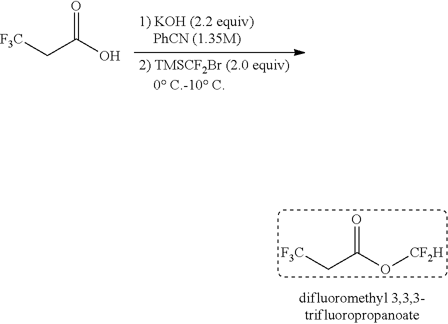 [SMILES: O=C(O)CC(F)(F)F ; O=C(CC(F)(F)F)OC(F)F]

A 250 mL three-neck round bottom flask equipped with a stir bar and a thermocouple is charged with hydroxide (KOH) (0.24 mol, 13.5 g, 2.20 equiv.) and benzonitrile (PhCN) (81 mL). The flask is sealed with a septum, placed in a bath at 0° C., and 3,3,3-trifluoropropanoic acid (0.11 mol, 9.7 mL, 1 equiv.) is added dropwise maintaining the internal temperature between 15-20° C. Then, the reaction is allowed to cool down and (Bromodifluoromethyl) trimethylsilane (TMSCF₂Br) (0.22 mol, 34.2 mL, 2.0 equiv.) is added dropwise with an addition funnel, maintaining the internal temperature between 0-10° C. Once the addition is finalized, the reaction mix is stirred at room temperature for 2 hours. Then, the contents of the flask are transferred into a separation funnel. The reaction mix is washed 3 times with saturated sodium bisulfite, 10% HCl, and saturated sodium bicarbonate. The organic layer is thoroughly dried with anhydrous magnesium sulfate, filtered and purified by distillation. 65% yield.

### Example 9A (Lithium Ion Electrolyte Formulation)

An electrolyte including difluoromethyl 3,3,3-trifluoropropanoate (Fluorinated Ester 1) was prepared by dissolving lithium hexafluorophosphate (Lithium Salt 4) at 0.77 M concentration in a solvent mixture of difluoromethyl 3,3,3-trifluoropropanoate (Fluorinated Ester 1) and fluoroethylene carbonate. The amount of difluoromethyl 3,3,3-trifluoropropanoate (Fluorinated Ester 1) in the solvent mixture was 76 volume percent (vol. %) and the amount of fluoroethylene carbonate in the solvent mixture was 24 vol. %, based the total volume of the solvent mixture. The ionic conductivity of the electrolyte was measured at 30° C. by electrochemical impedance spectroscopy (EIS) using coin cells filled with electrolyte, where stainless steel acted as both electrodes (top and bottom of coin cell) and a polytetrafluoroethylene (PTFE) ring was used to define the sample volume, See D. Di Lecce and J. Hassoun, ACS Omega, 3, 8583 (2018); V. Nilsson, A. Kotronia, M. Lacey, K. Edström, and P. Johansson, ACS Appl. Energy Mater., 3, 200 (2020); and Q. Liu, T. L. Dzwiniel, K. Z. Pupek, and Z. Zhang, J. Electrochem. Soc., 166, A3959 (2019). EIS was measured using a BioLogic VSP impedance analyzer with an amplitude of 5 millivolts (mV) over a frequency range of 1 to 10 megahertz (mHz). Ohmic resistance of the electrolyte was determined from the intersection with the Z′ axis at high frequency on a Nyquis plot of the impedance. The resistance (R) was used to calculate the conductivity (o) using the contact area of the electrolyte on the stainless steel electrodes(S) and the distance between the electrodes (D) according to equation 1.

$$σ = \frac{D}{R S} \qquad [ 1 ]$$

The measured ionic conductivity of the electrolyte was 5.27±0.03 milliSiemens per centimeter (mS/cm) at 30° C.

### Example 9B (Lithium Ion Battery Formation)

The electrolyte of Example 9A was used to form a lithium-ion battery. Dry and sealed pouch cells with lithium nickel manganese cobalt oxide (LiNi_{0.8}Mn_{0.1}Co_{0.1}O₂, NMC811) based cathodes and graphite based anodes were obtained from Novonix Limited (Bedford, Nova Scotia). The nominal capacity of the cells was 300 mAh. The cathodes included 94 weight percent (wt. %) active material and the single side coating active material electrode loading was 17.91 milligrams per square centimeter (mg/cm2). The anodes included 94.5 wt. % active material and the single side coating active material loading was 11.86 mg/cm2. The pouch cells were cut open and filled with 0.88 milliliters (mL) electrolyte in an argon filled glove box and sealed with a pouch sealer (MSK-115A Vacuum Sealing Machine) under vacuum at a pressure of −90 kilopascals (kPa) (relative to atmospheric pressure).

### Example 9C (Lithium Ion Battery Performance)

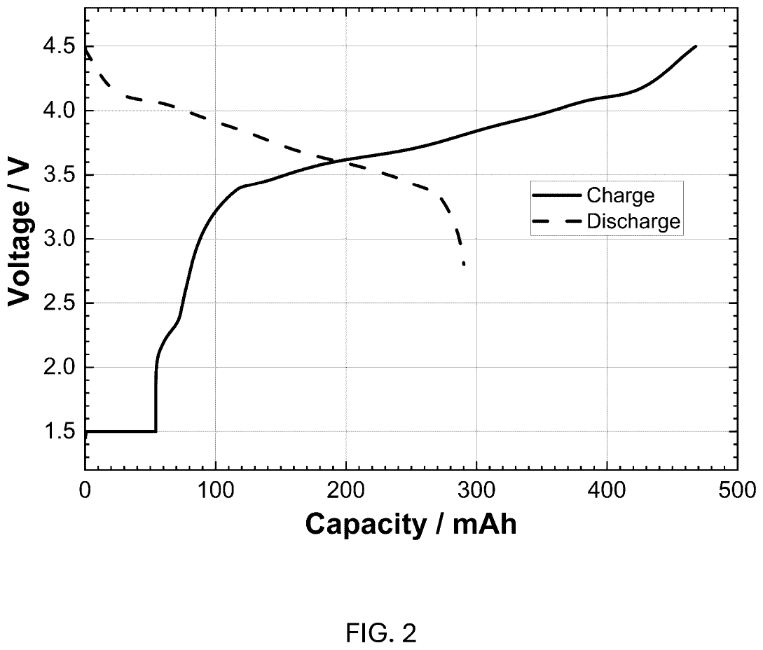

Electrochemical formation tests of the battery in Example 9A were carried out using a Maccor Inc. Series 4000 battery cycler and an environmental chamber controlled at 40° C. After filling with electrolyte as described in Example 8B, the pouch cells underwent an electrochemical formation protocol including the following steps: 1) Voltage hold at 1.5 volts (V) for 24 hours (h), 2) charge to 4.5 V at C/20 rate, 3) discharge to 2.8 V at C/20 rate, 4) charge to 4.5 V at C/20 rate, 5) open circuit voltage for 48 h, 6) discharge to 2.8 V at C/20 rate. The first discharge capacity of a representative example battery was 291 mAh and the first cycle coulombic efficiency of the battery was 55.5%. The voltage profile of the first charge and first discharge of a representative example battery is shown in FIG. 2.

### Example 10A (Lithium Ion Electrolyte Formulation)

An electrolyte including 2,2-difluoroethyl 3,3,3-trifluoropropanoate (Fluorinated Ester 2) was prepared by dissolving lithium hexafluorophosphate (Lithium Salt 4) at 1.0 M concentration in a solvent mixture of 2,2-difluoroethyl 3,3,3-trifluoropropanoate (Fluorinated Ester 2) and fluoroethylene carbonate. The amount of 2,2-difluoroethyl 3,3,3-trifluoropropanoate

(Fluorinated Ester 2) in the solvent mixture was 90 vol. % and the amount of fluoroethylene carbonate in the solvent mixture was 10 vol. %, based the total volume of the solvent mixture.

The ionic conductivity of the electrolyte was measured at 30° C. using the method described in Example 8A. The measured ionic conductivity of the electrolyte was 2.39 +0.03 mS/cm at 30° C.

### Example 10B (Lithium Ion Battery Formation)

The electrolyte of Example 10A was used to form a lithium-ion battery. Dry and sealed pouch cells with lithium nickel manganese cobalt oxide (LiNi_{0.8}Mn_{0.1}Co_{0.1}O₂, NMC811) based cathodes and graphite based anodes were obtained from Novonix Limited (Bedford, Nova Scotia). The nominal capacity of the cells was 300 mAh. The cathodes included 94 wt. % active material and the single side coating active material electrode loading was 17.91 mg/cm². The anodes included 94.5 wt. % active material and the single side coating active material loading was 11.86 mg/cm². The pouch cells were cut open and filled with 0.88 mL electrolyte in an argon filled glove box and sealed with a pouch sealer (MSK-115A Vacuum Sealing Machine) under vacuum at a pressure of −90 kPa (relative to atmospheric pressure).

### Example 10C (Lithium Ion Battery Performance)

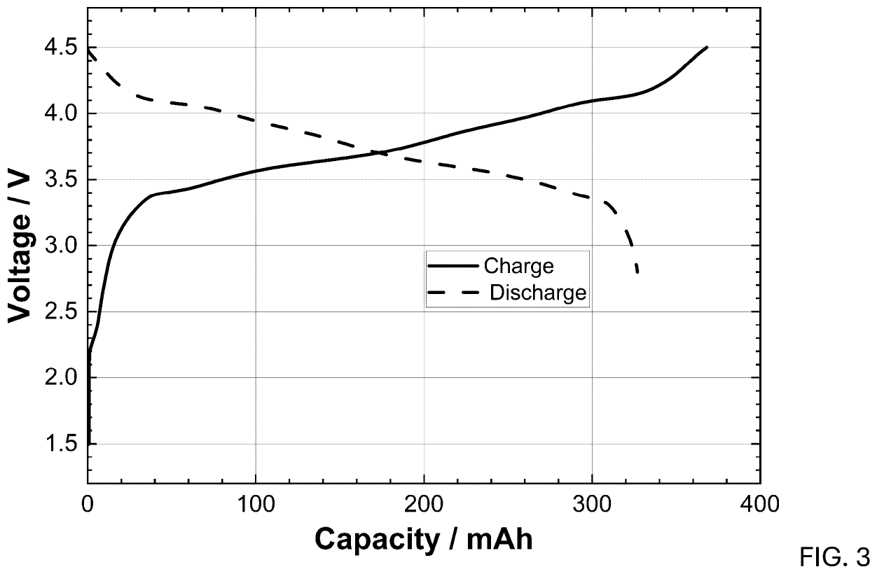

Electrochemical formation tests of the battery in Example 10A were carried out using a Maccor, Inc. Series 4000 battery cycler and a temperature controlled environmental chamber at 40° C. After filling with electrolyte as described in Example 10B, the cells underwent an electrochemical formation protocol including the following steps: 1) Voltage hold at 1.5 V for 24 h, 2) charge to 4.5 V at C/20 rate, 3) discharge to 2.8 V at C/20 rate, 4) charge to 4.5 V at C/20 rate, 5) open circuit voltage for 48 h, 6) discharge to 2.8 V at C/20 rate. The first discharge capacity of a representative example battery was 327 mAh and the first cycle coulombic efficiency of the battery was 88.8%. The voltage profile of the first charge and first discharge of a representative example battery is shown in FIG. 3.

After the formation step, the batteries were transferred and moved into the glove box, cut open to release gas, and then vacuum sealed again. Extended battery cycling was performed using a Maccor, Inc. Series 4000 battery cycler and an environmental chamber controlled at 40° C. Extended battery cycling was performed using a constant current constant voltage charging (CCCV) charge step and a constant current discharge step. The constant current constant voltage charging (CCCV) charge step included a C/5 charge constant current charge until 4.5 V was reached, followed by a constant voltage hold at 4.5 V for 20 minutes. The constant current discharge step used a C/5 discharge current until the lower limit voltage of 2.8 V was reached.

Cells filled with the electrolyte as described in Example 9A were compared to cells filled with a comparative electrolyte formulation. The comparative electrolyte was comprised of 1M lithium hexafluorophosphate (LiPF₆) dissolved in a solvent mixture of 30 wt. % ethylene carbonate (EC) and 70 wt. % ethyl methyl carbonate (EMC), with fluoroethylene carbonate (FEC) added at 2 wt. %, based on the total electrolyte weight. Cells utilizing the comparative electrolyte underwent the same electrolyte filling, electrochemical formation, and degassing procedures as described herein.

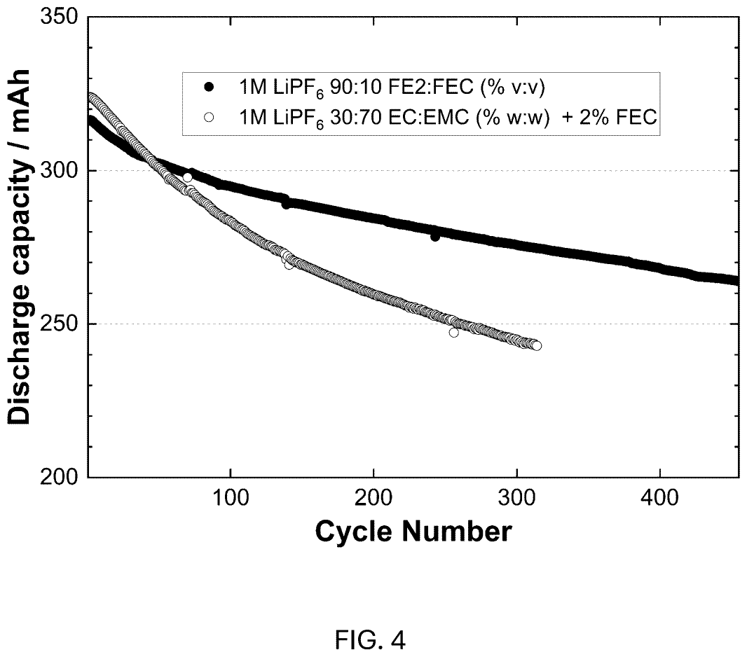

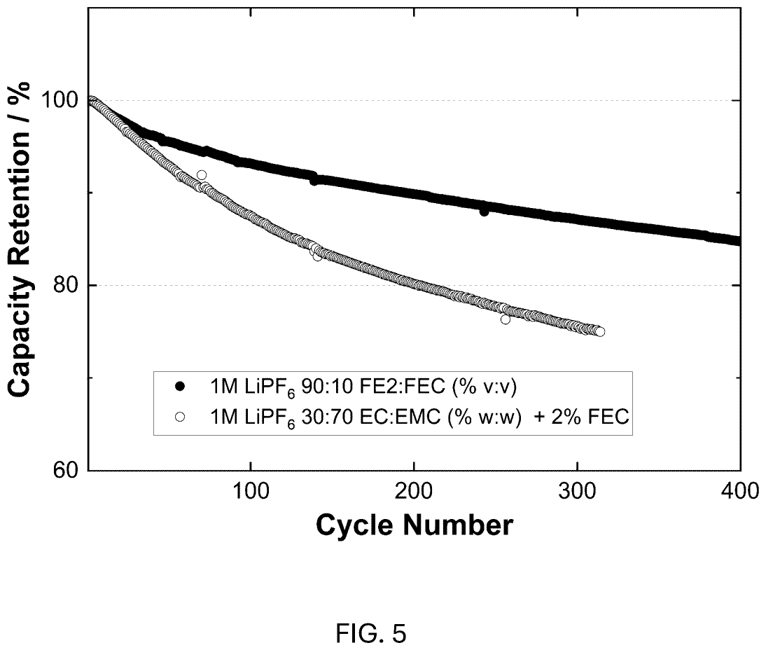

Representative extended cycling results are illustrated in FIG. 4 (discharge capacity versus cycle number for NMC811/graphite cells cycled between 2.8-4.5 V at 40°° C.) and FIG. 5 (capacity retention versus cycle number for NMC811/graphite cells cycled between 2.8-4.5 V at 40° C.). The cell utilizing the comparative electrolyte exhibit a higher discharge capacity during the first cycle (324 mAh versus 316 mAh for comparative electrolyte and Fluorinated Ester 2 based electrolyte, respectively) but also experienced a faster rate of capacity fade. The cell utilizing the comparative electrolyte fell to 80% capacity retention by cycle 202, while cells utilizing the Fluorinated Ester 2 based electrolyte maintain 85% capacity retention through 400 cycles. The data illustrate that using the Fluorinated Ester 2 electrolyte system described herein significantly improves capacity retention under operating conditions of 40° C. temperature and 4.5 V upper charge voltage limit.

### ASPECTS

This disclosure further encompasses the following aspects.

Aspect 1: A fluorinated ester selected from difluoromethyl 3,3,3-trifluoropropanoate or 2,2-difluoroethyl 3,3,3-trifluoropropanoate.

Aspect 2: A method of synthesizing a fluorinated ester, the method comprising: providing a carboxylic acid or carboxylic acid derivative; and reacting the carboxylic acid or carboxylic acid derivative with: a difluorocarbene; or a fluorinated alcohol; to synthesize the fluorinated ester.

Aspect 3: The method of aspect 2, wherein the carboxylic acid or carboxylic acid derivative is fluorinated.

Aspect 4: The method of aspect 2, wherein the difluorocarbene is generated from (bromodifluoromethyl) trimethylsilane.

Aspect 5: The method of aspect 2, wherein the carboxylic acid comprises 3,3,3-trifluoropropanoic acid.

Aspect 6: The method of aspect 2, wherein the carboxylic acid derivative comprises a derivative of 3,3,3-trifluoropropanoic acid.

Aspect 7: The method of aspect 2, wherein the carboxylic acid derivative comprises an acid fluoride, an acid chloride, or an acid bromide.

Aspect 8: The method of aspect 2, wherein the fluorinated alcohol comprises 2,2-difluoroethanol.

Aspect 9: A fluorinated ester synthesized by the method of aspect 2, wherein the fluorinated ester is difluoromethyl 3,3,3-trifluoropropanoate.

Aspect 10: A fluorinated ester synthesized by the method of aspect 2, wherein the fluorinated ester is 2,2-difluoroethyl 3,3,3-trifluoropropanoate.

Aspect 11: A method of synthesizing a fluorinated ester, the method comprising: providing a fluorinated carboxylic acid; and reacting the fluorinated carboxylic acid with difluorocarbene to synthesize the fluorinated ester.

Aspect 12: A fluorinated ester synthesized by the method of aspect 11, wherein the fluorinated ester is difluoromethyl 3,3,3-trifluoropropanoate.

Aspect 13: A method of synthesizing a fluorinated ester, the method comprising: providing a fluorinated acid fluoride; and reacting the fluorinated acid fluoride with a fluorinated alcohol to synthesize the fluorinated ester.

Aspect 14: A fluorinated ester synthesized by the method of aspect 13, wherein the fluorinated ester is 2,2-difluoroethyl 3,3,3-trifluoropropanoate.

Aspect 15: A method of synthesizing a fluorinated ester, the method comprising: providing a fluorinated acid halide (e.g., a fluorinated acid chloride or a fluorinated acid bromide); and reacting the fluorinated acid halide (e.g., the fluorinated acid chloride or the fluorinated acid bromide) with a fluorinated alcohol to synthesize the fluorinated ester.

Aspect 16: A fluorinated ester synthesized by the method of aspect 15, wherein the fluorinated ester is 2,2-difluoroethyl 3,3,3-trifluoropropanoate.

Aspect 17: An electrolyte comprising: difluoromethyl 3,3,3-trifluoropropanoate, 2,2-difluoroethyl 3,3,3-trifluoropropanoate, or a combination thereof; and lithium bis(trifluoromethanesulfonyl)imide, lithium bis(fluorosulfonyl)imide, lithium bis(oxalato)borate, lithium hexafluorophosphate, lithium tetrafluoroborate, lithium perchlorate, lithium hexafluoroarsenate, lithium triflate, lithium nitrate, lithium difluorophosphate, lithium difluoro(oxalato)borate, lithium trifluoroacetate, lithium 4,5-dicyano-2-(trifluoromethyl)imidazole, lithium difluoro(dioxalato)phosphate, lithium tetrafluoro(oxalato)phosphate, lithium bis(pentafluoroethanesulfonyl)imide, or a combination thereof.

Aspect 18: The electrolyte of aspect 17, further comprising fluoroethylene carbonate, propylene carbonate, dimethyl carbonate, diethyl carbonate, ethyl methyl carbonate, vinyl carbonate, vinyl ethylene carbonate, ethylene carbonate, difluoroethylene carbonate, 3,3,3-trifluoropropylene carbonate, monofluoroethyl methyl carbonate, difluoroethyl methyl carbonate, trifluoroethyl methyl carbonate, bis(2,2,2-trifluoroethyl) carbonate, 2,2-difluoroethyl ethyl carbonate, acetonitrile, fluoroacetonitrile, ethyl acetate, methyl acetate, methyl propanoate, methyl 3,3,3-trifluoropropanoate, ethyl 3,3,3-trifluoropropanoate, ethyl 2,2-difluoropropanoate, isopropyl trifluoroacetate, methyl 4,4,4-trifluorobutanoate, methyl difluoroacetate, 2,2,2-trifluoroethyl butanoate, methyl 2,2-difluoropropanoate, ethyl trifluoroacetate, 2,2-difluoroethyl trifluoroacetate, 2,2,2-trifluoroethyl acetate, ethyl 2,2-difluoroacetate, 2,2-difluoroethyl acetate, 2,2-difluoroethyl 3,3-difluoropropanoate, 2,2-difluoroethyl 2,2-difluoroacetate, succinonitrile, propionitrile, butyronitrile, adiponitrile, 1,3,6-hexanetricarbonitrile, trimethyl borate, triphenyl borate, triethyl borate, tris(pentafluorophenyl) borane, tris(trimethylsilyl)phosphate, tris(2,2,2-trifluoroethyl) borate, trimethyl phosphate, triethyl phosphate, tris(trimethylsilyl)phosphate, tris(trimethylsilyl)phosphite, tris(2,2,2-trifluoroethyl) phosphate, tris(2,2,2-trifluoroethyl)phosphite, (pentafluorophenyl)diphenyl phosphine, tris(pentafluorophenyl) phosphine, 1,3,2-dioxathiolane-2,2-dioxide, 1,3-propanesultone, prop-1-ene-1,3-sultone, propanediol cyclic sulfate, 1,3,2-dioxathiolane 2-oxide, bis(2,2,2-trifluoroethyl) ether, 1,1,2,2-tetrafluoroethyl-2,2,3,3-tetrafluoropropyl ether, 1H,1H,5H-octafluoropentyl-1,1,2,2-tetrafluoroethylether, 2,2,2-trifluoroethyl 1,1,2,2-tetrafluoroethyl ether, tetrahydrofuran, 1,3-dioxolane, 1,2-dimethoxyethane, diethylene glycol dimethyl ether, tetraethylene glycol dimethyl ether, succinic anhydride, butyric anhydride, 1,4-dioxane, tetravinyl silane, 1,4-butane sultone, dimethyl sulfoxide, methylene methanedisulfonate, N,N-dimethylformamide, gamma-butyrolactone, or a combination thereof.

Aspect 19: A battery comprising: the electrolyte of aspect 17 or 18; an anode comprising graphite, lithium metal, silicon, a silicon oxide, a graphite/silicon composite, a graphite/silicon oxide composite, a silicon/carbon composite, a silicon carbide, a graphite/silicon carbide composite, a silicon nitride, a graphite/silicon nitride composite, a hard carbon, an amorphous carbon, or lithium titanium oxide; and a cathode comprising lithium nickel manganese cobalt oxide, lithium nickel cobalt aluminum oxide, lithium nickel manganese aluminum oxide, lithium nickel manganese cobalt aluminum oxide, lithium nickel oxide, lithium nickel manganese oxide, lithium cobalt oxide, lithium manganese oxide, lithium and manganese-rich cathode, lithium iron phosphate, lithium cobalt phosphate, lithium manganese phosphate, lithium manganese iron phosphate, a transition metal sulfide, sulfur, or a sulfur/carbon composite.

Aspect 20: An electrolyte comprising: difluoromethyl 3,3,3-trifluoropropanoate, 2,2-difluoroethyl 3,3,3-trifluoropropanoate, or a combination thereof; and sodium bis(trifluoromethanesulfonyl)imide, sodium bis(fluorosulfonyl)imide, sodium bis(oxalato)borate, sodium hexafluorophosphate, sodium tetrafluoroborate, sodium perchlorate, sodium hexafluoroarsenate, sodium triflate, sodium nitrate, sodium difluorophosphate, sodium difluoro(oxalato)borate, sodium trifluoroacetate, sodium 4,5-dicyano-2-(trifluoromethyl)imidazole, or a combination thereof.

Aspect 21: The electrolyte of aspect 20, further comprising fluoroethylene carbonate, propylene carbonate, dimethyl carbonate, diethyl carbonate, ethyl methyl carbonate, vinyl carbonate, vinyl ethylene carbonate, ethylene carbonate, difluoroethylene carbonate, 3,3,3-trifluoropropylene carbonate, monofluoroethyl methyl carbonate, difluoroethyl methyl carbonate, trifluoroethyl methyl carbonate, bis(2,2,2-trifluoroethyl) carbonate, 2,2-difluoroethyl ethyl carbonate, acetonitrile, fluoroacetonitrile, ethyl acetate, methyl acetate, methyl propanoate, methyl 3,3,3-trifluoropropanoate, ethyl 3,3,3-trifluoropropanoate, ethyl 2,2-difluoropropanoate, isopropyl trifluoroacetate, methyl 4,4,4-trifluorobutanoate, methyl difluoroacetate, 2,2,2-trifluoroethyl butanoate, methyl 2,2-difluoropropanoate, ethyl trifluoroacetate, 2,2-difluoroethyl trifluoroacetate, 2,2,2-trifluoroethyl acetate, ethyl 2,2-difluoroacetate, 2,2-difluoroethyl acetate, 2,2-difluoroethyl 3,3-difluoropropanoate, 2,2-difluoroethyl 2,2-difluoroacetate, succinonitrile, propionitrile, butyronitrile, adiponitrile, 1,3,6-hexanetricarbonitrile, trimethyl borate, triphenyl borate, triethyl borate, tris(pentafluorophenyl)borane, tris(trimethylsilyl) phosphate, tris(2,2,2-trifluoroethyl) borate, trimethyl phosphate, triethyl phosphate, tris(trimethylsilyl) phosphate, tris(trimethylsilyl)phosphite, tris(2,2,2-trifluoroethyl) phosphate, tris(2,2,2-trifluoroethyl)phosphite, (pentafluorophenyl)diphenyl phosphine, tris(pentafluorophenyl) phosphine, 1,3,2-dioxathiolane-2,2-dioxide, 1,3-propanesultone, prop-1-ene-1,3-sultone, propanediol cyclic sulfate, 1,3,2-dioxathiolane 2-oxide, bis(2,2,2-trifluoroethyl) ether, 1,1,2,2-tetrafluoroethyl-2,2,3,3-tetrafluoropropyl ether, 1H,1H,5H-octafluoropentyl-1,1,2,2-tetrafluoroethylether, 2,2,2-trifluoroethyl 1,1,2,2-tetrafluoroethyl ether, tetrahydrofuran, 1,3-dioxolane, 1,2-dimethoxyethane, diethylene glycol dimethyl ether, tetraethylene glycol dimethyl ether, succinic anhydride, butyric anhydride, 1,4-dioxane, tetravinyl silane, 1,4-butane sultone, dimethyl sulfoxide, methylene methanedisulfonate, N,N-dimethylformamide, gamma-butyrolactone, or a combination thereof.

Aspect 22: A battery comprising: the electrolyte of aspect 20 or 21; an anode comprising sodium metal, graphite, silicon, a silicon oxide, a graphite/silicon composite, a graphite/silicon oxide composite, a silicon/carbon composite, a silicon carbide, a graphite/silicon carbide composite, a silicon nitride, a graphite/silicon nitride composite, a hard carbon, an amorphous carbon, sodium titanium oxide, tin, a tin oxide, or a tin sulfide; and a cathode comprising sodium vanadium phosphate, sodium copper nickel iron, manganese oxide, Prussian white, Prussian blue, sodium iron manganese oxide, sodium nickel manganese oxide, sodium ferric phosphate pyrophosphate, sodium vanadium phosphate pyrophosphate, sodium ferric sulfate, sodium manganese oxide, sodium manganese phosphate, sodium nickel manganese iron oxide, sodium iron nickel oxide, sodium iron phosphate, sodium iron fluorophosphate, sodium iron manganese fluorophosphate, sodium vanadium fluorophosphate, sodium iron pyrophosphate, sodium manganese pyrophosphate, sodium vanadium pyrophosphate, a transition metal sulfide, sulfur, or a sulfur/carbon composite.

Aspect 23: An electrolyte comprising: difluoromethyl 3,3,3-trifluoropropanoate, 2,2-difluoroethyl 3,3,3-trifluoropropanoate, or a combination thereof; fluoroethylene carbonate, propylene carbonate, dimethyl carbonate, diethyl carbonate, ethyl methyl carbonate, vinyl carbonate, vinyl ethylene carbonate, ethylene carbonate, difluoroethylene carbonate, 3,3,3-trifluoropropylene carbonate, monofluoroethyl methyl carbonate, difluoroethyl methyl carbonate, trifluoroethyl methyl carbonate, bis(2,2,2-trifluoroethyl) carbonate, 2,2-difluoroethyl ethyl carbonate, acetonitrile, fluoroacetonitrile, ethyl acetate, methyl acetate, methyl propanoate, methyl 3,3,3-trifluoropropanoate, ethyl 3,3,3-trifluoropropanoate, ethyl 2,2-difluoropropanoate, isopropyl trifluoroacetate, methyl 4,4,4-trifluorobutanoate, methyl difluoroacetate, 2,2,2-trifluoroethyl butanoate, methyl 2,2-difluoropropanoate, ethyl trifluoroacetate, 2,2-difluoroethyl trifluoroacetate, 2,2,2-trifluoroethyl acetate, ethyl 2,2-difluoroacetate, 2,2-difluoroethyl acetate, 2,2-difluoroethyl 3,3-difluoropropanoate, 2,2-difluoroethyl 2,2-difluoroacetate, succinonitrile, propionitrile, butyronitrile, adiponitrile, 1,3,6-hexanetricarbonitrile, trimethyl borate, triphenyl borate, triethyl borate, tris(pentafluorophenyl) borane, tris(trimethylsilyl)phosphate, tris(2,2,2-trifluoroethyl) borate, trimethyl phosphate, triethyl phosphate, tris(trimethylsilyl)phosphate, tris(trimethylsilyl)phosphite, tris(2,2,2-trifluoroethyl) phosphate, tris(2,2,2-trifluoroethyl) phosphite, (pentafluorophenyl) diphenyl phosphine, tris(pentafluorophenyl) phosphine, 1,3,2-dioxathiolane-2,2-dioxide, 1,3-propanesultone, prop-1-ene-1,3-sultone, propanediol cyclic sulfate, 1,3,2-dioxathiolane 2-oxide, bis(2,2,2-trifluoroethyl) ether, 1,1,2,2-tetrafluoroethyl-2,2,3,3-tetrafluoropropyl ether, 1H,1H,5H-octafluoropentyl-1,1,2,2-tetrafluoroethylether, 2,2,2-trifluoroethyl 1,1,2,2-tetrafluoroethyl ether, tetrahydrofuran, 1,3-dioxolane, 1,2-dimethoxyethane, diethylene glycol dimethyl ether, tetraethylene glycol dimethyl ether, succinic anhydride, butyric anhydride, 1,4-dioxane, tetravinyl silane, 1,4-butane sultone, dimethyl sulfoxide, methylene methanedisulfonate, N,N-dimethylformamide, gamma-butyrolactone, or a combination thereof; and a metal salt.

Aspect 24: An electrolyte comprising: difluoromethyl 3,3,3-trifluoropropanoate, 2,2-difluoroethyl 3,3,3-trifluoropropanoate, or a combination thereof; and a metal salt.

Aspect 25: The electrolyte of aspect 24, where the metal salt comprises lithium bis(trifluoromethanesulfonyl)imide, lithium bis(fluorosulfonyl)imide, lithium bis(oxalato)borate, lithium hexafluorophosphate, lithium tetrafluoroborate, lithium perchlorate, lithium hexafluoroarsenate, lithium triflate, lithium nitrate, lithium difluorophosphate, lithium difluoro(oxalato)borate, lithium trifluoroacetate, lithium 4,5-dicyano-2-(trifluoromethyl)imidazole, lithium difluoro(dioxalato)phosphate, lithium tetrafluoro(oxalato)phosphate, lithium bis(pentafluoroethanesulfonyl)imide, or a combination thereof.

Aspect 26: The electrolyte of aspect 24, where the metal salt comprises sodium bis(trifluoromethanesulfonyl)imide, sodium bis(fluorosulfonyl)imide, sodium bis(oxalato)borate, sodium hexafluorophosphate, sodium tetrafluoroborate, sodium perchlorate, sodium hexafluoroarsenate, sodium triflate, sodium nitrate, sodium difluorophosphate, sodium difluoro(oxalato)borate, sodium trifluoroacetate, sodium 4,5-dicyano-2-(trifluoromethyl)imidazole, or a combination thereof.

Aspect 27: The electrolyte of any one of aspects 24 to 26, further comprising fluoroethylene carbonate, propylene carbonate, dimethyl carbonate, diethyl carbonate, ethyl methyl carbonate, vinyl carbonate, vinyl ethylene carbonate, ethylene carbonate, difluoroethylene carbonate, 3,3,3-trifluoropropylene carbonate, monofluoroethyl methyl carbonate, difluoroethyl methyl carbonate, trifluoroethyl methyl carbonate, bis(2,2,2-trifluoroethyl)carbonate, 2,2-difluoroethyl ethyl carbonate, acetonitrile, fluoroacetonitrile, ethyl acetate, methyl acetate, methyl propanoate, methyl 3,3,3-trifluoropropanoate, ethyl 3,3,3-trifluoropropanoate, ethyl 2,2-difluoropropanoate, isopropyl trifluoroacetate, methyl 4,4,4-trifluorobutanoate, methyl difluoroacetate, 2,2,2-trifluoroethyl butanoate, methyl 2,2-difluoropropanoate, ethyl trifluoroacetate, 2,2-difluoroethyl trifluoroacetate, 2,2,2-trifluoroethyl acetate, ethyl 2,2-difluoroacetate, 2,2-difluoroethyl acetate, 2,2-difluoroethyl 3,3-difluoropropanoate, 2,2-difluoroethyl 2,2-difluoroacetate, succinonitrile, propionitrile, butyronitrile, adiponitrile, 1,3,6-hexanetricarbonitrile, trimethyl borate, triphenyl borate, triethyl borate, tris(pentafluorophenyl)borane, tris(trimethylsilyl)phosphate, tris(2,2,2-trifluoroethyl)borate, trimethyl phosphate, triethyl phosphate, tris(trimethylsilyl)phosphate, tris(trimethylsilyl)phosphite, tris(2,2,2-trifluoroethyl) phosphate, tris(2,2,2-trifluoroethyl) phosphite, (pentafluorophenyl)diphenyl phosphine, tris(pentafluorophenyl) phosphine, 1,3,2-dioxathiolane-2,2-dioxide, 1,3-propanesultone, prop-1-ene-1,3-sultone, propanediol cyclic sulfate, 1,3,2-dioxathiolane 2-oxide, bis(2,2,2-trifluoroethyl) ether, 1,1,2,2-tetrafluoroethyl-2,2,3,3-tetrafluoropropyl ether, 1H,1H,5H-octafluoropentyl-1,1,2,2-tetrafluoroethylether, 2,2,2-trifluoroethyl 1,1,2,2-tetrafluoroethyl ether, tetrahydrofuran, 1,3-dioxolane, 1,2-dimethoxyethane, diethylene glycol dimethyl ether, tetraethylene glycol dimethyl ether, succinic anhydride, butyric anhydride, 1,4-dioxane, tetravinyl silane, 1,4-butane sultone, dimethyl sulfoxide, methylene methanedisulfonate, N,N-dimethylformamide, gamma-butyrolactone, or a combination thereof.

Aspect 28: A battery comprising: the electrolyte of any one of aspect 24 to 27; an anode; a separator; and a cathode, wherein the anode comprises sodium metal, graphite, silicon, a silicon oxide, a graphite/silicon composite, a graphite/silicon oxide composite, a silicon/carbon composite, a silicon carbide, a graphite/silicon carbide composite, a silicon nitride, a graphite/silicon nitride composite, a hard carbon, an amorphous carbon, sodium titanium oxide, tin, a tin oxide, or a tin sulfide; and the cathode comprises sodium vanadium phosphate, sodium copper nickel iron, manganese oxide, Prussian white, Prussian blue, sodium iron manganese oxide, sodium nickel manganese oxide, sodium ferric phosphate pyrophosphate, sodium vanadium phosphate pyrophosphate, sodium ferric sulfate, sodium manganese oxide, sodium manganese phosphate, sodium nickel manganese iron oxide, sodium iron nickel oxide, sodium iron phosphate, sodium iron fluorophosphate, sodium iron manganese fluorophosphate, sodium vanadium fluorophosphate, sodium iron pyrophosphate, sodium manganese pyrophosphate, sodium vanadium pyrophosphate, a transition metal sulfide, sulfur, or a sulfur/carbon composite, or the anode comprises graphite, lithium metal, silicon, a silicon oxide, a graphite/silicon composite, a graphite/silicon oxide composite, a silicon/carbon composite, a silicon carbide, a graphite/silicon carbide composite, a silicon nitride, a graphite/silicon nitride composite, a hard carbon, an amorphous carbon, or lithium titanium oxide; and the cathode comprises lithium nickel manganese cobalt oxide, lithium nickel cobalt aluminum oxide, lithium nickel manganese aluminum oxide, lithium nickel manganese cobalt aluminum oxide, lithium nickel oxide, lithium nickel manganese oxide, lithium cobalt oxide, lithium manganese oxide, lithium and manganese-rich cathode, lithium iron phosphate, lithium cobalt phosphate, lithium manganese phosphate, lithium manganese iron phosphate, transition metal sulfide, sulfur, or a sulfur/carbon composite.

It should be understood that the foregoing description is only illustrative of the present disclosure. Various alternatives and modifications may be devised by those skilled in the art without departing from the disclosure. Accordingly, the present disclosure is intended to embrace all such alternatives, modifications and variances that fall within the scope of the appended claims.

Claims:

1. A fluorinated ester selected from difluoromethyl 3,3,3-trifluoropropanoate or 2,2-difluoroethyl 3,3,3-trifluoropropanoate.

2. A method of synthesizing a fluorinated ester, the method comprising:
(a) providing a carboxylic acid or carboxylic acid derivative; and
(b) reacting the carboxylic acid or carboxylic acid derivative with:
i. difluorocarbene; or
ii. a fluorinated alcohol;
to synthesize the fluorinated ester.

3. The method of claim 2, wherein the carboxylic acid or carboxylic acid derivative is fluorinated.

4. The method of claim 2, wherein the difluorocarbene is generated from (bromodifluoromethyl) trimethylsilane.

5. The method of claim 2, wherein the carboxylic acid comprises 3,3,3-trifluoropropanoic acid.

6. The method of claim 2, wherein the carboxylic acid derivative comprises a derivative of 3,3,3-trifluoropropanoic acid.

7. The method of claim 2, wherein the carboxylic acid derivative comprises an acid fluoride, an acid chloride, or an acid bromide.

8. The method of claim 2, wherein the fluorinated alcohol comprises 2,2-difluoroethanol.

9. A fluorinated ester synthesized by the method of claim 2, wherein the fluorinated ester is difluoromethyl 3,3,3-trifluoropropanoate.

10. A fluorinated ester synthesized by the method of claim 2, wherein the fluorinated ester is 2,2-difluoroethyl 3,3,3-trifluoropropanoate.

11. An electrolyte comprising:
difluoromethyl 3,3,3-trifluoropropanoate, 2,2-difluoroethyl 3,3,3-trifluoropropanoate, or a combination thereof; and
a metal salt.

12. The electrolyte of claim 11, wherein the metal salt comprises lithium bis(trifluoromethanesulfonyl)imide, lithium bis(fluorosulfonyl)imide, lithium bis(oxalato)borate, lithium hexafluorophosphate, lithium tetrafluoroborate, lithium perchlorate, lithium hexafluoroarsenate, lithium triflate, lithium nitrate, lithium difluorophosphate, lithium difluoro(oxalato)borate, lithium trifluoroacetate, lithium 4,5-dicyano-2-(trifluoromethyl)imidazole, lithium difluoro(dioxalato)phosphate, lithium tetrafluoro(oxalato)phosphate, lithium bis(pentafluoroethanesulfonyl)imide, or a combination thereof.

13. The electrolyte of claim 11, wherein the metal salt comprises sodium bis(trifluoromethanesulfonyl)imide, sodium bis(fluorosulfonyl)imide, sodium bis(oxalato)borate, sodium hexafluorophosphate, sodium tetrafluoroborate, sodium perchlorate, sodium hexafluoroarsenate, sodium triflate, sodium nitrate, sodium difluorophosphate, sodium difluoro(oxalato)borate, sodium trifluoroacetate, sodium 4,5-dicyano-2-(trifluoromethyl)imidazole, or a combination thereof.

14. The electrolyte of claim 11, further comprising fluoroethylene carbonate, propylene carbonate, dimethyl carbonate, diethyl carbonate, ethyl methyl carbonate, vinyl carbonate, vinyl ethylene carbonate, ethylene carbonate, difluoroethylene carbonate, 3,3,3-trifluoropropylene carbonate, monofluoroethyl methyl carbonate, difluoroethyl methyl carbonate, trifluoroethyl methyl carbonate, bis(2,2,2-trifluoroethyl) carbonate, 2,2-difluoroethyl ethyl carbonate, acetonitrile, fluoroacetonitrile, ethyl acetate, methyl acetate, methyl propanoate, methyl 3,3,3-trifluoropropanoate, ethyl 3,3,3-trifluoropropanoate, ethyl 2,2-difluoropropanoate, isopropyl trifluoroacetate, methyl 4,4,4-trifluorobutanoate, methyl difluoroacetate, 2,2,2-trifluoroethyl butanoate, methyl 2,2-difluoropropanoate, ethyl trifluoroacetate, 2,2-difluoroethyl trifluoroacetate, 2,2,2-trifluoroethyl acetate, ethyl 2,2-difluoroacetate, 2,2-difluoroethyl acetate, 2,2-difluoroethyl 3,3-difluoropropanoate, 2,2-difluoroethyl 2,2-difluoroacetate, succinonitrile, propionitrile, butyronitrile, adiponitrile, 1,3,6-hexanetricarbonitrile, trimethyl borate, triphenyl borate, triethyl borate, tris(pentafluorophenyl)borane, tris(trimethylsilyl)phosphate, tris(2,2,2-trifluoroethyl)borate, trimethyl phosphate, triethyl phosphate, tris(trimethylsilyl)phosphate, tris(trimethylsilyl)phosphite, tris(2,2,2-trifluoroethyl) phosphate, tris(2,2,2-trifluoroethyl)phosphite, (pentafluorophenyl)diphenyl phosphine, tris(pentafluorophenyl) phosphine, 1,3,2-dioxathiolane-2,2-dioxide, 1,3-propanesultone, prop-1-ene-1,3-sultone, propanediol cyclic sulfate, 1,3,2-dioxathiolane 2-oxide, bis(2,2,2-trifluoroethyl)ether, 1,1,2,2-tetrafluoroethyl-2,2,3,3-tetrafluoropropyl ether, 1H,1H,5H-octafluoropentyl-1,1,2,2-tetrafluoroethylether, 2,2,2-trifluoroethyl 1,1,2,2-tetrafluoroethyl ether, tetrahydrofuran, 1,3-dioxolane, 1,2-dimethoxyethane, diethylene glycol dimethyl ether, tetraethylene glycol dimethyl ether, succinic anhydride, butyric anhydride, 1,4-dioxane, tetravinyl silane, 1,4-butane sultone, dimethyl sulfoxide, methylene methanedisulfonate, N,N-dimethylformamide, gamma-butyrolactone, or a combination thereof.

15. A battery comprising:
the electrolyte of claim 11;
an anode;
a separator; and
a cathode,
wherein
the anode comprises sodium metal, graphite, silicon, a silicon oxide, a graphite/silicon composite, a graphite/silicon oxide composite, a silicon/carbon composite, a silicon carbide, a graphite/silicon carbide composite, a silicon nitride, a graphite/silicon nitride composite, a hard carbon, an amorphous carbon, sodium titanium oxide, tin, a tin oxide, or a tin sulfide; and the cathode comprises sodium vanadium phosphate, sodium copper nickel iron, manganese oxide, Prussian white, Prussian blue, sodium iron manganese oxide, sodium nickel manganese oxide, sodium ferric phosphate pyrophosphate, sodium vanadium phosphate pyrophosphate, sodium ferric sulfate, sodium manganese oxide, sodium manganese phosphate, sodium nickel manganese iron oxide, sodium iron nickel oxide, sodium iron phosphate, sodium iron fluorophosphate, sodium iron manganese fluorophosphate, sodium vanadium fluorophosphate, sodium iron pyrophosphate, sodium manganese pyrophosphate, sodium vanadium pyrophosphate, a transition metal sulfide, sulfur, or a sulfur/carbon composite, or
the anode comprises graphite, lithium metal, silicon, a silicon oxide, a graphite/silicon composite, a graphite/silicon oxide composite, a silicon/carbon composite, a silicon carbide, a graphite/silicon carbide composite, a silicon nitride, a graphite/silicon nitride composite, a hard carbon, an amorphous carbon, or lithium titanium oxide; and the cathode comprises lithium nickel manganese cobalt oxide, lithium nickel cobalt aluminum oxide, lithium nickel manganese aluminum oxide, lithium nickel manganese cobalt aluminum oxide, lithium nickel oxide, lithium nickel manganese oxide, lithium cobalt oxide, lithium manganese oxide, lithium and manganese-rich cathode, lithium iron phosphate, lithium cobalt phosphate, lithium manganese phosphate, lithium manganese iron phosphate, transition metal sulfide, sulfur, or a sulfur/carbon composite.
